SEQUENCE LISTING (41 sequences)

SEQ ID NO: 1 - protein, 28 aa, Artificial sequence
  synthetic sequence
GCPRILMRCKQDSDCLAGCVCGPNGFCG

SEQ ID NO: 2 - protein, 34 aa, Artificial sequence
  synthetic sequence
CVRLHESCLGQQVPCCDPAATCYCRFFNAFCYCR

SEQ ID NO: 3 - protein, 29 aa, Artificial sequence
  synthetic sequence
CGETCVGGTCNTPGCTCSWPVCTRNGLPV

SEQ ID NO: 4 - protein, 34 aa, Artificial sequence
  synthetic sequence
SGSDGGVCPKILKKCRRDSDCPGACICRGNGYCG

SEQ ID NO: 5 - protein, 36 aa, Artificial sequence
  synthetic sequence
MCMPCFTTDHQMARKCDDCCGGKGRGKCYGPQCLCR

SEQ ID NO: 6 - protein, 33 aa, Artificial sequence
  synthetic sequence
GCPQGRGDWAPTSCKQDSDCRAGCVCGPNGFCG

SEQ ID NO: 7 - protein, 33 aa, Artificial sequence
  synthetic sequence
GCPRPRGDNPPLTCSQDSDCLAGCVCGPNGFCG

SEQ ID NO: 8 - protein, 33 aa, Artificial sequence
  synthetic sequence
GCAEPRGDMPWTWCKQDSDCLAGCVCGPNGFCG

SEQ ID NO: 9 - protein, 33 aa, Artificial sequence
  synthetic sequence
GCVGGRGDWSPKWCKQDSDCPAGCVCGPNGFCG

SEQ ID NO: 10 - protein, 33 aa, Artificial sequence
  synthetic sequence
GCAELRGDRSYPECKQDSDCLAGCVCGPNGFCG

SEQ ID NO: 11 - protein, 33 aa, Artificial sequence
  synthetic sequence
GCRLPRGDVPRPHCKQDSDCQAGCVCGPNGFCG

SEQ ID NO: 12 - protein, 33 aa, Artificial sequence
  synthetic sequence
GCYPLRGDNPYAACKQDSDCRAGCVCGPNGFCG

SEQ ID NO: 13 - protein, 33 aa, Artificial sequence
  synthetic sequence
GCTIGRGDWAPSECKQDSDCLAGCVCGPNGFCG

SEQ ID NO: 14 - protein, 33 aa, Artificial sequence
  synthetic sequence
GCHPPRGDNPPVTCKQDSDCLAGCVCGPNGFCG

SEQ ID NO: 15 - protein, 33 aa, Artificial sequence
  synthetic sequence
GCPEPRGDNPPPSCKQDSDCRAGCVCGPNGFCG

SEQ ID NO: 16 - protein, 33 aa, Artificial sequence
  synthetic sequence
GCLPPRGDNPPPSCKQDSDCQAGCVCGPNGFCG

SEQ ID NO: 17 - protein, 33 aa, Artificial sequence
  synthetic sequence
GCHLGRGDWAPVGCKQDSDCPAGCVCGPNGFCG

SEQ ID NO: 18 - protein, 33 aa, Artificial sequence
  synthetic sequence
GCNVGRGDWAPSECKQDSDCPAGCVCGPNGFCG

SEQ ID NO: 19 - protein, 33 aa, Artificial sequence
  synthetic sequence
GCFPGRGDWAPSSCKQDSDCRAGCVCGPNGFCG

SEQ ID NO: 20 - protein, 33 aa, Artificial sequence
  synthetic sequence
GCPLPRGDNPPTECKQDSDCQAGCVCGPNGFCG

SEQ ID NO: 21 - protein, 33 aa, Artificial sequence
  synthetic sequence
GCSEARGDNPRLSCKQDSDCRAGCVCGPNGFCG

SEQ ID NO: 22 - protein, 33 aa, Artificial sequence
  synthetic sequence
GCLLGRGDWAPEACKQDSDCRAGCVCGPNGFCG

SEQ ID NO: 23 - protein, 33 aa, Artificial sequence
  synthetic sequence
GCHVGRGDWAPLKCKQDSDCQAGCVCGPNGFCG

SEQ ID NO: 24 - protein, 33 aa, Artificial sequence
  synthetic sequence
GCVRGRGDWAPPSCKQDSDCPAGCVCGPNGFCG

SEQ ID NO: 25 - protein, 33 aa, Artificial sequence
  synthetic sequence
GCLGGRGDWAPPACKQDSDCRAGCVCGPNGFCG

SEQ ID NO: 26 - protein, 33 aa, Artificial sequence
  synthetic sequence
GCFVGRGDWAPLTCKQDSDCQAGCVCGPNGFCG

SEQ ID NO: 27 - protein, 33 aa, Artificial sequence
  synthetic sequence
GCPVGRGDWSPASCKQDSDCRAGCVCGPNGFCG

SEQ ID NO: 28 - protein, 33 aa, Artificial sequence
  synthetic sequence
GCPRPRGDNPPLTCKQDSDCLAGCVCGPNGFCG

SEQ ID NO: 29 - protein, 33 aa, Artificial sequence
  synthetic sequence
GCYQGRGDWSPSSCKQDSDCPAGCVCGPNGFCG

SEQ ID NO: 30 - protein, 33 aa, Artificial sequence
  synthetic sequence
GCAPGRGDWAPSECKQDSDCQAGCVCGPNGFCG

SEQ ID NO: 31 - protein, 33 aa, Artificial sequence
  synthetic sequence
GCVQGRGDWSPPSCKQDSDCPAGCVCGPNGFCG

SEQ ID NO: 32 - protein, 33 aa, Artificial sequence
  synthetic sequence
GCHVGRGDWAPEECKQDSDCQAGCVCGPNGFCG

SEQ ID NO: 33 - protein, 33 aa, Artificial sequence
  synthetic sequence
GCDGGRGDWAPPACKQDSDCRAGCVCGPNGFCG

SEQ ID NO: 34 - protein, 33 aa, Artificial sequence
  synthetic sequence
GCPQGRGDWAPEWCKQDSDCPAGCVCGPNGFCG

SEQ ID NO: 35 - protein, 33 aa, Artificial sequence
  synthetic sequence
GCPRGRGDWSPPACKQDSDCQAGCVCGPNGFCG

SEQ ID NO: 36 - protein, 38 aa, Artificial sequence
  synthetic sequence
GCVRLHESCLGQQVPCCDPAATCYCSGRGDNDLVCYCR

SEQ ID NO: 37 - protein, 38 aa, Artificial sequence
  synthetic sequence
GCVRLHESCLGQQVPCCDPAATCYCKGRGDARLQCYCR

SEQ ID NO: 38 - protein, 38 aa, Artificial sequence
  synthetic sequence
GCVRLHESCLGQQVPCCDPAATCYCVGRGDDNLKCYCR

SEQ ID NO: 39 - protein, 38 aa, Artificial sequence
  synthetic sequence
GCVRLHESCLGQQVPCCDPAATCYCEGRGDRDMKCYCR

SEQ ID NO: 40 - protein, 38 aa, Artificial sequence
  synthetic sequence
GCVRLHESCLGQQVPCCDPAATCYCYGRGDNDLRCYCR

SEQ ID NO: 41 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2022/0211861 A1
(43) Pub. Date: Jul. 7, 2022

(54) DRUG CONJUGATES AND METHODS OF USING SAME

(71) Applicant: The Board of Trustees of the Leland Staford Junior University, Stanford, CA (US)

(72) Inventors: Jennifer R. Cochran, Stanford, CA (US); James R. Kintzing, Stanford, CA (US); Caitlyn Miller, Stanford, CA (US)

(21) Appl. No.: 17/609,284

(22) PCT Filed: May 21, 2020
(86) PCT No.: PCT/US2020/034050

§ 371 Date: Nov. 5, 2021

Related U.S. Application Data
(60) Provisional application No. 62/851,443, filed on May 22, 2019.

(51) Int. Cl. A61K 47/64; A61K 47/65; A61K 47/68; A61K 47/55; A61K 47/54; A61P 35/00

(52) U.S. Cl. CPC A61K 47/6415; A61K 47/64; A61K 47/65; A61K 47/6817; A61P 35/00; A61K 47/549; A61K 47/6865; A61K 47/6851; A61K 47/55

(57) ABSTRACT

Provided are conjugates. In certain aspects, provided are drug conjugates that include a knottin peptide comprising an engineered loop that binds to a cell surface molecule, and an anti-microtubule agent conjugated to the knottin peptide via a linker. In some aspects, provided are drug conjugates that include a fusion protein that includes a knottin peptide comprising an engineered loop that binds to a cell surface molecule, fused to an antibody subunit or fragment thereof. Such drug conjugates further include a drug conjugated to the fusion protein. Also provided are compositions and kits that include the conjugates of the present disclosure. Methods of using the conjugates, e.g., for therapeutic purposes, are also provided.

### CROSS-REFERENCE TO RELATED APPLICATIONS

This application claims the benefit of U.S. Provisional Patent Application No. 62/851,443, filed May 22, 2019, which application is incorporated herein by reference in its entirety.

### INTRODUCTION

Cancer chemotherapeutics are often limited by a narrow therapeutic window—the difference between the minimum effective dose (that kills the tumor) and the maximum tolerated dose (that causes significant side effects). Targeted drug delivery provides a way to increase local concentration at the tumor while decreasing off-target toxicity. Antibody-drug conjugates (ADCs) constitute the primary current platform for targeted drug delivery. ADCs have shown substantial clinical success against blood tumors but have been limited in their efficacy against many solid tumors and other difficult tumor targets, e.g., brain tumors, pancreatic tumors, etc. Due to their relatively high molecular weight (MW), ADCs are hindered in their ability to homogenously penetrate solid tumors. There is a need for alternative targeting proteins with altered pharmacokinetics to improve the efficacy of targeted therapies against solid tumors and other difficult tumor targets.

### SUMMARY

Provided are conjugates. In certain aspects, provided are drug conjugates that include a knottin peptide comprising an engineered loop that binds to a cell surface molecule, and an anti-microtubule agent conjugated to the knottin peptide via a linker. In some aspects, provided are drug conjugates that include a fusion protein that includes a knottin peptide comprising an engineered loop that binds to a cell surface molecule, fused to an antibody subunit or fragment thereof. Such drug conjugates further include a drug conjugated to the fusion protein. Also provided are compositions and kits that include the conjugates of the present disclosure. Methods of using the conjugates, e.g., for therapeutic purposes, are also provided.

### BRIEF DESCRIPTION OF THE FIGURES

FIG. 1 (a) Schematic illustration of Val-Cit-PAB-MMAE, a derivative of MMAE which is stable extracellularly and cleaved upon cellular internalization via cathepsin proteases. (b) Schematic illustrations of Knottin, knottin-Fc, and knottin-Ab constructs conjugated to the MMAE derivative according to some embodiments of the present disclosure.

FIG. 2 Amino acid sequences for EETI 2.5F, 2.5Z, and 3CM. Sequence of EETI-2.5F, EETI-2.5Z, and EETI 3CM with integrin-binding loop and substitution sites highlighted in red and disulfide linkages of the cystine-knot scaffold depicted in yellow. Position 15 (red X₁) indicates the site where an azide-containing unnatural amino acid, 5-azido-I-norvaline, was substituted to allow for site-specific bioconjugation of KDC. Position 31 (red X₂) indicates the site where phenylalanine was replaced with tyrosine to allow for improved concentration measurement using UV-Vis spectroscopy.

FIG. 3 Preparation of 2.5F-Val-Cit-PAB-MMAE KDC. (a) Val-Cit-PAB-MMAE is a derivative of MMAE known to be stable extracellularly and cleaved upon cellular internalization via cathepsin proteases. (b) The knottin-drug conjugate (KDC) was prepared from azido-knottin, prepared via solid phase peptide synthesis and containing 5-azidonorvaline, conjugated to Val-Cit-PAB-MMAE using copper-free click chemistry.

FIG. 4 LC-MS and UV chromatograms for K and KDC. (a) UV chromatogram and (b) LC-MS for 3CM knottin peptide (K). Method consists of a linear gradient from 10% to 46% solvent B over 16 minutes. (c) UV chromatogram and (d) LC-MS for 3CM-Val-Cit-PAB-MMAE knottin-drug conjugate (KDC). Method consists of a 2-minute isocratic hold at 30% solvent B, followed by a linear gradient from 30% to 100% solvent B over 15 minutes.

FIG. 5 Preparation of 2.5F-Ab-Val-Cit-PAB-MMAE KFDC. (a) Val-Cit-PAB-MMAE is a derivative of MMAE and is known to be stable extracellularly and cleaved upon cellular internalization via cathepsin proteases. (b) The knottin-Fc-drug conjugate (KFDC) was prepared by Hydrazino-Pictet-Spengler ligation1 of aldehyde tagged KFc with a HIPS linker MMAE derivative.

FIG. 6 Mass spectrometry data confirming production of KFDC.

FIG. 7 Preparation of 2.5F-Ab-Val-Cit-PAB-MMAE KADC. (a) Val-Cit-PAB-MMAE is a derivative of MMAE and is known to be stable extracellularly and cleaved upon cellular internalization via cathepsin proteases. (b) The knottin-Ab-drug conjugate (KADC) was prepared by Hydrazino-Pictet-Spengler ligation1 of aldehyde tagged (α-FITC) KAb with a HIPS linker MMAE derivative.

FIG. 8 Mass spectrometry data confirming production of KADC.

FIG. 9 Binding affinity of knottin-targeted proteins on U87MG cells. (a) Direct binding of AF488-labeled K, KFc, and KAb to U87MG cells (Kd=0.35 nM for K, 2.5 nM for KFc, and 2.3 nM for KAb). (b) Indirect binding of drug conjugates competing off 1 nM AF488-2.5F (Ki=0.9 for KDC, 1.2 for KFc, and 1.3 for KAb).

FIG. 10 Internalization of knottin-based proteins. (a) Cells incubated with only the anti-Alexa Fluor 488 quenching antibody or incubated at 4° C. (to freeze the cell membrane and inhibit internalization) had detectable fluorescence above cells only. (b) Internalization of AF488-labled K, KFc, and KAb. (c) Internalization of bivalent KFc compared to monovalent, one-armed KFc. (d)C-terminal knottin-antibody fusion (AbK) internalization compared to N-terminal KAb and KFc.

FIG. 11 Effect of denatured media and PBSA on internalization. Serum proteins, including c1q, are capable of interacting with antibody Fc regions and may influence cellular internalization. To test if serum proteins were influencing internalization rate, cells were incubated with AF488-labeled protein in complete media (DMEM), heat-denatured DMEM, or in phosphate buffered saline with 0.1% BSA (PBSA).

FIG. 12 Effect of Fc-blocker and excess K on internalization. (a) Internalization of AF488-K, AF488-K co-administered with excess unlabeled K (K′), and non-binding CTRL knottin. (b) Internalization of AF488-KFc, AF488-KFc co-administered with Fc blocker, AF488-KFc co-administered with excess K′, and non-binding CTRL-Fc. (c) Internalization of one-armed, mono-AF488-KFc, mono-AF488-KFc co-administered with Fc blocker, and mono-AF488-KFc co-administered with excess K′.

FIG. 13 PDC inhibition of in-vitro cell proliferation. U87MG cells treated with MMAE compared to (a), KDC and unconjugated K, (b) KFDC and unconjugated KFc, and (c) KADC and unconjugated KAb. Cell proliferation at 120 hours was assessed by measuring the absorbance resulting from CCK8 reagent at λ=450 nm and is reported as the percent maximum relative to untreated cells. All drug conjugates were tested again in an additional experiment and plotted together in (d).

FIG. 14 PDC Inhibition of proliferation and cytotoxicity over time. RFP-expressing U87MG cells were treated with MMAE or drug conjugates and proliferation and cytotoxicity (via SYTOX green) were measured over time via IncuCyte imaging. (a) Comparison of proliferation between cells washed with fresh media containing the same treatment conditions after 24 h. (b) Comparison of proliferation between cells washed with fresh, drug-free media after 24 h. (c) Comparison of cytotoxicity between cells washed with fresh media containing the same treatment conditions after 24 h. (d) Comparison of cytotoxicity between cells washed with fresh, drug-free media after 24 h.

FIG. 15 PDC inhibition proliferation after 3h incubation. RFP-expressing U87MG cells were treated with MMAE or drug conjugates and proliferation was measured over time via IncuCyte imaging. Comparison of proliferation between cells washed with fresh, drug-free media after 3 h.

FIG. 16 In vivo imaging of AF680-labeled PDCS. Mice bearing U87MG hip xenografts were injected with 1.5 nmol of (a) AF680-KDC, (b) AF680-KFDC, or (c) AF680-KADC. Mice (n=3) were imaged periodically using 640 nm excitation and 710 nm emission filters. Single mice from each treatment group are shown at selected time points to illustrate differences in clearance and localization rates.

FIG. 17 Pharmacokinetics of PDCs. Quantification of radiant efficiency in the tumor or an equivalent area centered over the shoulder (background) of mice treated with (a) AF680-KDC, (b) AF680-KFDC, or (c) AF680-KADC. Circulatory half-lives were estimated by fitting a biphasic exponential decay curve to the background data. (d-f) Tumor-to-background ratios were calculated as the radiant efficiency in the tumor divided by the radiant efficiency of an equivalent are centered over the shoulder.

FIG. 18 Biodistribution of PDCs. Mice bearing U87MG xenograft tumors were injected with 1.5 nmol of fluorescently labeled drug conjugates. Tumor, liver, kidneys, spleen, pancreas, heart, lungs, bladder, quadriceps (muscle), and sternum (bone) were removed 4 and 24 h post-administration for imaging.

FIG. 19 Tumor growth of Nu/Nu mice bearing U87MG hip xenografts treated three times per week for three weeks with PBS, KDC, KFDC, or KADC. Each dose contained the appropriate drug conjugate normalized to 2.38 nmol MMAE (˜0.6 mg/kg KDC, ˜5 mg/kg KFDC, ˜10 mg/kg KADC). Endpoint criterion was tumor area >100 mm². (a) Kaplan-Meier survival curve. (b) Average tumor area for each treatment group. Mice that reached euthanasia criteria were included in the average as their last recorded tumor area. (c) Tumor measurements for individual mice within each group. (d) Average weight normalized to starting weight.

FIG. 20 Tumor growth of Nu/Nu mice bearing U87MG hip xenografts treated once weekly for four weeks with PBS, unconjugated K, or 1 mg/kg or 5 mg/kg KDC. Endpoint criterion was tumor area >150 mm². (a) Kaplan-Meier survival curve. (b) Average tumor area for each treatment group. Mice that reached euthanasia criteria were included in the average as their last recorded tumor area. (c) Tumor measurements for individual mice within each group.

FIG. 21 Tumor growth of Nu/Nu mice bearing U87MG hip xenografts treated once weekly for four weeks with PBS, unconjugated KFc, or 5 mg/kg or 10 mg/kg KFDC. Endpoint criterion was tumor area >150 mm². (a) Kaplan-Meier survival curve. (b) Average tumor area for each treatment group. Mice that reached euthanasia criteria were included in the average as their last recorded tumor area. (c) Tumor measurements for individual mice within each group.

FIG. 22 Tumor growth of Nu/Nu mice bearing U87MG hip xenografts treated once weekly for four weeks with PBS, unconjugated (α-CEA) KAb, or 5 mg/kg or 10 mg/kg KADC. Endpoint criterion was tumor area >150 mm². (a) Kaplan-Meier survival curve. (b) Average tumor area for each treatment group. Mice that reached euthanasia criteria were included in the average as their last recorded tumor area. (c) Tumor measurements for individual mice within each group.

FIG. 23 (a) Kaplan-Meier survival curve. (b) Average tumor area for each treatment group. Mice that reached euthanasia criteria were included in the average as their last recorded tumor area. (c) Tumor measurements for individual mice within each group.

FIG. 24 Therapeutic efficacy of KADC dosed at 30 mg/kg. Tumor growth of Nu/Nu mice bearing U87MG hip xenografts treated once weekly for four weeks with PBS, 10 mg/kg KADC, or 30 mg/kg KADC. Endpoint criterion was tumor area >150 mm². (a) Kaplan-Meier survival curve. (b) Average tumor area for each treatment group. Mice that reached euthanasia criteria were included in the average as their last recorded tumor area. (c) Tumor measurements for individual mice within each group.

FIG. 25 Therapeutic efficacy of co-administration of KADC and KAb. Tumor growth of Nu/Nu mice bearing U87MG hip xenografts treated once weekly for four weeks with PBS, 10 mg/kg KADC, or 10 mg/kg KADC co-administered with 20 mg/kg KAb. Endpoint criterion was tumor area >150 mm². (a) Kaplan-Meier survival curve. (b) Average tumor area for each treatment group. Mice that reached euthanasia criteria were included in the average as their last recorded tumor area. (c) Tumor measurements for individual mice within each group.

FIG. 26 Liver and kidney histology for KDC, KFDC, and KADC. Representative slices of livers and kidneys fixed with 4% PFA and stained with hematoxylin and eosin to visualize cellular structures. Slides were scored by an independent vet pathologist and all liver and kidney samples were confirmed to have no evidence of acute toxicity.

FIG. 27 Diffusion into solid tumors. Schematic illustration representing Krough cylinder diffusion of drug conjugates away from vasculature and into a solid tumor. Krough cylinder radius is limited by diffusivity and rate of cell binding and internalization.

FIG. 28 Confocal imaging of tumor spheroids. Confocal imaging showing diffusion of AF488-labeled drug conjugates into cell RFP-expressing U87MG spheroids. Red channel shows signal attenuation due to optical density; green channel shows intensity of fluorescently labeled proteins. All images are taken at a depth of ˜100 μm from the spheroid surface.

FIG. 29 Quantification of tumor spheroid imaging. Spheroid images are qualitatively useful, but quantification is challenging due to the non-uniform optical density. Red channel shows signal attenuation due to optical density while green channel shows fluorescently labeled protein. For each channel, radial intensity was calculated using FIJI image analysis software and plotted as a function of distance. (a) Average intensity as a function of distance for n=5 spheroids treated with AF488-KDC. (b) Average intensity as a function of distance for n=5 spheroids treated with AF488-KFDC. (c) Average intensity as a function of distance for n=5 spheroids treated with AF488-KADC. (d) Average intensity as a function of distance for n=5 spheroids treated with AF488-KADC and unlabeled K. (e) Relative green intensity corrected using relative red intensity. (f) Total integrated intensity for confocal images taken at 100 μm.

FIG. 30 Z-stack confocal imaging of tumor spheroids. Confocal imaging showing diffusion of AF488-labeled drug conjugates into cell RFP-expressing U87MG spheroids at 10 μm intervals from 50-90 μm from the spheroid surface.

FIG. 31 Killing of tumor spheroids in vitro. U87MG tumor spheroids were grown for 3 d before treatment with varying concentrations of KDC, KFDC, KADC, or MMAE. After 4 d incubation, SYTOX green was added and spheroids were agitated to measure cytotoxicity. Percent spheroid death was estimated by comparing green signal to spheroids killed with lysis buffer.

FIG. 32 IncuCyte imaging of tumor spheroids in vitro. RFP-expressing U87MG tumor spheroids were grown for 3 d before treatment with 100 uM of KDC, KADC, or MMAE. Cell death is visualized with SYTOX green. Images are taken 120 h after treatment.

### DETAILED DESCRIPTION

Before the conjugates, compositions and methods of the present disclosure are described in greater detail, it is to be understood that the conjugates, compositions and methods are not limited to particular embodiments described, as such may, of course, vary. It is also to be understood that the terminology used herein is for the purpose of describing particular embodiments only, and is not intended to be limiting, since the scope of the conjugates, compositions and methods will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the conjugates, compositions and methods. The upper and lower limits of these smaller ranges may independently be included in the smaller ranges and are also encompassed within the conjugates, compositions and methods, subject to any specifically excluded limit in the stated range. Where the stated range includes one or both of the limits, ranges excluding either or both of those included limits are also included in the conjugates, compositions and methods.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the conjugates, compositions and methods belong. Although any conjugates, compositions and methods similar or equivalent to those described herein can also be used in the practice or testing of the conjugates, compositions and methods, representative illustrative conjugates, compositions and methods are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present conjugates, compositions and methods are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the conjugates, compositions and methods, which are, for clarity, described in the context of separate embodiments, may also be provided in combination in a single embodiment. Conversely, various features of the conjugates, compositions and methods, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present conjugates, compositions and methods and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### Conjugates

As summarized above, the present disclosure provides conjugates. For example, described herein is the development and characterization of a toolbox of peptide-based drug conjugates as alternative targeting agents for cancer therapy. Traditional cancer chemotherapy agents often have narrow ranges between effective and toxic doses. To improve efficacy and minimize side effects, the protein-drug conjugates (PDCs) find use in selectively delivering chemotherapeutics to cancerous tissue.

The conjugates of the present disclosure include a knottin peptide that includes an engineered loop that binds to a cell surface molecule. The type of knottin peptide employed in the conjugates of the present disclosure may vary. Non-limiting examples of a knottin peptide that may be employed include an EETI-II peptide, an AgRP peptide, a w-conotoxin peptide, a Kalata B1 peptide, an MCoTI-II peptide, an agatoxin peptide, and a chlorotoxin peptide. The three-dimensional structure of a knottin peptide is minimally defined by a particular arrangement of three disulfide bonds. This characteristic topology forms a molecular knot in which one disulfide bond passes through a macrocycle formed by the other two intra-chain disulfide bridges. Although their secondary structure content is generally low, knottins share a small triple-stranded antiparallel β-sheet, which is stabilized by the disulfide bond framework. Folding and functional activity of knottins are often mediated by loop regions that are diverse in both length and amino acid composition. While three disulfide bonds are the minimum number that defines the fold of this family of peptides, knottins can also contain additional cysteine residues, yielding molecules with four or more disulfide bonds and additional constrained loops in their structure. The term “cystine” refers to a Cys residue in which the sulfur group is linked to another amino acid though a disulfide linkage; the term “cysteine” refers to the —SH (“half cystine”) form of the residue. Binding loop portions may be adjacent to cystines, such that there are no other intervening cystines in the primary sequence in the binding loop.

The knottin peptide may be a peptide described in the online KNOTTIN database, which includes detailed amino acid sequence, structure, classification and function information for thousands of polypeptides identified as contain cystine-knot motifs. Knottins are found in a variety of plants, animals, insects and fungi.

The knottin peptide may be full-length (that is, the length of the wild-type peptide/polypeptide), the knottin peptide may be truncated relative to the length of the wild-type peptide/polypeptide, or the knottin peptide may include additional amino acids such that the peptide is greater in length relative to the length of the wild-type peptide/polypeptide.

According to certain embodiments, a knottin-drug conjugate (KDC) of the present disclosure includes a knottin peptide based on any one of an Ecballium elaterium trypsin inhibitor II (EETI-II) peptide, an agouti-related protein (AgRP) peptide, a ω-conotoxin peptide, a Kalata B1 peptide, an MCoTI-II peptide, an agatoxin peptide, or a chlorotoxin peptide. In some embodiments, the knottin peptide is based on an Ecballium elaterium trypsin inhibitor II (EETI-II) peptide. In some embodiments, the knottin peptide is based on an agouti-related protein (AgRP) peptide.

By “EETI” is meant Protein Data Bank Entry (PDB) 2ETI. Its entry in the KNOTTIN database is EETI-II. In certain aspects, a knottin peptide of a conjugate of the present disclosure is based on an EETI-II peptide having the following amino acid sequence:

(SEQ ID NO: 1)
GCPRILMRCKQDSDCLAGCVCGPNGFCG

By “AGRP” is meant PDB entry 1 HYK and KNOTTIN database entry SwissProt AGRP_HUMAN. AGRP is a 132 amino acid neuropeptide that binds to melanocortin receptors in the human brain and is involved in regulating metabolism and appetite. The biological activity of AgRP is mediated by its C-terminal cysteine knot domain, which contains five disulfide bonds, but a fully active 34 amino acid truncated AgRP that contains only four disulfide bonds has been developed. In certain aspects, a knottin peptide of a conjugate of the present disclosure is based on a truncated AGRP peptide having the following amino acid sequence:

(SEQ ID NO: 2)
CVRLHESCLGQQVPCCDPAATCYCRFFNAFCYCR

According to certain embodiments, a knottin peptide of a conjugate of the present disclosure is based on a Kalata B1 peptide having the following amino acid sequence:

(SEQ ID NO: 3)
CGETCVGGTCNTPGCTCSWPVCTRNGLPV

In certain aspects, a knottin peptide of a conjugate of the present disclosure is based on a MCoTI-II peptide having the following amino acid sequence:

(SEQ ID NO: 4)
SGSDGGVCPKILKKCRRDSDCPGACICRGNGYCG

According to certain embodiments, a knottin peptide of a conjugate of the present disclosure is based on a chlorotoxin peptide having the following amino acid sequence:

(SEQ ID NO: 5)
MCMPCFTTDHQMARKCDDCCGGKGRGKCYGPQCLCR

Sequences and structural (e.g., loop) information for EETI-II, AgRP, ω-conotoxin, Kalata B1, MCoTI-II, agatoxin, chlorotoxin, and other knottin peptides upon which the knottin peptides of the conjugates of the present disclosure may be based may be found in the PDB, the KNOTTIN database, and other protein databases.

The knottin peptide includes an engineered loop that binds to a cell surface molecule—that is, the loop is engineered to bind to a target molecule on the surface of a cell. Knottins contain three disulfide bonds interwoven into a molecular ‘knot’ that constrain loop regions to a core of anti-parallel β-sheets. Wild-type EETI, for example, is composed of 28 amino acids with three disulfide-constrained loops: loop 1 (the trypsin binding loop, residues 3-8), loop 2 (residues 10-14), and loop 3 (residues 22-26) Knottin family members, which include protease inhibitors, toxins, and antimicrobials, share little sequence homology apart from their core cysteine residues. As a result, their disulfide-constrained loops tolerate much sequence diversity, making knottins amenable for protein engineering applications where mutations need to be introduced into a protein without abolishing its three-dimensional fold.

The engineered loop may include amino acid substitutions, insertions, and/or deletions in an existing loop of the knottin peptide, or the engineered loop may be a loop added to the knottin protein. That is, the knottin peptide of the conjugate may include a loop in addition to the one or more loops present in the wild-type peptide. By combining directed evolution with computational covariance analysis, guidelines for introducing modifications (both in amino acid sequence and loop length) into the loop regions of the knottin scaffold have been elucidated. See, e.g., Lahti et al. (2009) PLoS Comput. Biol. 5(9): e1000499.

In some embodiments, the loop of the knottin is engineered to bind to a cancer cell surface molecule. By “cancer cell” is meant a cell exhibiting a neoplastic cellular phenotype, which may be characterized by one or more of, for example, abnormal cell growth, abnormal cellular proliferation, loss of density dependent growth inhibition, anchorage-independent growth potential, ability to promote tumor growth and/or development in an immunocompromised non-human animal model, and/or any appropriate indicator of cellular transformation. “Cancer cell” may be used interchangeably herein with “tumor cell”, “malignant cell” or “cancerous cell”, and encompasses cancer cells of a solid tumor, a semi-solid tumor, a primary tumor, a metastatic tumor, and the like. Such an engineered loop confers upon the knottin peptide a cancer cell surface molecular recognition property that is not present in the wild-type peptide. In certain aspects, the cancer is a cancer known to have one or more tumor-associated or tumor-specific cell surface molecules (e.g., cell surface receptors, membrane proteases, and the like) and the engineered loop of the knottin peptide is engineered to bind to an extracellular domain of one or more such tumor-associated or tumor-specific cell surface molecules. By “tumor-associated cell surface molecule” is meant a cell surface molecule expressed on malignant cells with limited expression on cells of normal tissues, or a cell surface molecule expressed at much higher density on malignant versus normal cells.

Any tumor-associated cell surface molecule or tumor-specific cell surface molecule may be targeted by the knottin peptide of a conjugate of the present disclosure. In certain aspects, the target on the cancer cell surface to which the loop is engineered to bind is HER2, B7-H3 (CD276), CD19, CD20, GD2, CD22, CD30, CD33, CD56, CD66/CEACAM5, CD70, CD74, CD79b, CD123, CD133 CD138, CD171, B-cell maturation antigen (BCMA), Nectin-4, Mesothelin, Transmembrane glycoprotein NMB (GPNMB), Prostate-Specific Membrane Antigen (PSMA), SLC44A4, CA6, tyrosine-protein kinase Met (c-Met), epidermal growth factor receptor variant III (EGFRvIII), mucin 1 (MUC1), ephrin type-A receptor 2 (EphA2), glypican 2 (GPC2), glypican 3 (GPC3), fms-like tyrosine kinase 3 (FLT3), folate receptor alpha (FRα), IL-13 receptor alpha 2 (IL13Rα2), fibroblast activation protein (FAP), receptor tyrosine kinase-like orphan receptor 1 (ROR1), delta-like 3 (DLL3), κ light chain, vascular endothelial growth factor receptor 2 (VEGFR2), Trophoblast glycoprotein (TPBG), anaplastic lymphoma kinase (ALK), CA-IX, an integrin, C—X—C chemokine receptor type 4 (CXCR4), neuropilin-1 (NRP1), matriptase, and any other tumor-associated or tumor-specific molecules of interest.

According to certain embodiments, the target on the cancer cell surface is a receptor, e.g., a cell adhesion receptor, a receptor for a soluble factor (e.g., a growth factor, chemokine, or other soluble factor receptor), an immune cell receptor, or the like. In certain aspects, when the receptor is a cell adhesion receptor, the receptor is an integrin. For example, a conjugate of the present disclosure may include a knottin peptide having a loop engineered to bind to any one of αvβ1 integrin, αvβ3 integrin, αvβ5 integrin, αvβ6 integrin, α5β1 integrin, or any combination thereof. According to certain embodiments, the engineered loop binds to each of αvβ1 integrin, αvβ3 integrin, αvβ5 integrin, αvβ6 integrin, and α5β1 integrin.

An EETI-based knottin peptide (designated EETI-2.5D) having an engineered binding loop that binds to each of αvβ1 integrin, αvβ3 integrin, αvβ5 integrin, αvβ6 integrin, and α5β1 integrin, which may be employed in a conjugate of the present disclosure, has the following amino acid sequence (with the integrin-binding loop underlined):

(SEQ ID NO: 6)
GCPQGRGDWAPTSCKQDSDCRAGCVCGPNGFCG

An EETI-based knottin peptide (designated EETI-2.5F) having an engineered binding loop that binds to each of αvβ1 integrin, αvβ3 integrin, αvβ5 integrin, αvβ6 integrin, and α5β1 integrin, which may be employed in a conjugate of the present disclosure, has the following amino acid sequence (with the integrin-binding loop underlined):

(SEQ ID NO: 7)
GCPRPRGDNPPLTCSQDSDCLAGCVCGPNGFCG

In some embodiments, the knottin peptide of a conjugate of the present disclosure is an integrin-binding EETI-based knottin peptide as set forth in Table 1.

TABLE 1
Example EETI Intedrin-Binding Knottin Peptides

| Peptide | | |
|---|---|---|
| identifier | Sequence | SEQ ID NO: |
| 1.4A | GCAEP [symbol] MPWTWCKQDSDCLAGCVCGPNGFCG | (SEQ ID NO: 8) |
| 1.4B | GCVGGRGDWSPKWCKQDSDCPAGCVCGPNGFCG | (SEQ ID NO: 9) |
| 1.40 | GCAELRGDRSYPECKQDSDCLAGCVCGPNGFCG | (SEQ ID NO: 10) |
| 1.4E | GCRLPRGDVPRPHCKQDSDCQAGCVCGPNGFCG | (SEQ ID NO: 11) |
| 1.4H | GCYPLRGDNPYAACKQDSDCRAGCVCGPNGFCG | (SEQ ID NO: 12) |
| 1.5B | GCTIGRGDWAPSECKQDSDCLAGCVCGPNGFCG | (SEQ ID NO: 13) |
| 1.5F | GCHPPRGDNPPVTCKQDSDCLAGCVCGPNGFCG | (SEQ ID NO: 14) |
| 2.3A | GCPEPRGDNPPPSCKQDSDCRAGCVCGPNGFCG | (SEQ ID NO: 15) |
| 2.3B | GCLPPRGDNPPPSCKQDSDCQAGCVCGPNGFCG | (SEQ ID NO: 16) |
| 2.30 | GCHLGRGDWAPVGCKQDSDCPAGCVCGPNGFCG | (SEQ ID NO: 17) |
| 2.3D | GCNVGRGDWAPSECKQDSDCPAGCVCGPNGFCG | (SEQ ID NO: 18) |
| 2.3E | GCFPGRGDWAPSSCKQDSDCRAGCVCGPNGFCG | (SEQ ID NO: 19) |
| 2.3F | GCPLPRGDNPPTECKQDSDCQAGCVCGPNGFCG | (SEQ ID NO: 20) |
| 2.3G | GCSEARGDNPRLSCKQDSDCRAGCVCGPNGFCG | (SEQ ID NO: 21) |
| 2.3H | GCLLGRGDWAPEACKQDSDCRAGCVCGPNGFCG | (SEQ ID NO: 22) |
| 2.31 | GCHVGRGDWAPLKCKQDSDCQAGCVCGPNGFCG | (SEQ ID NO: 23) |
| 2.3J | GCVRGRGDWAPPSCKQDSDCPAGCVCGPNGFCG | (SEQ ID NO: 24) |
| 2.4A | GCLGGRGDWAPPACKQDSDCRAGCVCGPNGFCG | (SEQ ID NO: 25) |
| 2.40 | GCFVGRGDWAPLTCKQDSDCQAGCVCGPNGFCG | (SEQ ID NO: 26) |
| 2.4D | GCPVGRGDWSPASCKQDSDCRAGCVCGPNGFCG | (SEQ ID NO: 27) |
| 2.4E | GCPRPRGDNPPLTCKQDSDCLAGCVCGPNGFCG | (SEQ ID NO: 28) |
| 2.4F | GCYQGRGDWSPSSCKQDSDCPAGCVCGPNGFCG | (SEQ ID NO: 29) |
| 2.4G | GCAPGRGDWAPSECKQDSDCQAGCVCGPNGFCG | (SEQ ID NO: 30) |
| 2.4J | GCVQGRGDWSPPSCKQDSDCPAGCVCGPNGFCG | (SEQ ID NO: 31) |
| 2.5A | GCHVGRGDWAPEECKQDSDCQAGCVCGPNGFCG | (SEQ ID NO: 32) |
| 2.50 | GCDGGRGDWAPPACKQDSDCRAGCVCGPNGFCG | (SEQ ID NO: 33) |
| 2.5D | GCPQGRGDWAPTSCKQDSDCRAGCVCGPNGFCG | (SEQ ID NO: 6) |
| 2.5F | GCPRPRGDNPPLTCKQDSDCLAGCVCGPNGFCG | (SEQ ID NO: 7) |
| 2.5H | GCPQGRGDWAPEWCKQDSDCPAGCVCGPNGFCG | (SEQ ID NO: 34) |
| 2.5J | GCPRGRGDWSPPACKQDSDCQAGCVCGPNGFCG | (SEQ ID NO: 35) |

In some embodiments, the knottin peptide of a conjugate of the present disclosure is an integrin-binding AgRP-based knottin peptide as set forth in Table 2.

TABLE 2
Example AgRP Intedrin-Binding Knottin Peptides

| Clone | Loop 4 sequence |
|---|---|
| 7A (5E) (SEQ ID NO: 36) | GCVRLHESCLGQQVPCCDPAATCYC |
| | SGRGDNDLVCYCR |
| 7B (SEQ ID NO: 37) | GCVRLHESCLGQQVPCCDPAATCYC |
| | KGRGDARLQCYCR |
| 7E (SEQ ID NO: 38) | GCVRLHESCLGQQVPCCDPAATCYC |
| | VGRGDDNLKCYCR |
| 7J (6B) (SEQ ID NO: 39) | GCVRLHESCLGQQVPCCDPAATCYC |
| | EGRGDRDMKCYCR |
| 7C (SEQ ID NO: 40) | GCVRLHESCLGQQVPCCDPAATCYC |
| | YGRGDNDLRCYCR |

In some embodiments, the knottin peptide includes one or more unnatural amino acids. Such one or more unnatural amino acids may find use, e.g., to facilitate conjugation of the drug to the knottin peptide. Unnatural amino acids which find use, e.g., for preparing the conjugates of the present disclosure, include those having a functional group selected from an azide, alkyne, alkene, amino-oxy, hydrazine, aldehyde, nitrone, nitrile oxide, cyclopropene, norbornene, iso-cyanide, aryl halide, and boronic acid functional group. Unnatural amino acids which may be incorporated into a knottin peptide of a knottin-drug conjugate of the present disclosure, which unnatural amino acid may be selected to provide a functional group of interest are known and described in, e.g., Maza et al. (2015) Bioconjug. Chem. 26(9):1884-9; Patterson et al. (2014) ACS Chem. Biol. 9:592-605; Adumeau et al. (2016) Mol. Imaging Biol. (2):153-65; and elsewhere.

The manner in which the knottin peptide having an engineered loop that binds to a cell surface molecule is developed may vary. Rational and combinatorial approaches have been used to engineer knottins with novel molecular recognition properties. For example, a library of knottin proteins may be created and screened, e.g., by bacterial display, phage display, yeast surface display, fluorescence-activated cell sorting (FACS), and/or any other suitable screening method.

Yeast surface display is a powerful combinatorial technology that has been used to engineer proteins with novel molecular recognition properties, increased target binding affinity, proper folding, and improved stability. In this platform, libraries of protein variants are generated and screened in a high-throughput manner to isolate mutants with desired biochemical and biophysical properties. Yeast surface display has proven to be a successful combinatorial method for engineering knottins with altered molecular recognition. Yeast surface display benefits from quality control mechanisms of the eukaryotic secretory pathway, chaperone-assisted folding, and efficient disulfide bond formation.

One example approach for developing a knottin peptide having an engineered loop that binds to a cell surface molecule of interest involves genetically fusing the peptide to the yeast mating agglutinin protein Aga2p, which is attached by two disulfide binds to the yeast cell wall protein Aga1p. This Aga2p-fusion construct, and a chromosomally integrated Aga1p expression cassette, may be expressed under the control of a suitable promoter, such as a galactose-inducible promoter. N- or C-terminal epitope tags may be included to measure cell surface expression levels by flow cytometry using fluorescently labeled primary or secondary antibodies. This construct represents the most widely used display format, where the N-terminus of the knottin (or other protein to be engineered) is fused to Aga2, but several alternative variations of the yeast surface display plasmid have been described and may be employed to develop a knottin peptide for use in a conjugate of the present disclosure. One of the benefits of this screening platform over panning-based methods used with phage or mRNA display is that two-color FACS can be used to quantitatively discriminate clones that differ by as little as two-fold in binding affinity to the desired target.

To selectively mutate knottin loop regions at the DNA level, degenerate codons can be introduced by oligonucleotide assembly using, e.g., overlap extension PCR. Next, the genetic material may be amplified using flanking primers with sufficient overlap with the yeast display vector for homologous recombination in yeast. This assembly and amplification method allow knottin libraries to be created at relatively low cost and effort. Synthetic oligonucleotide libraries and recent methods have been developed that allow defined control over library composition.

In certain aspects, a display library (e.g., a yeast display library) is screened for binding to the cell surface molecule of interest by FACS. When screening knottin libraries by FACS, an enriched pool of binders generally emerges in 4-7 rounds of sorting. Two-color FACS may be used for library screening, where one fluorescent label can be used to detect the c-myc epitope tag and the other to measure interaction of the knottin mutant against the binding target of interest. Different instrument lasers and/or filter sets can be used to measure excitation and emission properties of the two fluorophores at single-cell resolution. This enables yeast expression levels to be normalized with binding. That is, a knottin that exhibits poor yeast expression but binds a high amount of a target can be distinguished from a knottin that is expressed at high levels but binds weakly to a target. Accordingly, a two-dimensional flow cytometry plot of expression versus binding will result in a diagonal population of yeast cells that bind to target antigen. High-affinity binders can be isolated using library sort gates. Alternatively, in an initial sort round it could be useful to clear the library of undesired clones that do not express full-length. The target used in the screening is structurally and functionally relevant for the final application, e.g., mimics the cell surface molecule of interest.

Following enrichment of knottin libraries for binders against the cell surface molecule of interest, the yeast plasmids are recovered and sequenced. Additional rounds of FACS can be performed under increased sorting stringency. The binding affinities or kinetic off-rates of individual yeast-displayed knottin clones may then be measured.

Once knottin peptides having an engineered loop that binds to the cell surface molecule of interest have been identified by surface display (e.g., yeast surface display), the engineered knottins may be produced using a suitable method. The small size of knottins makes them amenable to production by both chemical synthesis and recombinant expression. According to certain embodiments, the knottin peptide may be produced by solid phase peptide synthesis followed by in vitro folding. Chemical synthesis permits facile incorporation of unnatural amino acids or other chemical handles into knottin peptides.

Knottin peptides not fused to large heterologous domains are readily synthesized using solid phase peptide chemistry on an automated synthesizer. For example, standard 9-fluorenylmethyloxycarbonyl (Fmoc)-based solid phase peptide chemistry may be employed. The linear peptide may then be folded under conditions that promote oxidation of cysteine side chain thiols to form disulfide bonds, followed by purification, e.g., by reversed-phase high-performance liquid chromatography (RP-HPLC).

In certain aspects, the knottin peptide or a fusion protein that includes the knottin peptide fused to an antibody subunit or fragment thereof is produced using a recombinant DNA approach. Any suitable strategy for producing the knottin peptide or fusion protein using recombinant methods in a variety of host cell types may be employed. For example, functional knottins have been produced with barnase as a genetic fusion partner, which promotes folding in the E. coli periplasmic space and serves as a useful purification handle. According to certain embodiments, the engineered knottin peptide is expressed in yeast. The yeast strain Pichia pastoris, for example, has been successfully employed to produce 2-10 mg/L of purified engineered knottins. The yeast expression construct may encode one or more tags (e.g., a C-terminal hexahistadine tag for purification by, e.g., metal chelating chromatography (Ni-NTA)). Size exclusion chromatography may then be used to remove aggregates, misfolded multimers, and the like.

Aspects of the present disclosure include nucleic acids that encode the knottin peptides and fusion proteins employed in the conjugates of the present disclosure. That is, provided are nucleic acids that encode any of the knottin peptides and fusion proteins described herein having an engineered loop that binds to a cell surface molecule of interest. In certain aspects, such a nucleic acid is present in an expression vector. The expression vector includes a promoter operably linked to the nucleic acid encoding the knottin peptide, the promoter being selected based on the type of host cell selected to express the knottin peptide. Also provided are host cells that include any of the knottin peptide-encoding nucleic acids of the present disclosure, as well as any expression vectors including the same.

Methods are available for measuring the affinity of knottins for molecules expressed on the surface of cells (e.g., cancer cells, such as mammalian cancer cells) using direct binding or competition binding assays. In a direct binding assay, an equilibrium binding constant (K_{D}) may be measured using a knottin conjugated to a fluorophore or radioisotope, or a knottin that contains an N- or C-terminal epitope tag for detection by a labeled antibody. If labels or tags are not feasible or desired, a competition binding assay can be used to determine the half-maximal inhibitory concentration (IC₅₀), the amount of unlabeled knottin at which 50% of the maximal signal of the labeled competitor is detectable. A K_{D} value can then be calculated from the measured IC₅₀ value. Ligand depletion will be more pronounced when measuring high-affinity interactions over a lower concentration range, and can be avoided or minimized by decreasing the number of cells added in the experiment or by increasing the binding reaction volumes.

In certain aspects, the knottin peptide or fusion protein has an equilibrium binding constant (K_{D}) for the cell surface molecule of from about 0.01 nM to 100 nM, such as from about 0.025 nM to 75 nM, about 0.05 nM to 50 nm, about 0.075 nM to 25 nM, or from about 0.1 nM to 10 nM. In some embodiments, the knottin peptide or fusion protein has an equilibrium binding constant (K_{D}) for the cell surface molecule of from about 0.1 nM to 10 nM. In some embodiments, the knottin peptide or fusion protein has an equilibrium binding constant (K_{D}) for the cell surface molecule of about 0.1 nM. In some embodiments, the knottin peptide or fusion protein has an equilibrium binding constant (K_{D}) for the cell surface molecule of about 0.5 nM. In some embodiments, the knottin peptide or fusion protein has an equilibrium binding constant (K_{D}) for the cell surface molecule of about 1 nM. In some embodiments, the knottin peptide or fusion protein has an equilibrium binding constant (K_{D}) for the cell surface molecule of about 5 nM. In some embodiments, the knottin peptide or fusion protein has an equilibrium binding constant (K_{D}) for the cell surface molecule of about 10 nM.

Detailed guidance and specific protocols for engineering knottins by yeast surface display technology, including knottin library construction and screening, as well as knottin production by chemical synthesis and recombinant expression, and further for cell binding assays to measure the affinity of knottins to molecules (e.g., receptors) expressed on the surface of cells using direct binding or competition binding assays, are described in Moore, S. and Cochran, J. (2012) Engineering Knottins as Novel Binding Agents, Methods in Enzymology, 503, 223-251.

Knottin-Drug Conjugates

Existing targeting agents rely nearly exclusively on high molecular weight (MW) proteins, namely antibodies, with the potential of alternative low MW targeting proteins left unexplored. Despite over ˜100 antibody-drug conjugate (ADC) candidates in clinical development, only four ADCs have been approved by the FDA (Mylotarg®, Adcetris®, Kadcyla®, and Besponsa®). Significant barriers still impede the clinical success of this class of targeted therapeutics. More than 55 ADC candidates have failed clinical trials and at least 23 of these failures were due to poor therapeutic window. ADCs approved or in late-stage clinical development are substantially skewed towards hematological cancers, and have a drastic reduction in clinical success against solid tumors. The large molecular weight immunoglobulin G (IgG) portion of an antibody is faced with physical challenges to efficient extravasation out of blood vessels and diffusion throughout solid tumors. Moreover, the extended circulatory half-life of ADCs, originally assumed to be wholly beneficial by allowing increased absolution drug accumulation in tumors, also contributes to off-target toxicity by increasing healthy tissue exposure to both intact conjugate and prematurely released drug.

Low MW targeting agents would offer a different pharmacokinetic profile and have potential to overcome historical limitations of antibody-drug conjugates including limited efficacy against solid tumors and exclusion from targeting brain tumors due to an inability to cross the blood-brain-barrier. Despite the theoretical advantages of low MW targeting proteins, there exists a widespread assumption that this class of targeting agent would be ineffective due to rapid systemic clearance or toxicity. As demonstrated herein, however, the low MW targeting agents of the present disclosure exhibit unexpectedly high efficacy, e.g., anti-tumor efficacy.

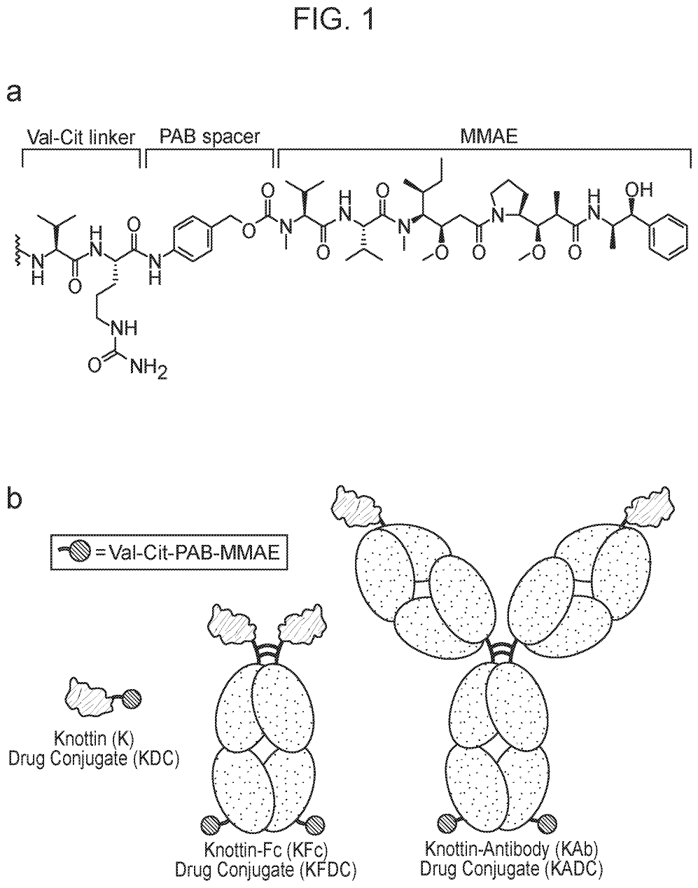

In one example of a low MW targeting agent, provided are conjugates that include a knottin peptide including an engineered loop that binds to a cell surface molecule (including but not limited to any of the knottin peptides described above), and an anti-microtubule agent conjugated to the knottin peptide via a linker. A non-limiting example of such a conjugate is schematically illustrated in FIG. 1, panel B (right—sometimes referred to herein as knottin drug conjugate, or “KDC”).

As used herein, an “anti-microtubule agent” is an agent that prevents the formation of, and/or disrupts, microtubules. Microtubules are dynamically instable cytoskeletal protein polymers that are critical for numerous essential mammalian cell functions including cellular division, differentiation, transport, and motility. The biological function of microtubules is regulated by and dependent upon the depolymerization and repolymerization of αβ-tubulin heterodimers. The non-equilibrium polymerization of microtubules can be reversed via a disassembly process termed ‘catastrophe’, which releases GDP-tubulin. Conversely, the exchange of GDP to GTP regenerates the active form of tubulin, which is polymerized via GTP hydrolysis. GTP-tubulin interactions therefore play an important role in the cellular regulation of microtubule dynamics. Because of their central importance to various cellular functions, tubulin dimers and the microtubule cytoskeleton are targets for a surfeit of cytotoxic agents that disrupt mitosis.

In certain embodiments, the anti-microtubule agent conjugated to the knottin peptide is a tubulin inhibitor. According to some embodiments, the tubulin inhibitor is an auristatin. Non-limiting examples of auristatins that find use in the knottin conjugates of the present disclosure include auristatin E and auristatin F. In certain embodiments the auristatin is monomethylauristatin F (MMAF). According to some embodiments, the auristatin is monomethylauristatin E (MMAE—the structure of which is provided in FIG. 1, panel a). MMAE is a dolastatin 10 derivative with improved activity over its parent compound as well as increased toxicity. MMAE comprises the four amino acids: monomethylvaline (MeVal), valine (Val), dolaisoleuine (Dil) and dolaproine (Dap), and the carboxy-terminal amine norephedrine. Many tubulin inhibitors are loosely classified by the general region of the tubulin dimer to which they bind. MMAE falls into the category of vinca-site antimitotics. Vinca-site antimitotics typically bind at the interface between two longitudinally aligned tubulin dimers, between a β₁-tubulin subunit and the adjacent α₂-tubulin subunit, a domain targeted by classical alkaloid microtubule-destabilizing agents such as vinblastine and eribulin. Biophysical experiments and crystal structures have been used to confirm that MMAE binds a structurally distinct regions from the vinca-site called the peptide site. MMAE is a potent tubulin inhibitor that binds tightly to the peptide site near the α/β tubulin interface and effectively blocks GTP hydrolysis and thereby polymerization. Moreover, MMAE has more recently been shown to induce an ordered, helical structure in the naturally disordered β-tubulin M-loop.⁹⁵ This conformational change further disrupts microtubule formation by sterically blocking microtubule polymerization.

In the knottin-drug conjugates of the present disclosure, the anti-microtubule agent is conjugated to the knottin peptide via a linker. Non-limiting examples of linkers that may be employed in the conjugates of the present disclosure include ester linkers, amide linkers, maleimide or maleimide-based linkers; valine-citrulline linkers; hydrazone linkers; N-succinimidyl-4-(2-pyridyldithio)butyrate (SPDB) linkers; Succinimidyl-4-(N-maleimidomethyl)cyclohexane-1-carboxylate (SMCC) linkers; vinylsulfone-based linkers; linkers that include polyethylene glycol (PEG), such as, but not limited to tetraethylene glycol; linkers that include propanoic acid; linkers that include caproleic acid, and linkers including any combination thereof. In certain aspects, the linker is a chemically-labile linker, such as an acid-cleavable linker that is stable at neutral pH (bloodstream pH 7.3-7.5) but undergoes hydrolysis upon internalization into the mildly acidic endosomes (pH 5.0-6.5) and lysosomes (pH 4.5-5.0) of a target cell (e.g., a cancer cell). Chemically-labile linkers include, but are not limited to, hydrazone-based linkers, oxime-based linkers, carbonate-based linkers, ester-based linkers, etc. According to certain embodiments, the linker is an enzyme-labile linker, such as an enzyme-labile linker that is stable in the bloodstream but undergoes enzymatic cleavage upon internalization into a target cell, e.g., by a lysosomal protease (such as cathepsin or plasmin) in a lysosome of the target cell (e.g., a cancer cell). Enzyme-labile linkers include, but are not limited to, linkers that include peptidic bonds, e.g., dipeptide-based linkers such as valine-citrulline linkers, such as a maleimidocaproyl-valine-citruline-p-aminobenzyl (MC-vc-PAB) linker, a valyl-alanyl-para-aminobenzyloxy (Val-Ala-PAB) linker, and the like. Chemically-labile linkers, enzyme-labile, and non-cleavable linkers are known and described in detail, e.g., in Ducry & Stump (2010) Bioconjugate Chem. 21:5-13.

In certain embodiments, a conjugate of the present disclosure includes a linker that includes a valine-citrulline dipeptide, a valine-alanine dipeptide, or both. According to some embodiments, the linker is a valine-citruline-paraaminobenzyloxy (Val-Cit-PAB) linker (schematically illustrated in FIG. 1 panel a). In certain embodiments, the linker is a valylalanylparaaminobenzyloxy (Val-Ala-PAB) linker.

Knottin-Antibody Subunit Conjugates

Aspects of the present disclosure further include knottin-antibody subunit conjugates. Such conjugates include a fusion protein that includes a knottin peptide including an engineered loop that binds to a cell surface molecule (including but not limited to any of the knottin peptides described above), fused to an antibody subunit or fragment thereof. Such conjugates further include a drug conjugated to the fusion protein via a linker. In some embodiments, provided are dimers of such conjugates, where the antibody subunits or fragments thereof dimerize (e.g., via disulfide bridges at a hinge region (if present), or the like) to form dimerized knottin drug conjugates.

According to some embodiments, the antibody subunit or fragment thereof is an antibody heavy chain or fragment thereof. In certain embodiments, the antibody heavy chain or fragment thereof includes a γ, α, δ, ε, or μ antibody heavy chain or fragment thereof. According to some embodiments, the antibody heavy chain or fragment thereof is an IgG heavy chain or fragment thereof, e.g., a human IgG1 heavy chain or fragment thereof. In certain embodiments, the antibody heavy chain or fragment thereof comprises a heavy chain variable region (V_{H}). Such an antibody heavy chain or fragment thereof may further include a heavy chain constant region or fragment thereof. For example, when a heavy chain constant region or fragment thereof is included in the fusion protein, the antibody heavy chain constant region or fragment thereof may include one or more of a C_{H}1 domain, C_{H}2 domain, and/or C_{H}3 domain. According to some embodiments, the antibody heavy chain or fragment thereof is a full-length antibody heavy chain—that is, an antibody heavy chain that includes a V_{H}, a C_{H}1 domain, a C_{H}2 domain, and a C_{H}3 domain.

In certain embodiments, the antibody subunit or fragment thereof is an antibody heavy chain or fragment thereof that does not include a V_{H}. Such an antibody heavy chain or fragment thereof may include, consist essentially of, or consist of an Fc region. A non-limiting example of an Fc region which may be included in any of the heavy chain (or fragment thereof)-containing conjugates of the present disclosure has the following amino acid sequence:

TABLE 3
Amino Acid Sequence of an Example Human Fc Region

| | Amino Acid Sequence |
|---|---|
| Example Human | DKTHTCPPCPAPELLGGPSVFLFPPKPKDTLMIS |
| Fc Region | RTPEVTCVVVDVSHEDPEVKFNWYVDGVEVHNAK |
| (SEQ ID NO: 41) | TKPREEQYNSTYRVVSVLTVLHQDWLNGKEYKCK |
| | VSNKALPAPIEKTISKAKGQPREPQVYTLPPSRD |
| | ELTKNQVSLTCLVKGFYPSDIAVEWESNGQPENN |
| | YKTTPPVLDSDGSFFLYSKLTVDKSRWQQGNVFS |
| | CSVMHEALHNHYTQKSLSLSPGK |

When a conjugate of the present disclosure includes a knottin peptide fused to an antibody heavy chain or fragment thereof, the knottin peptide may be fused to the N-terminus of the antibody heavy chain or fragment thereof. Alternatively, the knottin peptide may be fused to the C-terminus of the antibody heavy chain or fragment thereof.

For the conjugates that include a knottin peptide fused to an antibody heavy chain or fragment thereof, the drug may be conjugated to the knottin peptide portion of the fusion protein. Alternatively, the drug may be conjugated to the antibody heavy chain or fragment thereof portion of the fusion protein. For example, when the antibody heavy chain or fragment thereof includes, consists essentially of, or consists of Fc region, the drug may be conjugated to the Fc region. In these embodiments, the drug may be conjugated to the C_{H}1 domain of the Fc region, the C_{H}2 domain of the Fc region, or the C_{H}3 domain of the Fc region, e.g., at or near the C-terminus of the Fc region.

According to some embodiments, the antibody subunit or fragment thereof is an antibody light chain or fragment thereof. In certain embodiments, the antibody light chain or fragment thereof includes a kappa (κ) light chain or fragment thereof or a lambda (λ) light chain or fragment thereof. According to some embodiments, the antibody light chain or fragment thereof includes a light chain variable region (V_{L}). Such an antibody light chain or fragment thereof may further include an antibody light chain constant region (C_{L}) or fragment thereof. In certain embodiments, the antibody light chain or fragment thereof is a full-length antibody light chain—that is, an antibody light chain that includes a V_{L} and a C_{L}.

When a conjugate of the present disclosure includes a knottin peptide fused to an antibody light chain or fragment thereof, the knottin peptide may be fused to the N-terminus of the antibody light chain or fragment thereof. Alternatively, the knottin peptide may be fused to the C-terminus of the antibody light chain or fragment thereof.

For the conjugates that include a knottin peptide fused to an antibody light chain or fragment thereof, the drug may be conjugated to the knottin peptide portion of the fusion protein. Alternatively, the drug may be conjugated to the antibody light chain or fragment thereof portion of the fusion protein. For example, the drug may be conjugated to a V_{L} (if present) or a C_{L} (if present), e.g., at or near the C-terminus of a C_{L}.

In certain embodiments, when a conjugate of the present disclosure includes a knottin peptide fused to an antibody subunit or fragment thereof that includes a heavy chain variable region (V_{H}) or a light chain variable region (V_{L}), the V_{H} or V_{L} does not bind to an antigen on the surface of the cell that includes the cell surface molecule to which the engineered loop of the knottin binds. In certain embodiments, when a conjugate of the present disclosure includes a knottin peptide fused to an antibody subunit or fragment thereof that includes a heavy chain variable region (V_{H}) or a light chain variable region (V_{L}), the knottin is fused to the V_{H} or V_{L} such that the V_{H} or V_{L} does not bind antigen.

The drug employed in the conjugates that include a knottin peptide fused to an antibody subunit or fragment thereof may be any suitable agent and will vary depending on the application for which the conjugate is employed, e.g., killing, prevention of cell proliferation, etc. Non-limiting examples of drugs that may be included in the conjugates include toxins, fragments of toxins, antiproliferative agents, antineoplastic agents, and the like. In certain aspects, the conjugate includes a drug that reduces the function of a target cell/tissue by inhibiting cell proliferation and/or killing the cell/tissue. Such agents may vary and include cytostatic agents and cytotoxic agents, e.g., an agent capable of killing a target cell tissue with or without being internalized into a target cell.

In some embodiments, the drug of the conjugate is a cytotoxic agent, such as a cytotoxic agent selected from an enediyne, a lexitropsin, a duocarmycin, a taxane, a puromycin, a dolastatin, a maytansinoid, and a vinca alkaloid. According to certain embodiments, the cytotoxic agent is paclitaxel, docetaxel, CC-1065, CPT-11 (SN-38), topotecan, doxorubicin, morpholino-doxorubicin, rhizoxin, cyanomorpholino-doxorubicin, dolastatin-10, echinomycin, combretastatin, calicheamicin, maytansine, maytansine DM1, maytansine DM4, DM-1, an auristatin or other dolastatin derivatives, such as auristatin E or auristatin F, AEB (AEB-071), AEVB (5-benzoylvaleric acid-AE ester), AEFP (antibody-endostatin fusion protein), MMAE (monomethylauristatin E), MMAF (monomethylauristatin F), pyrrolobenzodiazepines (PBDs), eleutherobin, netropsin, or any combination thereof.

According to certain embodiments, the drug of the conjugate is a protein toxin selected from hemiasterlin and hemiasterlin analogs such as HTI-286 (e.g., see U.S. Pat. No. 7,579,323; WO 2004/026293; and U.S. Pat. No. 8,129,407, the full disclosures of which are incorporated herein by reference), abrin, brucine, cicutoxin, diphtheria toxin, batrachotoxin, botulism toxin, shiga toxin, endotoxin, Pseudomonas exotoxin, Pseudomonas endotoxin, tetanus toxin, pertussis toxin, anthrax toxin, cholera toxin, falcarinol, fumonisin BI, fumonisin B2, afla toxin, maurotoxin, agitoxin, charybdotoxin, margatoxin, slotoxin, scyllatoxin, hefutoxin, calciseptine, taicatoxin, calcicludine, geldanamycin, gelonin, lotaustralin, ocratoxin A, patulin, ricin, strychnine, trichothecene, zearlenone, and tetradotoxin. Enzymatically active toxins and fragments thereof which may be employed include diphtheria A chain, non-binding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin and the tricothecenes.

In certain embodiments, the drug of the conjugate is selected from an anti-microtubule agent, a tubulin inhibitor, an auristatin, an auristatin E, an auristatin F, monomethylauristatin E (MMAE), monomethylauristatin F (MMAF), an auristatin W derivative, maytansine, a maytansine derivative, N^{2′}-deacetyl-N^{2′}-(3-mercapto-1-oxopropyl)-maytansine (DM1), ravtansine (DM4), pyrrolobenzodiazepine (PBD), calicheamicin, duocarmycin, doxorubicin, SN-38, DXd, liposomal doxorubicin, and tubulysin.

According to some embodiments, the drug of the conjugate is a nucleoside drug. Such a nucleoside drug may be a nucleoside analogue. Non-limiting examples of nucleoside analogues that may be employed include gemcitabine, cytarabine, troxacitabine, decitabine, cladribine, fludarabine, clofarabine, and 2′-C-cyano-2′-deoxy-1-β-D-arabino-pentofuranosylcytosine (CNDAC).

Any suitable linker may be employed in the conjugates that include a knottin peptide fused to an antibody heavy chain or fragment thereof. Non-limiting examples of such linkers are described in the preceding section relating to knottin-drug conjugates, which description is incorporated but not reiterated herein for purposes of brevity. In certain embodiments, a conjugate that includes a knottin peptide fused to an antibody heavy chain or fragment thereof includes a linker that includes a valine-citrulline dipeptide, a valine-alanine dipeptide, or both. According to some embodiments, the linker is a valine-citruline-paraaminobenzyloxy (Val-Cit-PAB) linker (schematically illustrated in FIG. 1 panel a). In certain embodiments, the linker is a valylalanylparaaminobenzyloxy (Val-Ala-PAB) linker.

Methods of Making Conjugates

Aspects of the present disclosure further include methods of making conjugates. Such methods including conjugating the drug to the knottin peptide in the case of knottin-drug conjugates, or conjugating the drug to the knottin peptide or antibody subunit or fragment thereof in the case of the knottin-antibody subunit conjugates. In some embodiments, the methods include site-specifically conjugating the drug to the knottin peptide or antibody subunit or fragment thereof. For example, the conjugating may include site-specifically conjugating the drug to a pre-selected amino acid of the knottin peptide or antibody subunit or fragment thereof. In certain aspects, the pre-selected amino acid is at the N-terminus or C-terminus of the knottin peptide or antibody subunit or fragment thereof. In other aspects, the pre-selected amino acid is internal to the knottin peptide or antibody subunit or fragment thereof—that is, between the N-terminal and C-terminal amino acid of the knottin peptide or antibody subunit or fragment thereof. In some embodiments, the pre-selected amino acid is a non-natural amino acid. Non-limiting examples of non-natural amino acids which may be provided to the knottin peptide or antibody subunit or fragment thereof to facilitate conjugation include those having a functional group selected from an azide, alkyne, alkene, amino-oxy, hydrazine, aldehyde (e.g., formylglycine, e.g., SMARTag™ technology from Catalent Pharma Solutions), nitrone, nitrile oxide, cyclopropene, norbornene, iso-cyanide, aryl halide, and boronic acid functional group. Unnatural amino acids which may be incorporated and selected to provide a functional group of interest are known and described in, e.g., Maza et al. (2015) Bioconjug. Chem. 26(9):1884-9; Patterson et al. (2014) ACS Chem. Biol. 9:592-605; Adumeau et al. (2016) Mol. Imaging Biol. (2):153-65; and elsewhere.

Numerous strategies are available for conjugating the drug and knottin peptide or antibody subunit or fragment thereof through a linker. For example, the drug may be derivatized by covalently attaching the linker to the drug, where the linker has a functional group capable of reacting with a “chemical handle” on the knottin peptide or antibody subunit or fragment thereof. Also by way of example, the knottin peptide or antibody subunit or fragment thereof may be derivatized by covalently attaching the linker to the knottin peptide or antibody subunit or fragment thereof, where the linker has a functional group capable of reacting with a “chemical handle” on the drug. The functional group on the linker may vary and may be selected based on compatibility with the chemical handle on the drug or knottin peptide or antibody subunit or fragment thereof. According to one embodiment, the chemical handle is provided by incorporation of an unnatural amino acid having the chemical handle into the drug or knottin peptide or antibody subunit or fragment thereof. In some embodiments, conjugating the drug and knottin peptide or antibody subunit or fragment thereof is by copper-free, strain-promoted cycloaddition, alkyne-azide cycloaddition, or the like.

### Compositions

As summarized above, the present disclosure provides compositions. The compositions may include any of the conjugates of the present disclosure, including any of the conjugates described in the Conjugates section above, which is incorporated but not reiterated herein for purposes of brevity.

In certain aspects, the compositions include a conjugate of the present disclosure present in a liquid medium. The liquid medium may be an aqueous liquid medium, such as water, a buffered solution, and the like. One or more additives such as a salt (e.g., NaCl, MgCl₂, KCl, MgSO₄), a buffering agent (a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.), a protease inhibitor, glycerol, and the like may be present in such compositions.

Pharmaceutical compositions are also provided. The pharmaceutical compositions include any of the conjugates of the present disclosure, and a pharmaceutically-acceptable carrier. The pharmaceutical compositions generally include a therapeutically effective amount of the conjugate. By “therapeutically effective amount” is meant a dosage sufficient to produce a desired result, e.g., an amount sufficient to effect beneficial or desired therapeutic (including preventative) results, such as a reduction in cellular proliferation in an individual having a cell proliferative disorder (e.g., cancer) associated with the cell surface molecule to which the engineered loop binds, etc. An effective amount may be administered in one or more administrations.

A conjugate of the present disclosure can be incorporated into a variety of formulations for therapeutic administration. More particularly, the conjugate can be formulated into pharmaceutical compositions by combination with appropriate pharmaceutically acceptable excipients or diluents, and may be formulated into preparations in solid, semi-solid, liquid or gaseous forms, such as tablets, capsules, powders, granules, ointments, solutions, injections, inhalants and aerosols.

Formulations of the conjugates of the present disclosure suitable for administration to an individual (e.g., suitable for human administration) are generally sterile and may further be free of detectable pyrogens or other contaminants contraindicated for administration to an individual according to a selected route of administration.

In pharmaceutical dosage forms, the conjugate can be administered alone or in appropriate association, as well as in combination, with other pharmaceutically-active compounds. The following methods and excipients are merely examples and are in no way limiting.

For oral preparations, the conjugate can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The conjugates can be formulated into preparations for injection by dissolving, suspending or emulsifying them in an aqueous or non-aqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The pharmaceutical composition may be in a liquid form, a lyophilized form or a liquid form reconstituted from a lyophilized form, where the lyophilized preparation is to be reconstituted with a sterile solution prior to administration. The standard procedure for reconstituting a lyophilized composition is to add back a volume of pure water (typically equivalent to the volume removed during lyophilization); however solutions comprising antibacterial agents may be used for the production of pharmaceutical compositions for parenteral administration.

An aqueous formulation of the conjugate may be prepared in a pH-buffered solution, e.g., at pH ranging from about 4.0 to about 8.0, such as from about 4.5 to about 7.5, e.g., from about 5.0 to about 7.0. Examples of buffers that are suitable for a pH within this range include phosphate-, histidine-, citrate-, succinate-, acetate-buffers and other organic acid buffers. The buffer concentration can be from about 1 mM to about 100 mM, or from about 5 mM to about 50 mM, depending, e.g., on the buffer and the desired tonicity of the formulation.

### Methods

As summarized above, also provides are methods of using the conjugates of the present disclosure. In some embodiments, the methods including using any of the conjugates described in the Conjugates section above, which is incorporated but not reiterated herein for purposes of brevity.

In some embodiments, provided are methods that include administering to an individual in need thereof a therapeutically effective amount of any of the conjugates or any of the pharmaceutical compositions of the present disclosure. In some embodiments, the individual has cancer and the engineered loop binds to a cell surface molecule on cancer cells present in the individual. Accordingly, aspects of the present disclosure include methods of treating cancer by administering to an individual having cancer a therapeutically effective amount of any of the conjugates or any of the pharmaceutical compositions of the present disclosure. A variety of individuals are treatable according to the subject methods. Generally such subjects are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In some embodiments, the individual is a human. In some embodiments, the individual is an animal model, such as a mouse model.

In some embodiments, an effective amount of the conjugate (or pharmaceutical composition including same) is an amount that, when administered alone (e.g., in monotherapy) or in combination (e.g., in combination therapy) with one or more additional therapeutic agents, in one or more doses, is effective to reduce the symptoms of a medical condition of the individual (e.g., cancer, etc.) by at least about 5%, at least about 10%, at least about 15%, at least about 20%, at least about 25%, at least about 30%, at least about 40%, at least about 50%, at least about 60%, at least about 70%, at least about 80%, at least about 90%, or more, compared to the symptoms in the individual in the absence of treatment with the conjugate or pharmaceutical composition.

In some embodiments, the individual has a cancer characterized by the presence of a solid tumor, a semi-solid tumor, a primary tumor, a metastatic tumor, or the like. In some embodiments, the individual has a cancer selected from breast cancer, melanoma, lung cancer, colorectal cancer, prostate cancer, glioma, bladder cancer, endometrial cancer, kidney cancer, leukemia (e.g., acute myeloid leukemia (AML)) liver cancer (e.g., hepatocellular carcinoma (HCC), such as primary or recurrent HCC), non-Hodgkin lymphoma, pancreatic cancer, thyroid cancer, any combinations thereof, and any sub-types thereof.

By “treat”, “treating” or “treatment” is meant at least an amelioration of the symptoms associated with the medical condition (e.g., cell proliferative disorder, e.g., cancer) of the individual, where amelioration is used in a broad sense to refer to at least a reduction in the magnitude of a parameter, e.g. symptom, associated with the medical condition being treated. As such, treatment also includes situations where the medical condition, or at least symptoms associated therewith, are completely inhibited, e.g., prevented from happening, or stopped, e.g., terminated, such that the individual no longer suffers from the medical condition, or at least the symptoms that characterize the medical condition.

The conjugate or pharmaceutical composition may be administered to the individual using any available method and route suitable for drug delivery, including in vivo and ex vivo methods, as well as systemic and localized routes of administration. Conventional and pharmaceutically acceptable routes of administration include intranasal, intramuscular, intra-tracheal, subcutaneous, intradermal, topical application, ocular, intravenous, intra-arterial, nasal, oral, and other enteral and parenteral routes of administration. In some embodiments, the administering is by parenteral administration. Routes of administration may be combined, if desired, or adjusted depending upon the conjugate and/or the desired effect. The conjugates or pharmaceutical compositions may be administered in a single dose or in multiple doses. In some embodiments, the conjugate or pharmaceutical composition is administered intravenously. In some embodiments, the conjugate or pharmaceutical composition is administered by injection, e.g., for systemic delivery (e.g., intravenous infusion) or to a local site.

In some embodiments, the individual has a solid tumor. In some embodiments, when the individual has a solid tumor, the methods include administering a knottin-drug conjugate (KDC) of the present disclosure to the individual. As demonstrated herein, such conjugates exhibit unexpectedly beneficial penetration into solid tumors as compared to higher molecular weight targeting agents.

In some embodiments, the individual has a cancer the treatment of which requires the conjugate to cross the blood-brain barrier (BBB). A non-limiting example of such a cancer is a brain tumor, e.g., glioblastoma, or the like. In some embodiments, when the individual has a cancer the treatment of which requires the conjugate to cross the BBB, the methods include administering a low molecular weight conjugate of the present disclosure to the individual, such as a knottin-drug conjugate (KDC) of the present disclosure.

### Kits

Aspects of the present disclosure further include kits. In some embodiments, a subject kit includes any of the conjugates of the present disclosure (including any of the conjugates described in the Conjugates section above, which is incorporated but not reiterated herein for purposes of brevity) or a pharmaceutical composition comprising same, and instructions for administering the pharmaceutical composition to an individual in need thereof.

In some embodiments, the conjugate or pharmaceutical composition is present in one or more (e.g., two or more) unit dosages. The term “unit dosage”, as used herein, refers to physically discrete units suitable as unitary dosages for human and animal subjects, each unit containing a predetermined quantity of the conjugate or composition calculated in an amount sufficient to produce the desired effect. The amount of the unit dosage depends on various factors, such as the particular conjugate employed, the effect to be achieved, and the pharmacodynamics associated with the conjugate, in the individual. In yet other embodiments, the kits may include a single multi dosage amount of the conjugate or pharmaceutical composition.

Components of the kits may be present in separate containers, or multiple components may be present in a single container.

The instructions included in the kits may be recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., portable flash drive, DVD, CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, the means for obtaining the instructions is recorded on a suitable substrate.

Notwithstanding the appended claims, the present disclosure is also defined by the following embodiments:

1. A conjugate, comprising:
a knottin peptide comprising an engineered loop that binds to a cell surface molecule; and
an anti-microtubule agent conjugated to the knottin peptide via a linker.
   2. The conjugate of embodiment 1, wherein the anti-microtubule agent is a tubulin inhibitor.
   3. The conjugate of embodiment 2, wherein the tubulin inhibitor is an auristatin.
   4. The conjugate of embodiment 3, wherein the tubulin inhibitor is an auristatin E or auristatin F.
   5. The conjugate of embodiment 4, wherein the tubulin inhibitor is monomethylauristatin E (MMAE) or monomethylauristatin F (MMAF).
   6. The conjugate of any one of embodiments 1 to 5, wherein the linker is an enzyme cleavable linker.
   7. The conjugate of embodiment 6, wherein the enzyme cleavable linker is a protease cleavable linker.
   8. The conjugate of embodiment 7, wherein the linker comprises a valine-citrulline dipeptide, a valine-alanine dipeptide, or both.
   9. The conjugate of embodiment 8, wherein the linker is a valine-citruline-paraaminobenzyloxy (Val-Cit-PAB) linker.
   10. The conjugate of embodiment 8, wherein the linker is a valylalanylparaaminobenzyloxy (Val-Ala-PAB) linker.
   11. A conjugate, comprising:
a fusion protein, comprising:
   a knottin peptide comprising an engineered loop that binds to a cell surface molecule, fused to
   an antibody subunit or fragment thereof; and
a drug conjugated to the fusion protein via a linker.
   12. The conjugate of embodiment 11, wherein the antibody subunit or fragment thereof is an antibody heavy chain or fragment thereof.
   13. The conjugate of embodiment 12, wherein the antibody heavy chain or fragment thereof comprises a γ, α, δ, ε, or μ antibody heavy chain or fragment thereof.
   14. The conjugate of embodiment 13, wherein the antibody heavy chain or fragment thereof is an IgG heavy chain or fragment thereof.
   15. The conjugate of embodiment 14, wherein the IgG heavy chain or fragment thereof is an IgG1 heavy chain or fragment thereof.
   16. The conjugate of any one of embodiments 12 to 15, wherein the antibody heavy chain or fragment thereof comprises a heavy chain variable region (V_{H}).
   17. The conjugate of embodiment 16, wherein the antibody heavy chain or fragment thereof further comprises a heavy chain constant region or fragment thereof.
   18. The conjugate of embodiment 17, wherein the antibody heavy chain constant region or fragment thereof comprises a C_{H}1 domain.
   19. The conjugate of embodiment 18, wherein the antibody heavy chain constant region or fragment thereof further comprises a C_{H}2 domain.
   20. The conjugate of embodiment 19, wherein the antibody heavy chain constant region or fragment thereof further comprises a C_{H}3 domain.
   21. The conjugate of embodiment 20, wherein the antibody heavy chain or fragment thereof is a full-length antibody heavy chain.
   22. The conjugate of any one of embodiments 12 to 15, wherein the antibody heavy chain or fragment thereof does not comprise a V_{H}.
   23. The conjugate of embodiment 22, wherein the antibody heavy chain or fragment thereof comprises an F_{c} region.
   24. The conjugate of any one of embodiments 12 to 23, wherein the knottin peptide is fused to the N-terminus of the antibody heavy chain or fragment thereof.
   25. The conjugate of any one of embodiments 12 to 23, wherein the knottin peptide is fused to the C-terminus of the antibody heavy chain or fragment thereof.
   26. The conjugate of any one of embodiments 12 to 25, wherein the drug is conjugated to the knottin peptide.
   27. The conjugate of any one of embodiments 12 to 25, wherein the drug is conjugated to the antibody heavy chain or fragment thereof.
   28. The conjugate of embodiment 27, wherein the antibody heavy chain or fragment thereof comprises an Fc region, and wherein the drug is conjugated to the Fc region.
   29. The conjugate of embodiment 28, wherein the drug is conjugated to the C_{H}3 domain of the Fc region.
   30. The conjugate of embodiment 28, wherein the drug is conjugated to the C_{H}2 domain of the Fc region.
   31. The conjugate of embodiment 11, wherein the antibody subunit or fragment thereof is an antibody light chain or fragment thereof.
   32. The conjugate of embodiment 31, wherein the antibody light chain or fragment thereof is a kappa (κ) light chain or fragment thereof.
   33. The conjugate of embodiment 31, wherein the antibody light chain or fragment thereof is a lambda (λ) light chain or fragment thereof.
   34. The conjugate of any one of embodiments 31 to 33, wherein the antibody light chain or fragment thereof comprises an antibody light chain variable region (V_{L}).
   35. The conjugate of embodiment 34, wherein the antibody light chain or fragment thereof further comprises an antibody light chain constant region (C_{L}) or fragment thereof.
   36. The conjugate of any one of embodiments 31 to 35, wherein the antibody light chain or fragment thereof is a full-length antibody light chain.
   37. The conjugate of any one of embodiments 31 to 36, wherein the knottin peptide is fused to the N-terminus of the antibody light chain or fragment thereof.
   38. The conjugate of any one of embodiments 31 to 36, wherein the knottin peptide is fused to the C-terminus of the antibody light chain or fragment thereof.
   39. The conjugate of any one of embodiments 31 to 38, wherein the drug is conjugated to the knottin peptide.
   40. The conjugate of any one of embodiments 31 to 38, wherein the drug is conjugated to the antibody light chain or fragment thereof.
   41. The conjugate of any one of embodiments 11 to 40, wherein the drug is a cytotoxic agent.
   42. The conjugate of any one of embodiments 11 to 40, wherein the drug is a toxin.
   43. The conjugate of any one of embodiments 11 to 40, wherein the drug is an anti-microtubule agent.
   44. The conjugate of embodiment 43, wherein the anti-microtubule agent is a tubulin inhibitor.
   45. The conjugate of embodiment 44, wherein the tubulin inhibitor is an auristatin.
   46. The conjugate of embodiment 45, wherein the tubulin inhibitor is an auristatin E or auristatin F.
   47. The conjugate of embodiment 46, wherein the tubulin inhibitor is monomethylauristatin E (MMAE) or monomethylauristatin F (MMAF).
   48. The conjugate of any one of embodiments 11 to 40, wherein the drug is a nucleoside drug.
   49. The conjugate of embodiment 48, wherein the nucleoside drug is a nucleoside analogue.
   50. The conjugate of embodiment 49, wherein the nucleoside analogue is selected from the group consisting of: gemcitabine, cytarabine, troxacitabine, decitabine, cladribine, fludarabine, clofarabine, and 2′-C-cyano-2′-deoxy-1-β-D-arabino-pentofuranosylcytosine (CNDAC).
   51. The conjugate of any one of embodiments 11 to 50, wherein the linker is an enzyme cleavable linker.
   52. The conjugate of embodiment 51, wherein the enzyme cleavable linker is a protease cleavable linker.
   53. The conjugate of embodiment 52, wherein the linker comprises a valine-citrulline dipeptide, a valine-alanine dipeptide, or both.
   54. The conjugate of embodiment 53, wherein the linker is a valine-citruline-paraaminobenzyloxy (Val-Cit-PAB) linker.
   55. The conjugate of embodiment 53, wherein the linker is a valylalanylparaaminobenzyloxy (Val-Ala-PAB) linker.
   56. The conjugate of any one of embodiments 1 to 55, wherein the knottin peptide is selected from the group consisting of: an EETI-II peptide, an AgRP peptide, a w-conotoxin peptide, a Kalata B1 peptide, an MCoTI-II peptide, an agatoxin peptide, and a chlorotoxin peptide.
   57. The conjugate of any one of embodiments 1 to 56, wherein the cell surface molecule is a cancer cell surface molecule.
   58. The conjugate of embodiment 57, wherein the cancer cell surface molecule is present on cancer cells of a solid tumor.
   59. The conjugate of any one of embodiments 1 to 57, wherein the cell surface molecule is a cell surface receptor.
   60. The conjugate of embodiment 59, wherein the cell surface receptor is a cell adhesion receptor.
   61. The conjugate of embodiment 60, wherein the cell adhesion receptor is an integrin.
   62. The conjugate of embodiment 60, wherein the integrin is selected from the group consisting of: αvβ1 integrin, αvβ3 integrin, αvβ5 integrin, αvβ6 integrin, α5β1 integrin, and any combination thereof.
   63. The conjugate of embodiment 59, wherein the cell surface receptor is a chemokine receptor.
   64. The conjugate of embodiment 63, wherein the chemokine receptor is C—X—C chemokine receptor type 4 (CXCR4).
   65. The conjugate of embodiment 59, wherein the cell surface receptor is a growth factor receptor.
   66. The conjugate of embodiment 59, wherein the cell surface receptor is an immune cell receptor.
   67. The conjugate of embodiment 66, wherein the immune cell receptor is cytotoxic T-lymphocyte-associated protein 4 (CTLA-4).
   68. The conjugate of embodiment 59, wherein the cell surface receptor is neuropilin-1 (NRP1).
   69. The conjugate of any one of embodiments 1 to 57, wherein the cell surface molecule is a membrane protease.
   70. The conjugate of embodiment 69, wherein the membrane protease is matriptase.
   71. A composition comprising the conjugate of any one of embodiments 1 to 70.
   72. The composition of embodiment 71, wherein the composition is a pharmaceutical composition comprising:
the conjugate; and
a pharmaceutically acceptable carrier.
   73. The composition of embodiment 72, wherein the pharmaceutical composition is formulated for parenteral administration.
   74. The composition of embodiment 72, wherein the pharmaceutical composition is formulated for oral administration.
   75. A kit comprising:
a therapeutically effective amount of the pharmaceutical composition of embodiment 72; and
instructions for administering the pharmaceutical composition to an individual in need thereof.
   76. The kit of embodiment 75, wherein the pharmaceutical composition is present in one or more unit dosages.
   77. A method comprising administering a therapeutically effective amount of the pharmaceutical composition of embodiment 72 to an individual in need thereof.
   78. The method according to embodiment 77, wherein the individual has cancer and the engineered loop binds to a cell surface molecule on cancer cells present in the individual.
   79. The method according to embodiment 78, wherein the cell surface molecule is a cell surface receptor.
   80. The method according to embodiment 79, wherein the cell surface receptor is a cell adhesion receptor.
   81. The method according to embodiment 80, wherein the cell adhesion receptor is an integrin.
   82. The method according to embodiment 81, wherein the integrin is selected from the group consisting of: αvβ1 integrin, αvβ3 integrin, αvβ5 integrin, αvβ6 integrin, α5β1 integrin, and any combination thereof.
   83. The method according to embodiment 82, wherein the individual has a solid tumor comprising the cancer cells.
   84. The method according to embodiment 83, wherein the solid tumor is a brain tumor.

The following examples are offered by way of illustration and not by way of limitation.

Experimental

### Example 1—Production of Knottin-Based Conjugates

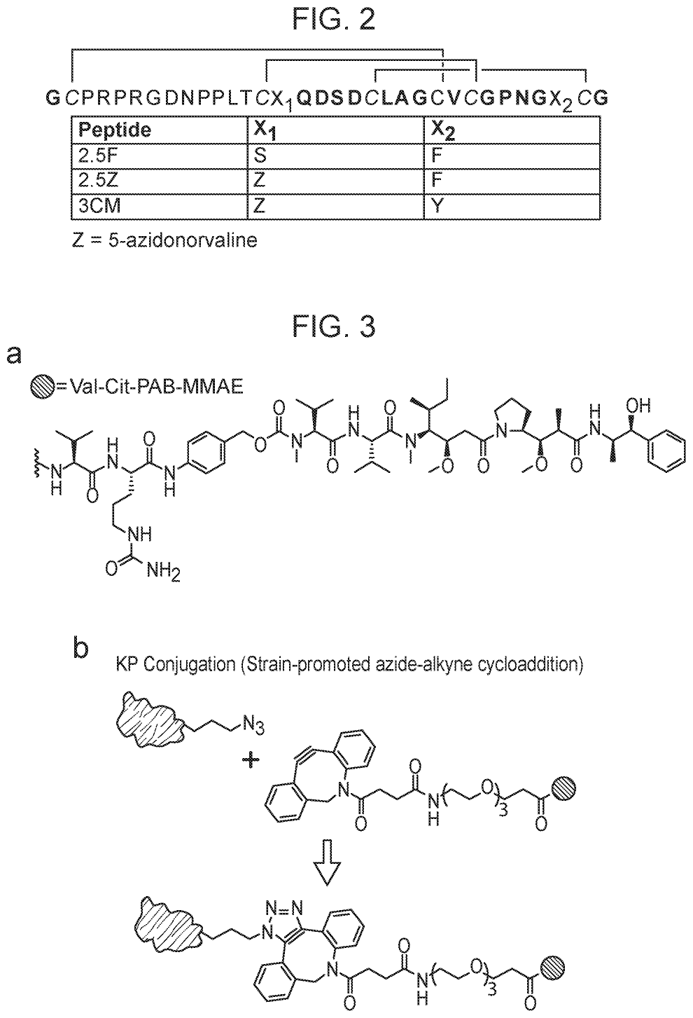

To create a set of protein-drug conjugates spanning the size range of protein biologics, a knottin (K), knottin-Fc fusion (KFc), and knottin-Ab fusion (KAb) were produced (FIG. 1B). The knottin was based on the previously engineered knottin, EETI 2.5F, and modified for ease of concentration measurement (FIG. 2). The knottin was produced by solid-phase peptide synthesis then folded and purified. EETI 2.5F was then genetically fused to a human IgG1 Fc domain to create KFc and genetically fused to the N-terminus of a full-length human IgG1 antibody against an irrelevant binding target (anti-FITC or anti-human CEA) to create KAb.

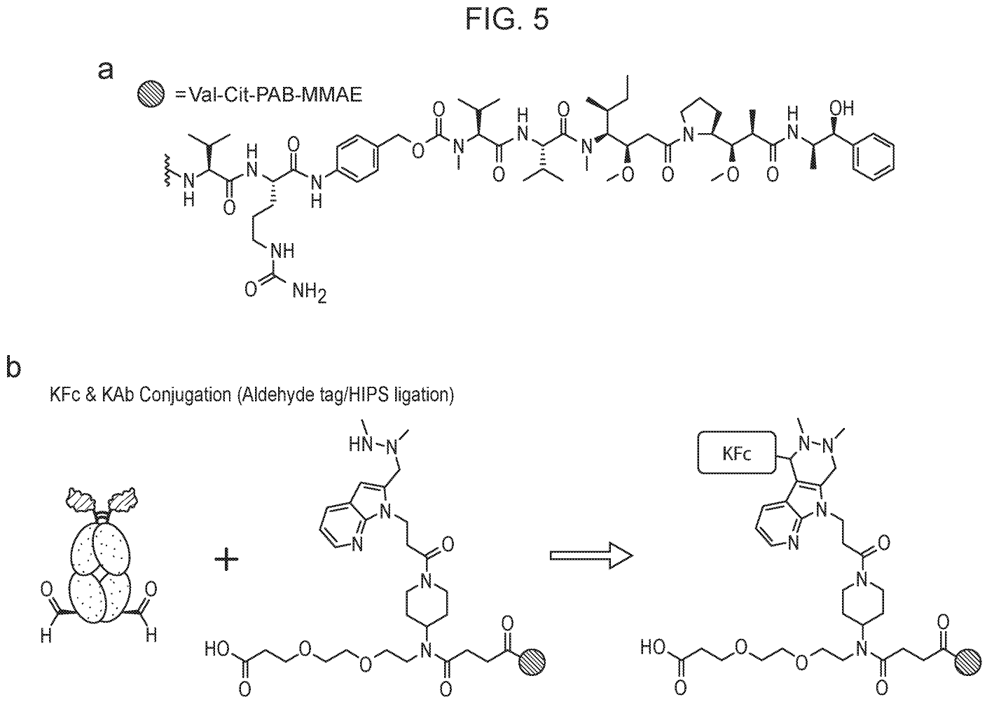

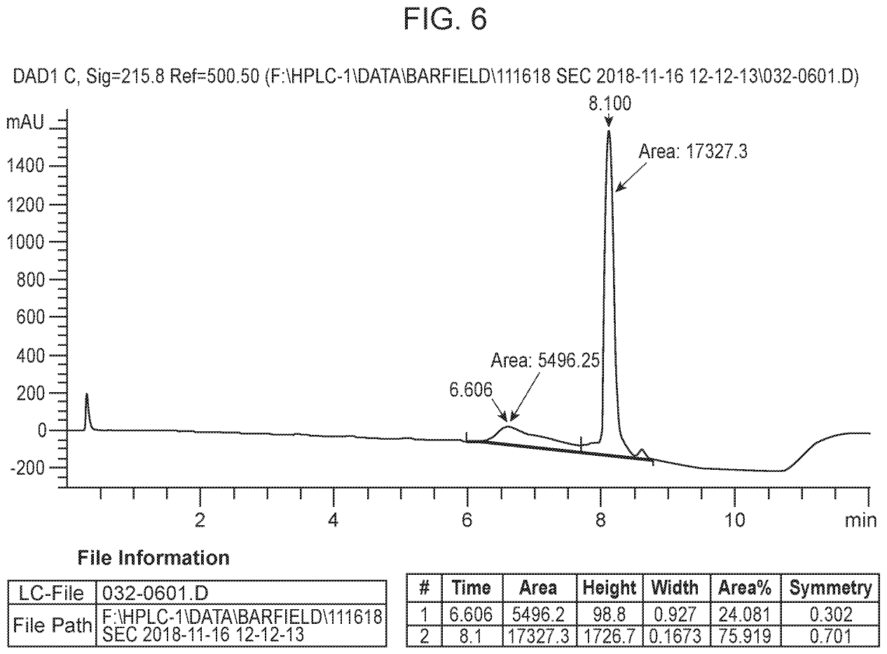

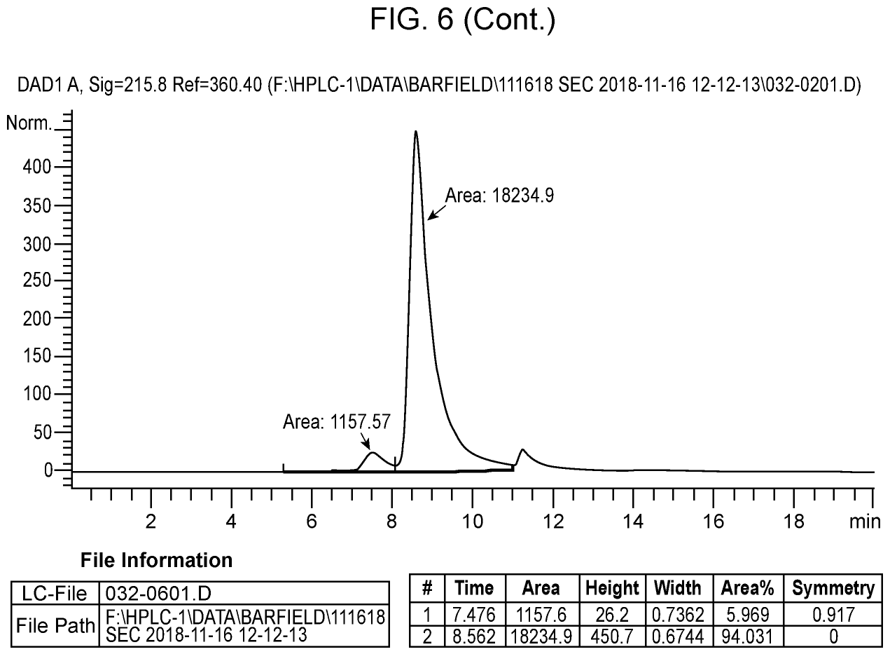

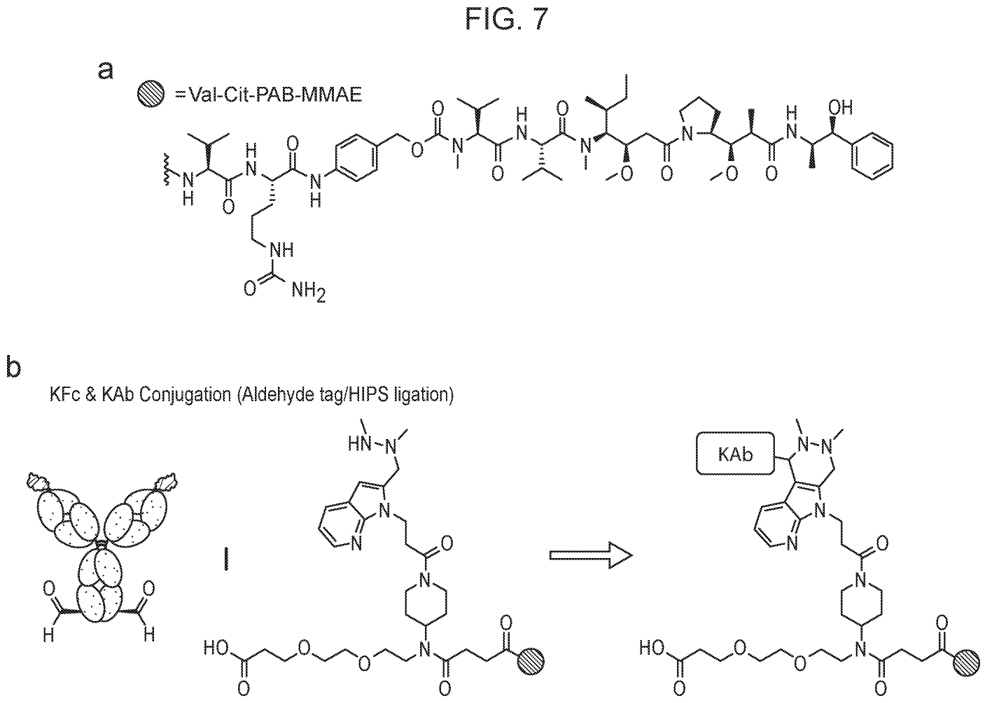

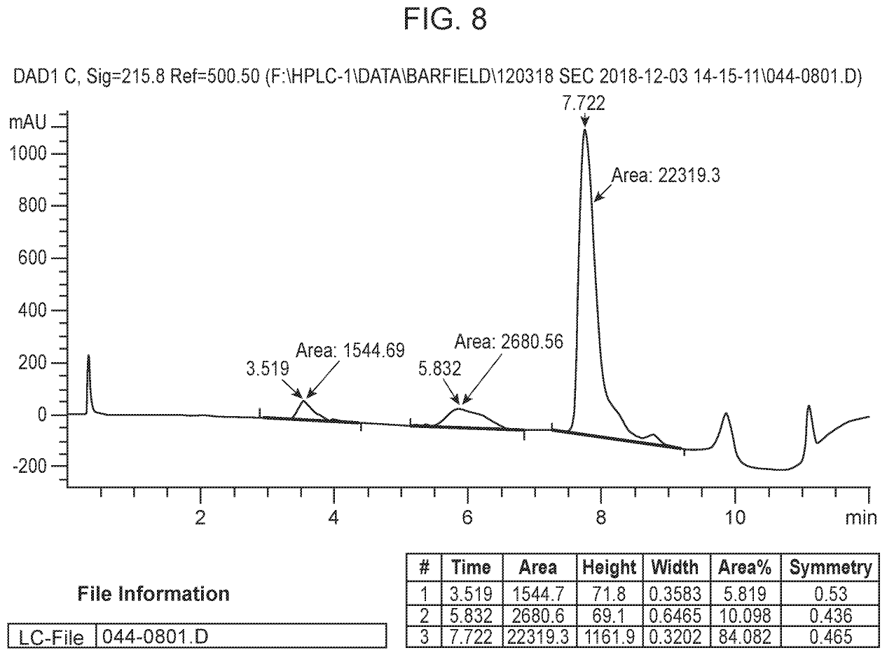

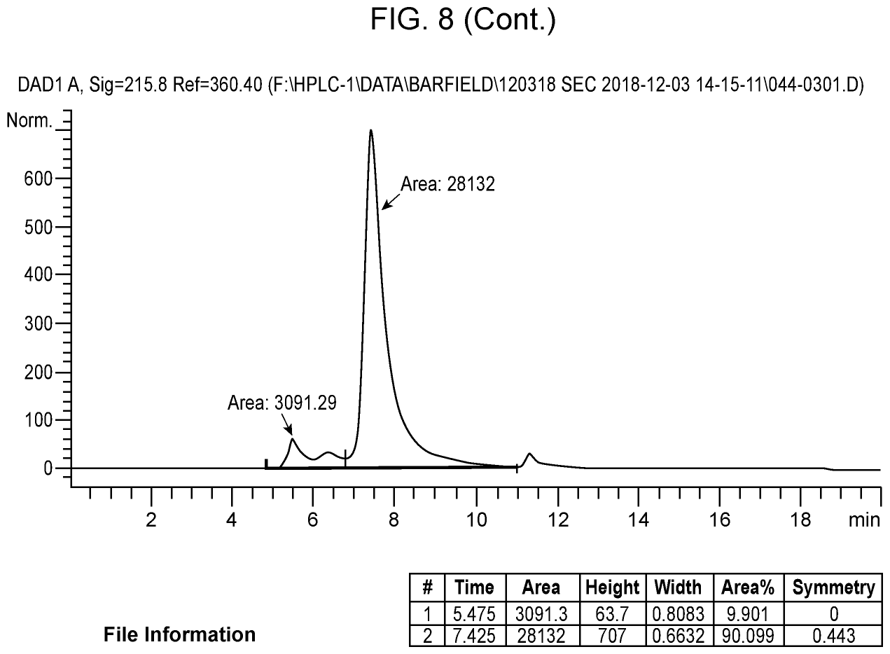

The knottin-drug conjugate (KDC) was prepared by reacting K with a Val-Cit-PAB MMAE derivative containing a DBCO linker via copper-free, strain-promoted cycloaddition (FIG. 3, 4). The knottin-Fc-drug conjugate (KFDC) and knottin-antibody-drug conjugate (KADC) were prepared by Hydrazino-Pictet-Spengler ligation⁸⁹ of aldehyde tagged KFc or KAb with a HIPS linker MMAE derivative (FIG. 5-8).

### Example 2—In Vitro Characterization

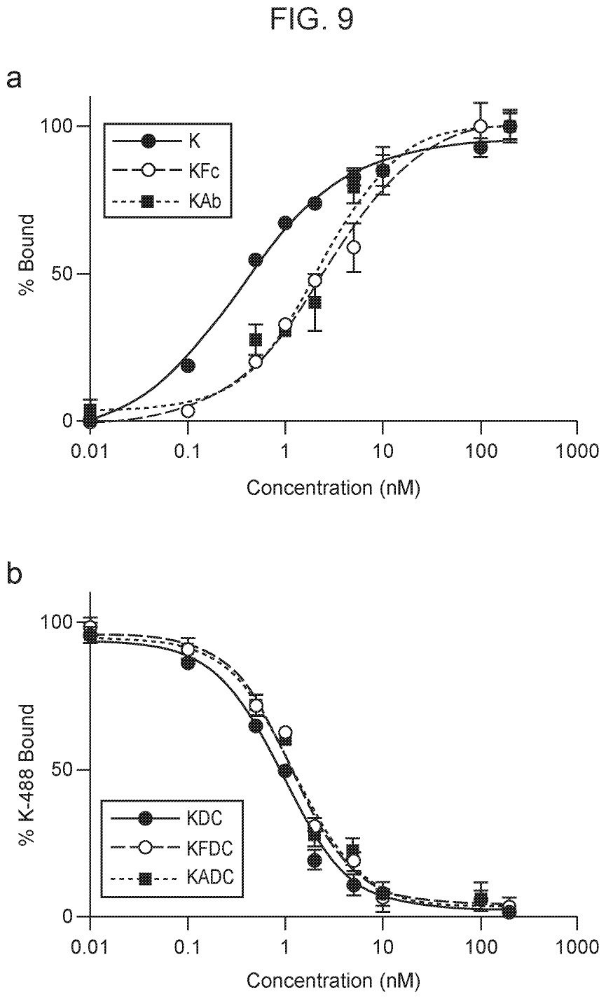

The binding affinity of the constructs described in Example 1 were measured using U87MG glioblastoma cells. Drug-free protein affinities were measured directly using Alexa Fluor 488-conjugaed proteins and drug conjugates were measured via a competition binding assay displacing Alexa Fluor 488-labeled K. All constructs tested bound to U87MG cells with low-nanomolar affinity (FIG. 9A-B). The measured affinities were consistent with previous studies, indicating that neither genetic fusion to the Fc or Ab, nor the conjugation to MMAE appreciably interfere with tumor targeting by the knottin.

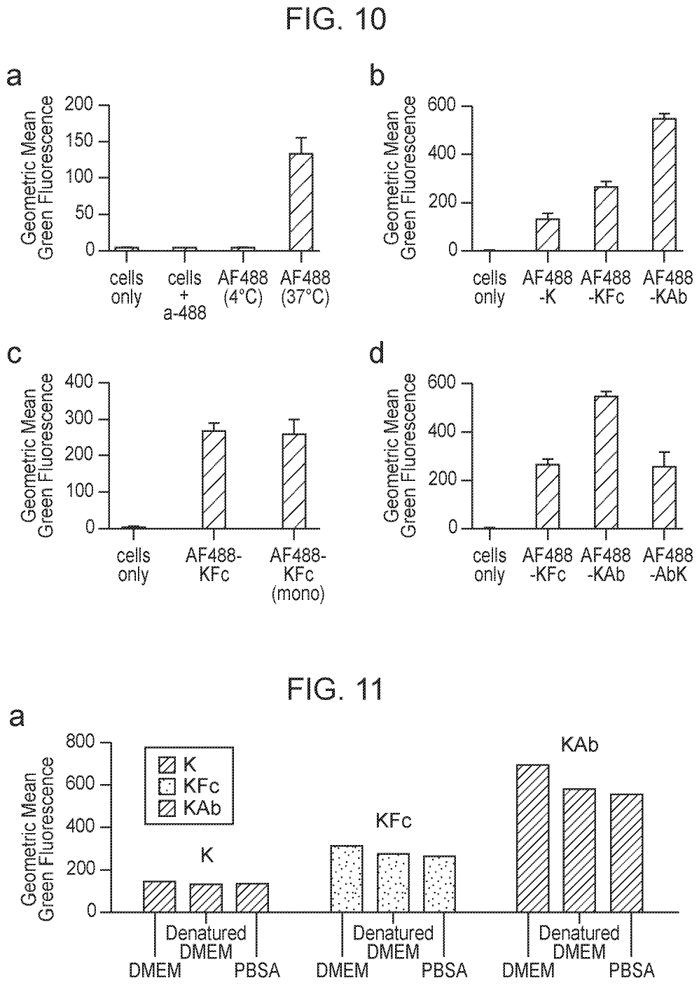

The cleavage of the Val-Cit-PAB linker and the subsequent release and mechanism of action of MMAE are dependent upon intracellular delivery. Thus, it was important to validate that each construct internalized efficiently. Internalization was measured by incubating Alexa Fluor 488-conjugated proteins with cells at 37° C. After a 4 h incubation, surface-bound protein was quenched using an anti-AF488 antibody, that binds any exposed fluorophores. As shown in FIG. 10A, if internalization is inhibited by incubating cells at 4° C. to freeze the plasma membrane, measured fluorescence returns to baseline.

All constructs showed substantial internalization above controls (FIG. 10B). The relative internalization of each compound was, from least to greatest, K, KFc, then KAb. To determine if bivalency was primarily responsible for the increase in internalization of KFc over K, a monovalent, 1-armed KFc was tested. This monovalent Fc showed similar internalization (FIG. 10C), indicating valency is not the primary driver of increased internalization. The constrained Fc hinge region likely limits the ability of KFc to fully exploit divalent interactions or cluster multiple integrin pairs. KAb, conversely, has far greater flexibility between knottins. An antibody-knottin (AbK) construct, with EETI 2.5F genetically fused to the C-terminus rather than the N-terminus, was constructed to test if limiting the inter-knottin distance and flexibility would decrease internalization. As shown in FIG. 10D, AbK had a similar level of internalization to KFc, suggesting that KAb is more easily able to bind to and cluster multiple integrin pairs.

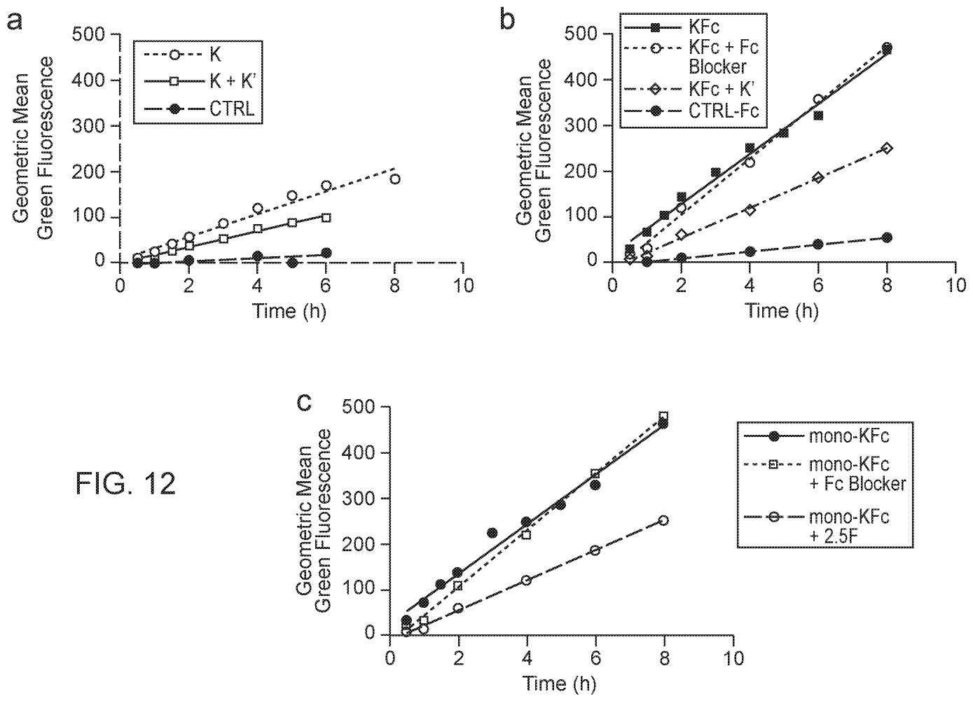

Because both KFc and AbK internalized more readily than K, it was hypothesized that the Fc could be promoting internalization via Fc receptor or complement-mediated pathways. Internalization of K, KFc, and KAb were tested in heat-denatured media and a buffer of PBS and 0.1% bovine serum albumin (FIG. 11). None of the constructs incubated in heat-denatured media or PBS had significantly reduced internalization compared to those incubated in complete media, suggesting that active proteins within the media (c1q, etc.) do not appreciably contribute to the increased internalization of KFc and KAb. The rate of internalization was then measured for each K, KFc, monovalent KFc, and KAb and compared to when these constructs were incubated with cells in the presence of an Fc-blocking protein or excess unconjugated K (FIG. 12).

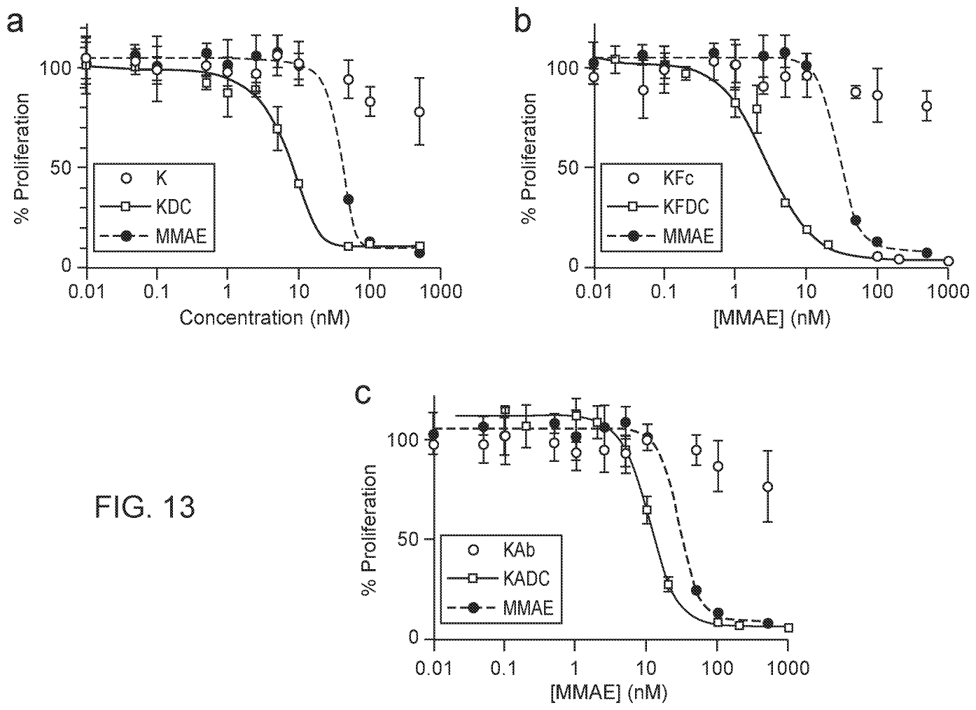

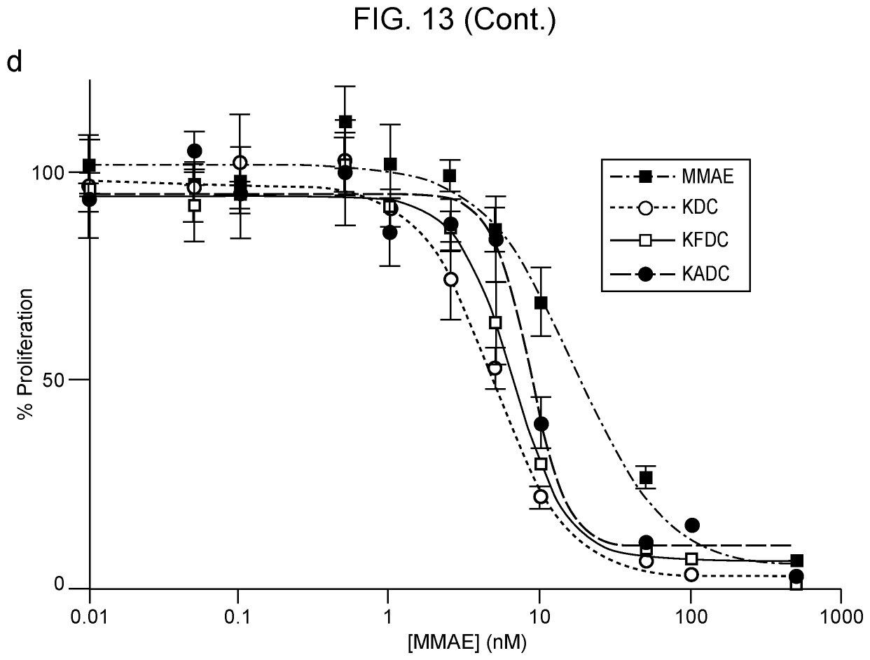

The in vitro efficacy of each drug conjugate was tested by measuring the inhibition of U87MG cell growth (FIG. 13). Unconjugated proteins were well-tolerated and did not induce significant cell death when dosed up to 100 nM (FIG. 13A-C). MMAE alone showed moderate efficacy with an ED₅₀ of 16.8 nM. KDC, KFDC, and KADC all demonstrated slightly increased potency in vitro with respective ED₅₀ values of 4.9, 6.5, and 8.9 nM (FIG. 13D).

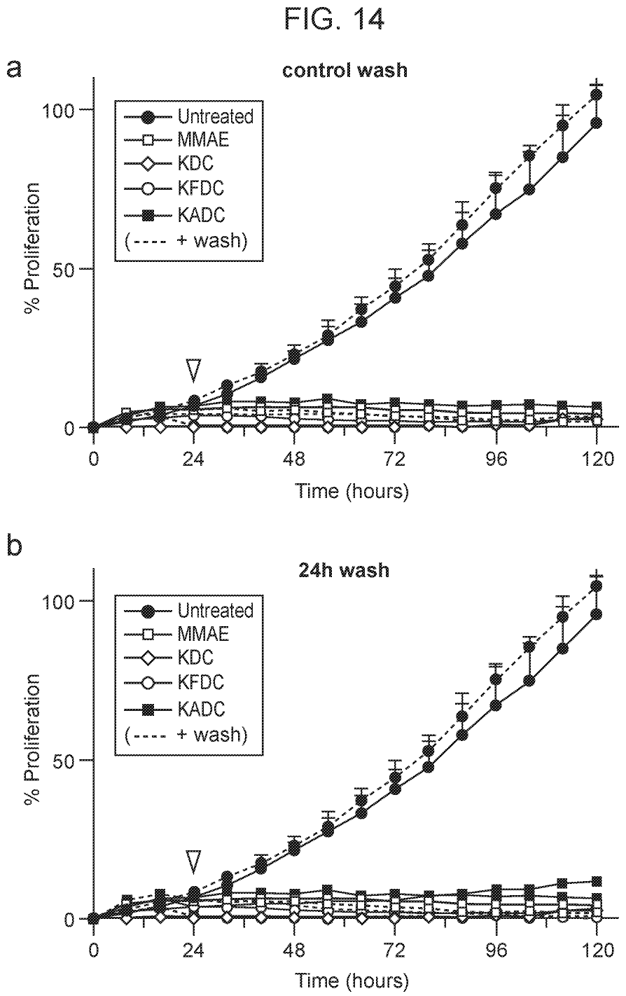

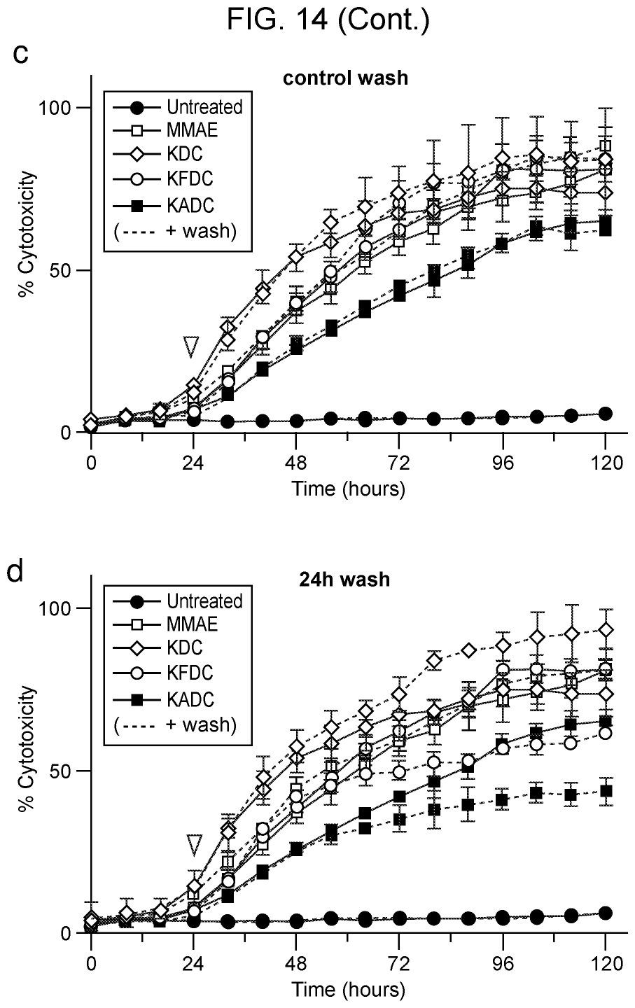

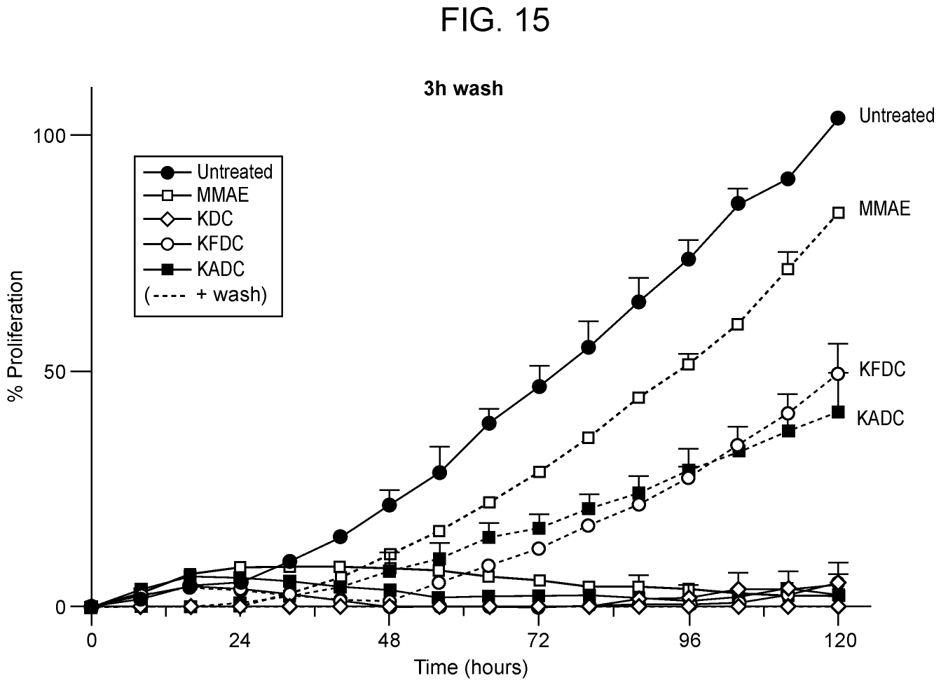

To assess the activity of each drug conjugate in a model more representative of in vivo therapy, a similar assay was set up incubating RFP-expressing U87MG cells with SYTOX green and imaging every 4 h. After several time points, cells were washed and media was replaced with either fresh, drug-containing media or fresh, drug-free media (FIG. 14). All conjugates maintained high potency and inhibited proliferation after only 24 h exposure (FIG. 14B). The ability of each drug conjugate to inhibit proliferation was further investigated to reveal that KDC continued to demonstrate complete cell inhibition only 3 h of exposure (FIG. 14C). The inhibition of cellular proliferation mirrored the measured cytotoxicity. All conjugates induced nearly 100% cell death when incubated without washing, with a wash of drug-containing media, or with washing after 24 h (FIG. 14D, 14E). Each construct was also evaluated under an even more stringent condition, with washing after 3 h of exposure. KDC maintained potent activity and resulted in >95% inhibition of cell proliferation (FIG. 15). KFDC and KADC experienced substantial reductions in their respective potencies and each inhibited ˜50% of cell proliferation.

### Example 3—Pharmacokinetics

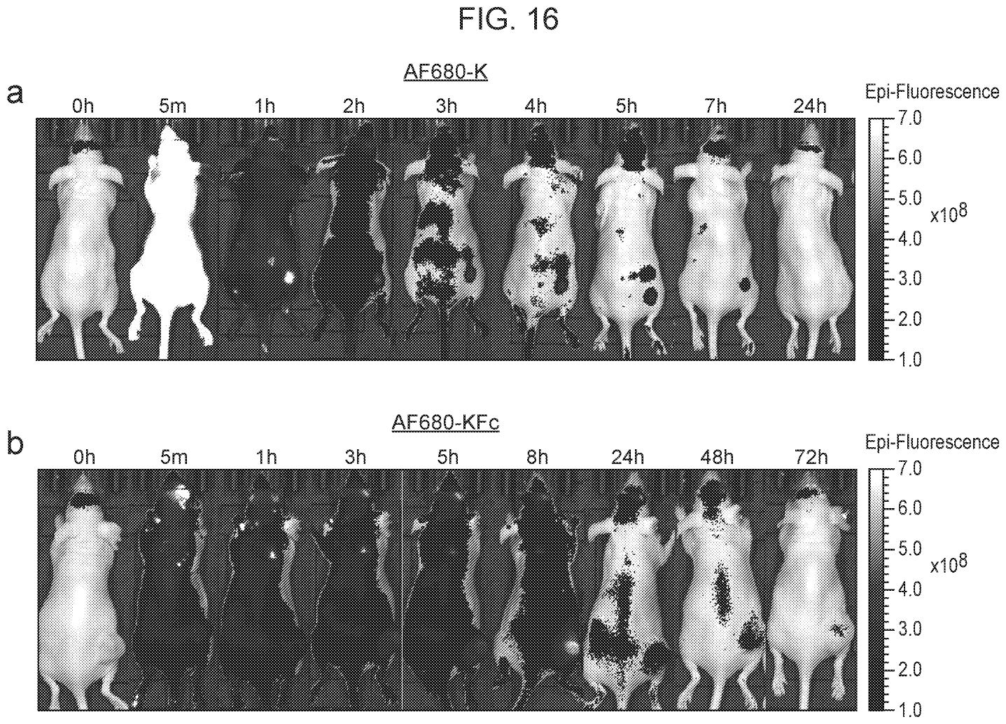

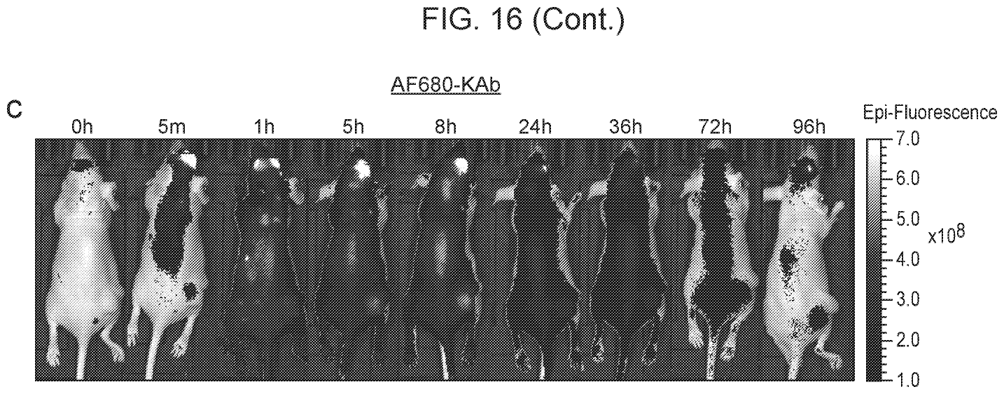

The ability of each drug conjugate to target tumors in vivo was evaluated by intravenously injecting Alexa Fluor 680-conjugated proteins to mice bearing U87MG glioblastoma hip xenografts (FIG. 16). Fluorescence was monitored by non-invasive, near-infrared fluorescence imaging using an IVIS Spectrum. At each time point, the radiant efficiency of a region of interest over the tumor and another control ROI were calculated to approximate the beta half-life of fluorescently labeled drug conjugates in the blood and compare that to retained tumor signal.

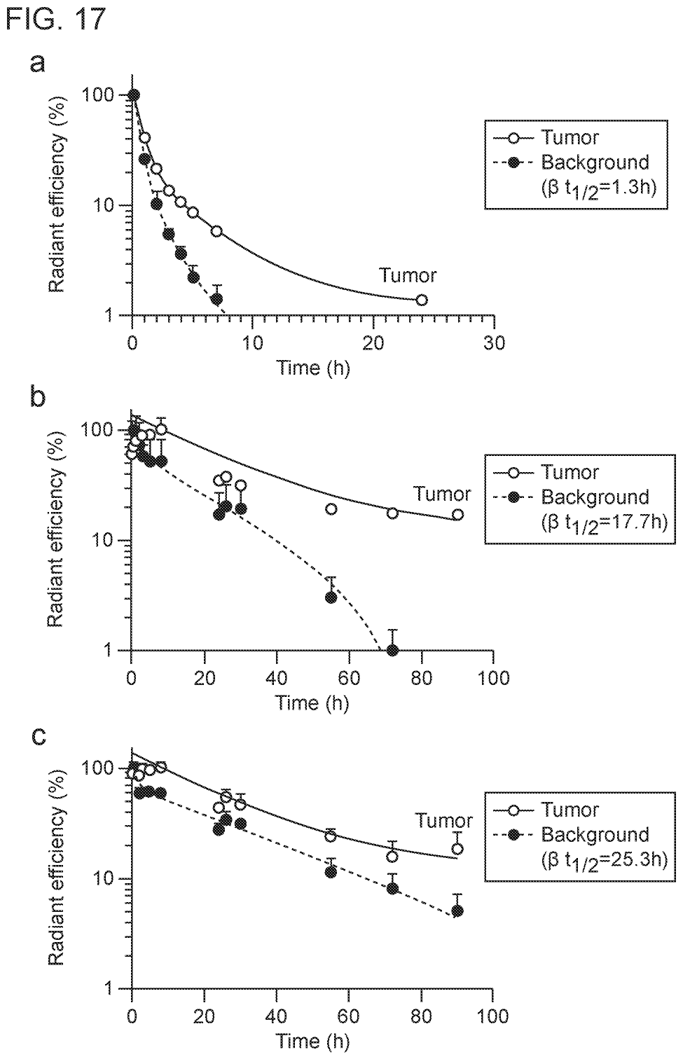

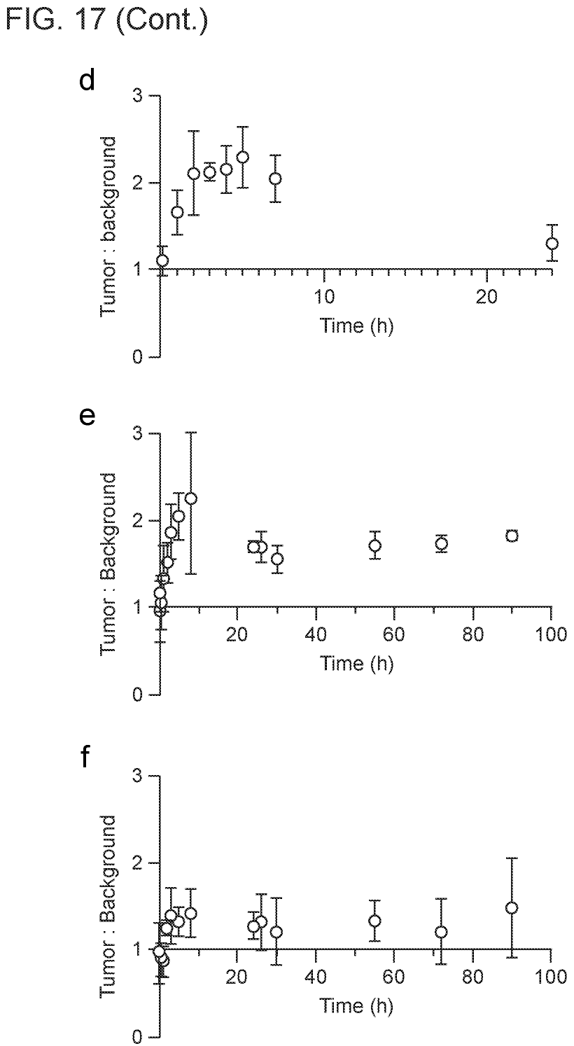

The pharmacokinetic profile of tumor-targeting vehicles is determined in part by size. Low MW vehicles below the size cutoff for glomerular filtration (generally reported in the literature to be ˜70 kDa) are more likely to be quickly eliminated via kidney filtration. The KDC (˜5 kDa) is well below the renal clearance cutoff and had a beta half-life of 1.4 h, suggesting rapid kidney clearance (FIG. 17A). The KFDC (˜65 kDa) is close in MW to the renal clearance cutoff and had a beta half-life of 17.7 h, potentially extended additionally through the Fc-mediated half-life extension (FIG. 17B). The KADC (˜150 kDa) had the longest measured beta half-life of 25.3 h (FIG. 17C). In each conjugate, tumor signal was substantially higher than background and persisted after background signal returned to baseline. For KDC, the maximum tumor-to-background ratio was observed 3-5 h post administration (FIG. 17D). KFDC obtained a maximum tumor-to-background ratio approximately 10 h post administration and maintained a high ratio for several days (FIG. 17E). KADC maintained a moderate tumor-to-background ratio that persisted and even increased for over a week (FIG. 17F).

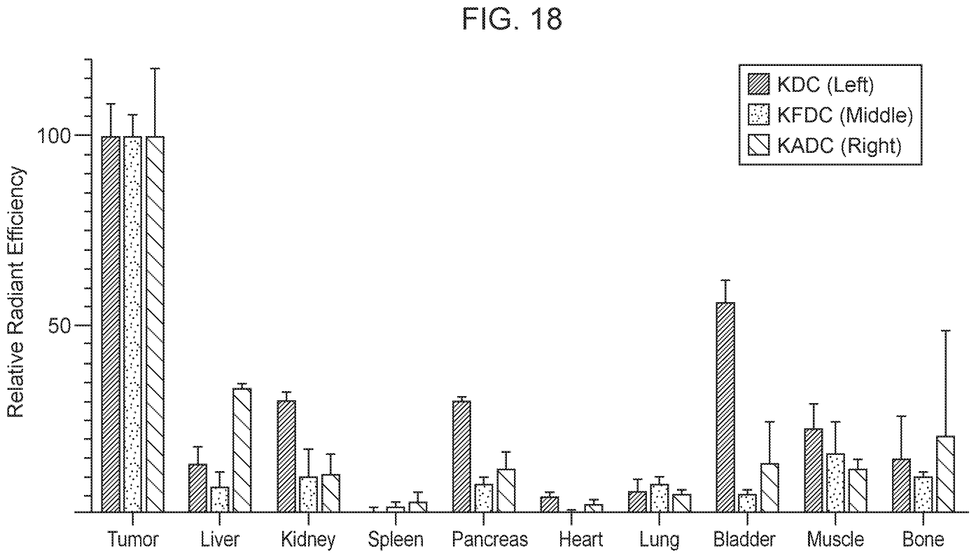

To further assess the tumor-to-background ratio, the uptake of fluorescently-labeled drug conjugate by each organ was measured ex vivo. Nude mice bearing U87MG glioblastoma hip xenografts were injected intravenously with Alexa Fluor 680-conjugated proteins. Mice were sacrificed after 3 or 24 h and organs were removed for imaging. The radiant efficiency of excised tumors was significantly higher than all other measured organs. Specifically, the liver, kidneys, spleen, pancreases, heart, lungs, bladder, quadriceps, and femur (FIG. 18). Notably, these ex vivo data corroborate the hypothesis that KDC is filtered by the kidneys while KADC are metabolized in the liver and KFDC is somewhere in between these two. KADC shows substantially higher liver uptake than KFDC or KDC, though still <30% of the signal in the tumor. KDC had higher kidney and bladder uptake, indicative of clearance through the renal filtration and elimination in the urine.

### Example 4—In Vivo Efficacy

The high affinity binding, rapid internalization and potent inhibition of U87MG proliferation in vitro suggested that each drug conjugate has significant potential in a murine model. Imaging and pharmacokinetic data confirm that KDCs have a short circulatory half-life while KFDCs and especially KADCs persist in the blood for several days. Thus, in vivo therapeutic efficacy studies were set up to evaluate the performance of each drug conjugate.

MMAF-conjugated KFDC had moderate efficacy when dosed 3 times per week at 5 mg/kg (data not shown). Using this treatment regimen as a starting point, a preliminary study was set up using 5 mice per treatment group. Mice were inoculated with 5×10⁶ U87MG cells in the right flank and tumors were allowed to grow and establish until reaching an approximate area of 30 mm². Mice were then randomized into 1 of 4 treatment groups: PBS, KDC, KFDC, or KADC. To keep groups equivalent and provide the highest chance of efficacy for KDC despite its short circulatory half-life, all drug conjugate groups dosed 3 times per week for 3 weeks. Each dose contained the appropriate drug conjugate normalized to 2.38 nmol MMAE (˜0.6 mg/kg KDC, ˜5 mg/kg KFDC, ˜10 mg/kg KADC).

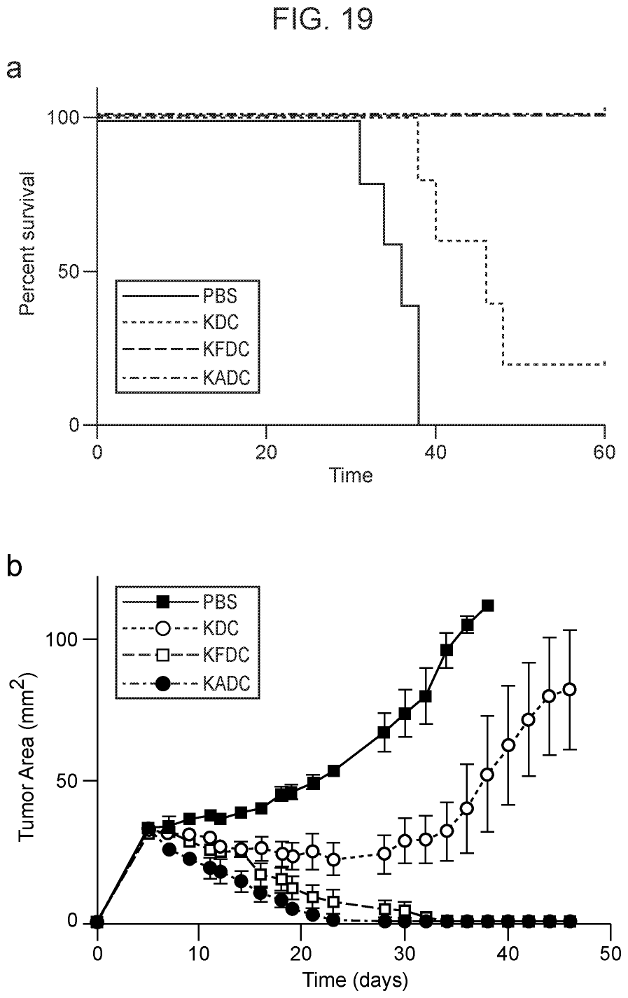

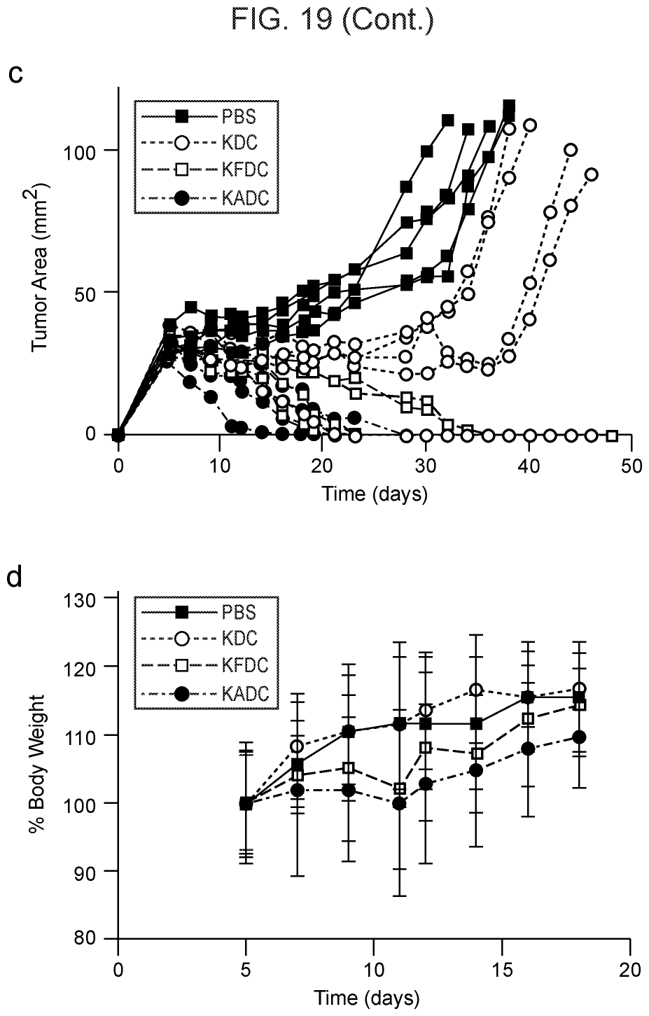

All drug conjugates significantly extended survival over control mice (FIG. 19A, 19B). KDC induced complete tumor regression in 1 of 5 mice and maintained a cytostatic effect in the remaining 4 of 5 mice (FIG. 19C). After 3 weeks of treatment, the tumors of these 4 mice returned. Both KFDC and KADC resulted in complete tumor regression in all 5 mice per respective treatment group (FIG. 19C). The weight of all mice was also recorded for the duration of the experiment to monitor for signs of severe toxicity. The weights of all groups were indistinguishable, suggesting that compounds were well-tolerated (FIG. 19D).

The data in FIG. 19 demonstrate that all drug conjugates are capable of therapeutic effect. To further investigate each compound, several changes were made to the experimental design. Because of the KDC's rapid systemic clearance, a significant portion of the injected dose is likely lost and the ˜0.6 mg/kg dose was therefore increased substantially. Because dosages for chemotherapeutics are conventionally determined as mass of chemotherapeutic per patient body surface area, a more clinically relevant comparison of equal mass was selected over an equimolar comparison. Next, a once-per-week dosing regimen was selected to coincide with realistic clinical dosing considerations. Finally, tumors were implanted with Matrigel matrix to increase tumor growth and with only 2.5×10⁶ cells per injection to decrease initial tumor necrosis. Mice bearing U87MG hip xenografts were equally distributed into the following treatment groups with intravenous injections given once weekly for 4 weeks: KDC, KFDC, KADC, unconjugated proteins, or a PBS control. Each drug conjugate was also evaluated at a low dose and a high dose.

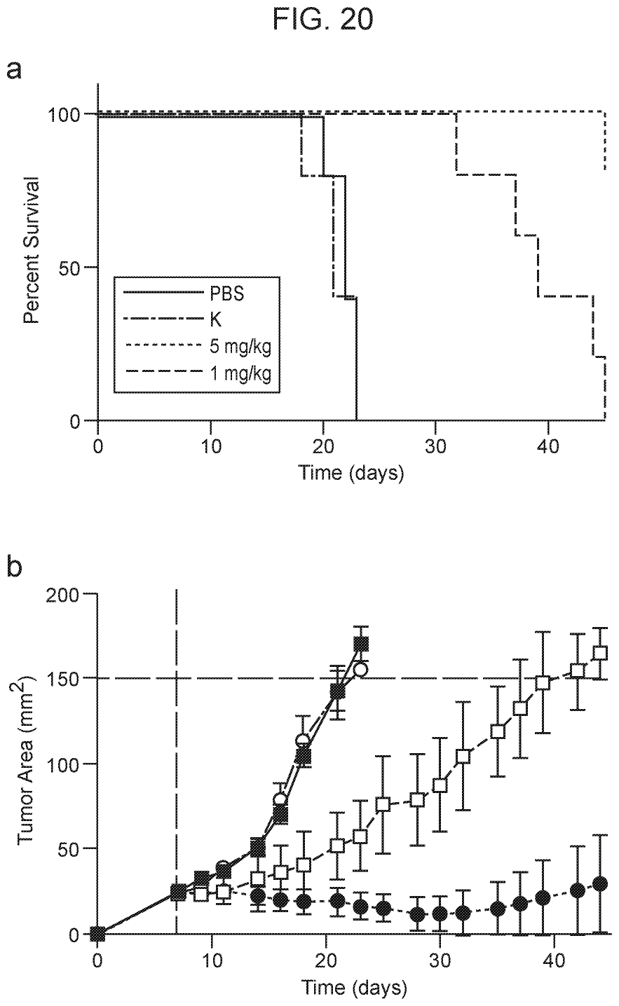

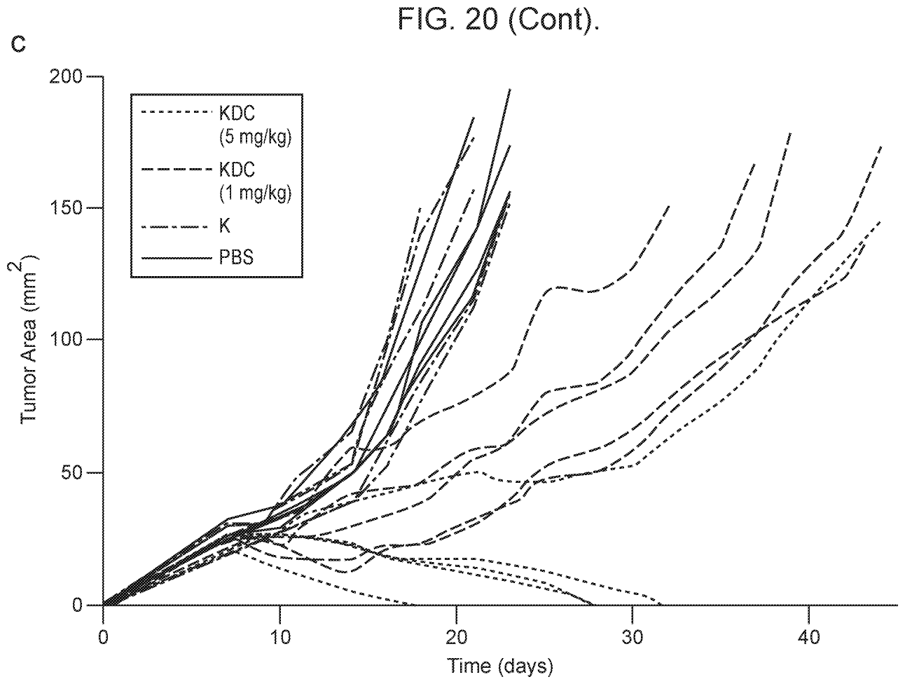

The low dose of KDC (1 mg/kg) performed similarly to the pilot experiment, despite the optimization to allow more aggressive tumor growth (FIG. 20A, 20B). KDC dosed 1 mg/kg significantly delayed tumor growth but did not cause tumor regression and tumors regrew in 5 of 5 mice after the end of treatment (FIG. 20C). The high dose of KDC (5 mg/kg) was very effective and caused complete regression of tumors in 4 of 5 mice and significantly delayed tumor growth in the remaining mouse (FIG. 20C). Mice treated with unconjugated K were indistinguishable from PBS control mice.

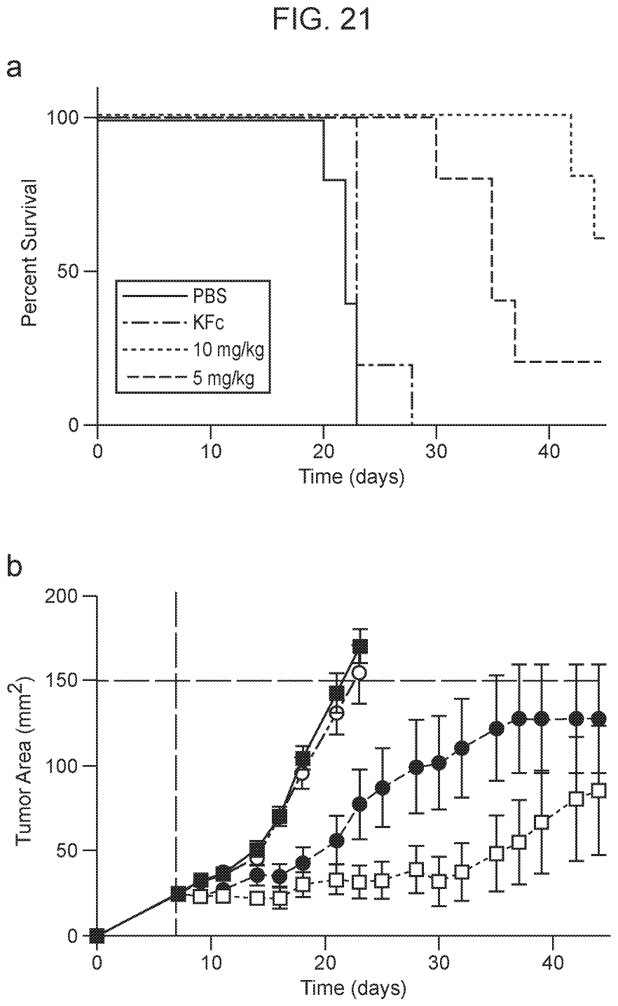

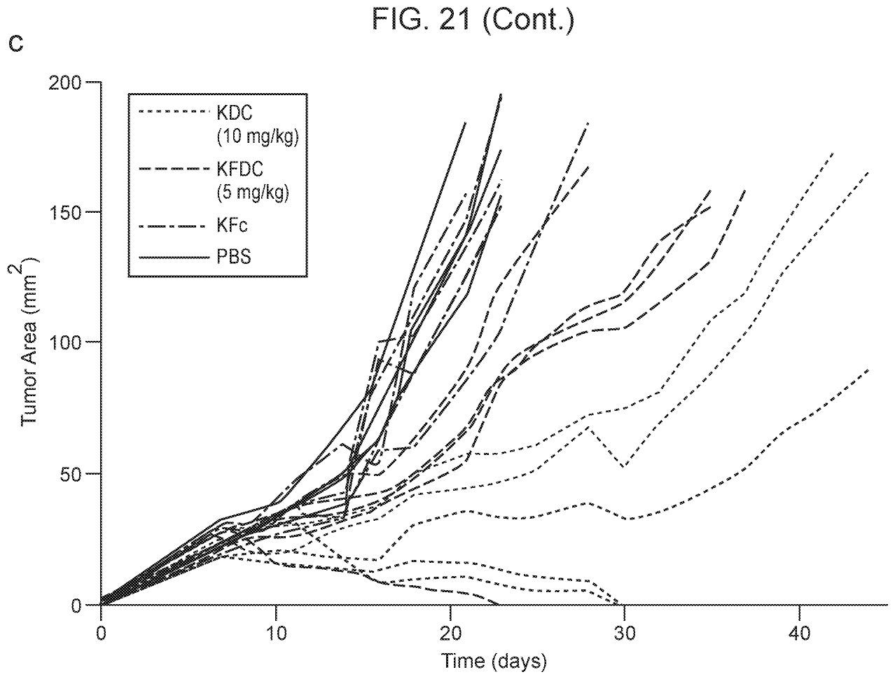

In contrast to the pilot experiment, KFDC and KADC were substantially reduced in efficacy in the more aggressive tumor model. The low dose of KFDC (5 mg/kg) moderately inhibited tumor growth (FIG. 21A, 21B) and caused tumor regression in 1 of 5 mice. Tumor growth was slowed, but not stopped, in the remaining 4 of 5 mice (FIG. 21C). The high dose of KFDC (10 mg/kg) caused complete regression of tumors in 2 of 5 mice and significantly delayed tumor growth in the remaining 3 of 5 mice (FIG. 21C). Mice treated with unconjugated KFc were indistinguishable from PBS control mice.

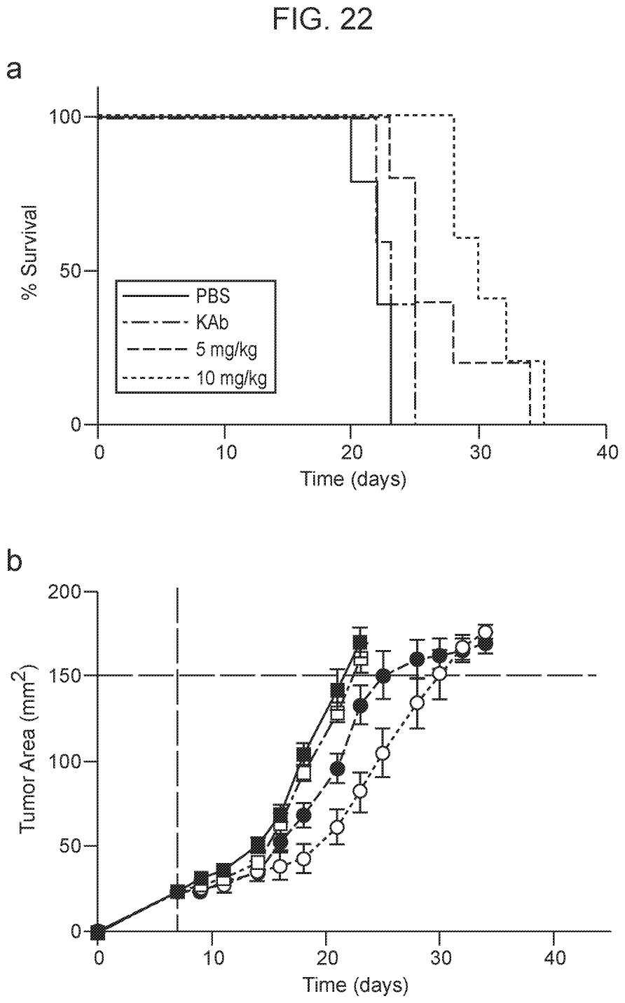

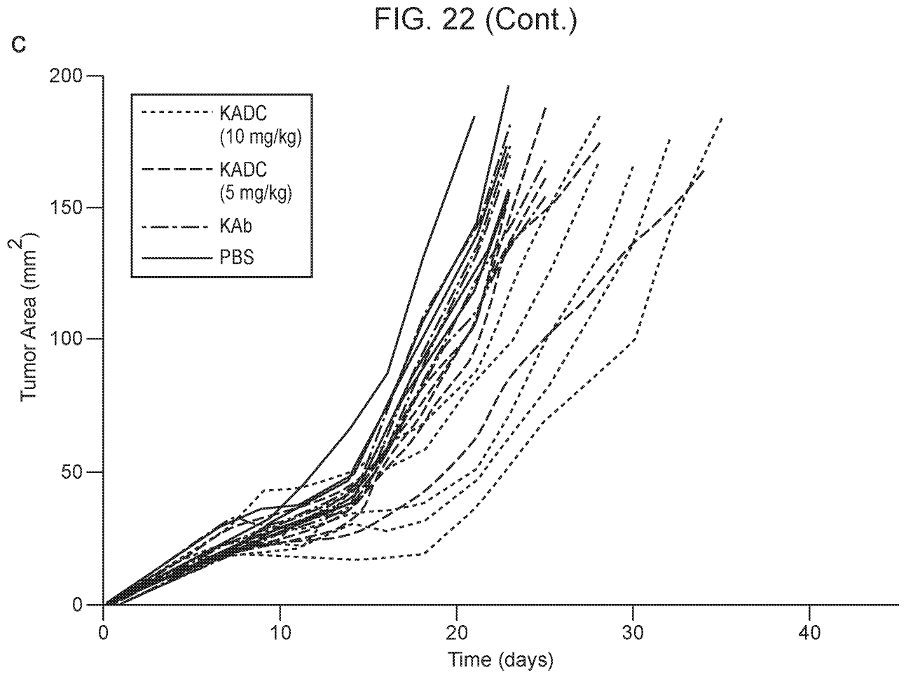

The low dose of KADC (5 mg/kg) was minimally effective (FIG. 22A, 22B). Tumor growth was only marginally slowed in 5 of 5 mice (FIG. 22C). The high dose of KADC (10 mg/kg) also lacked significant efficacy (FIG. 22A, 22B) and moderately slowed tumor growth in all mice but did not induce any tumor regression (FIG. 22C). Mice treated with unconjugated KAb (anti-CEA) were indistinguishable from PBS control mice.

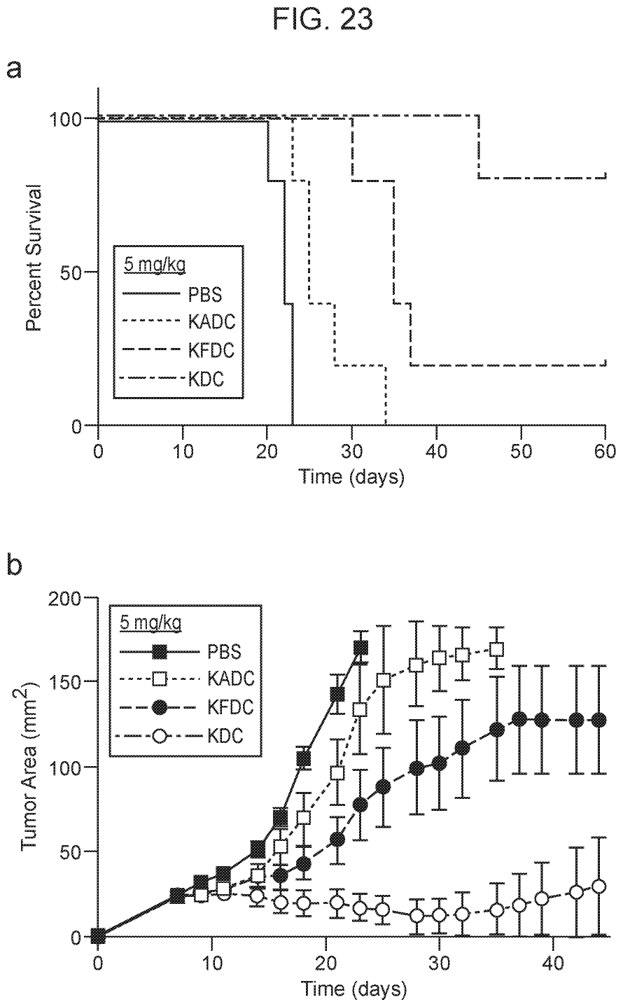

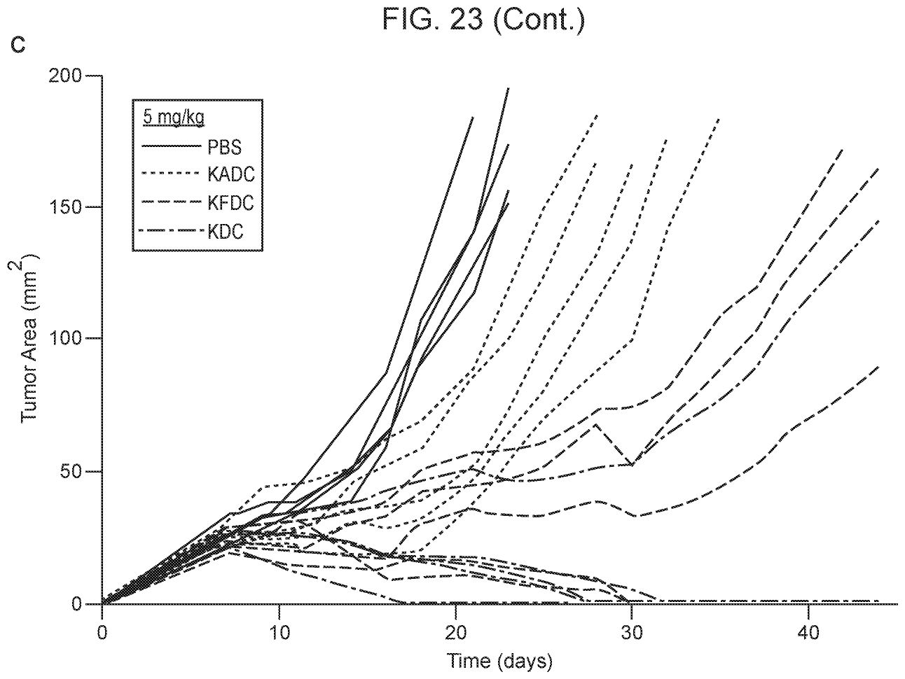

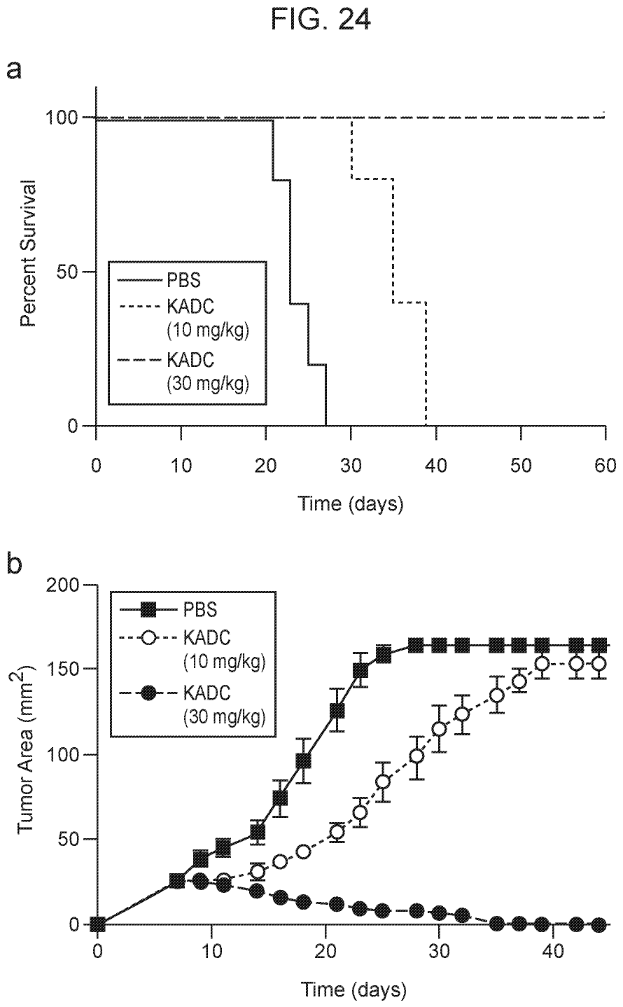

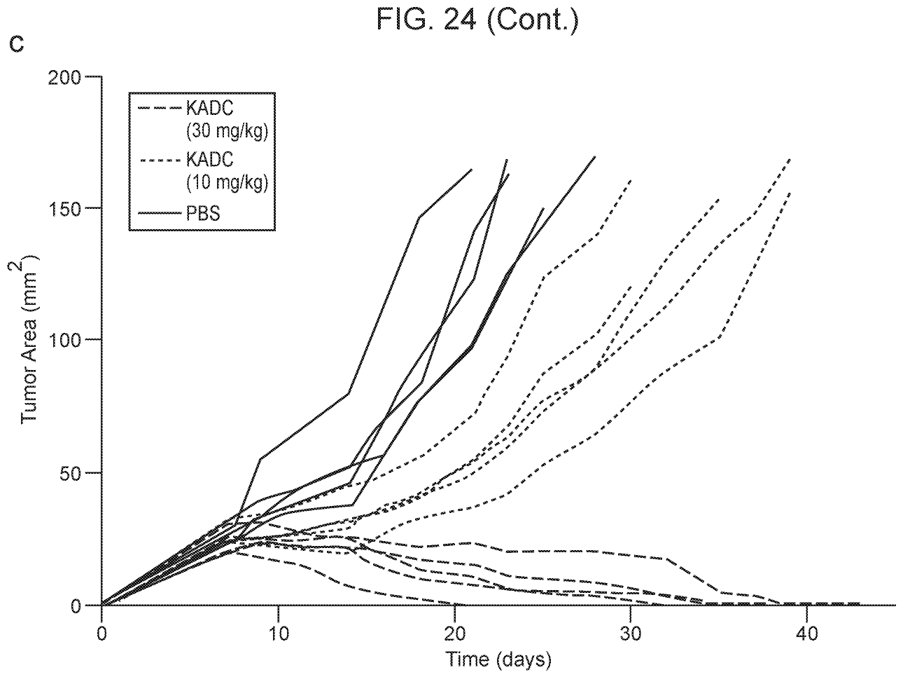

When comparing drug conjugates using the clinically relevant comparison of mass of drug conjugate per kg mouse body weight, KDC significantly outperformed both KFDC and KADC (FIG. 23). To understand the discrepancy between these results and the pilot study, mice were also treated with KADC administered at a much higher dose of 30 mg/kg, closer to an equimolar dose of MMAE. In this experiment, KADC was very effective and induced complete tumor regression in 5 of 5 mice (FIG. 24).

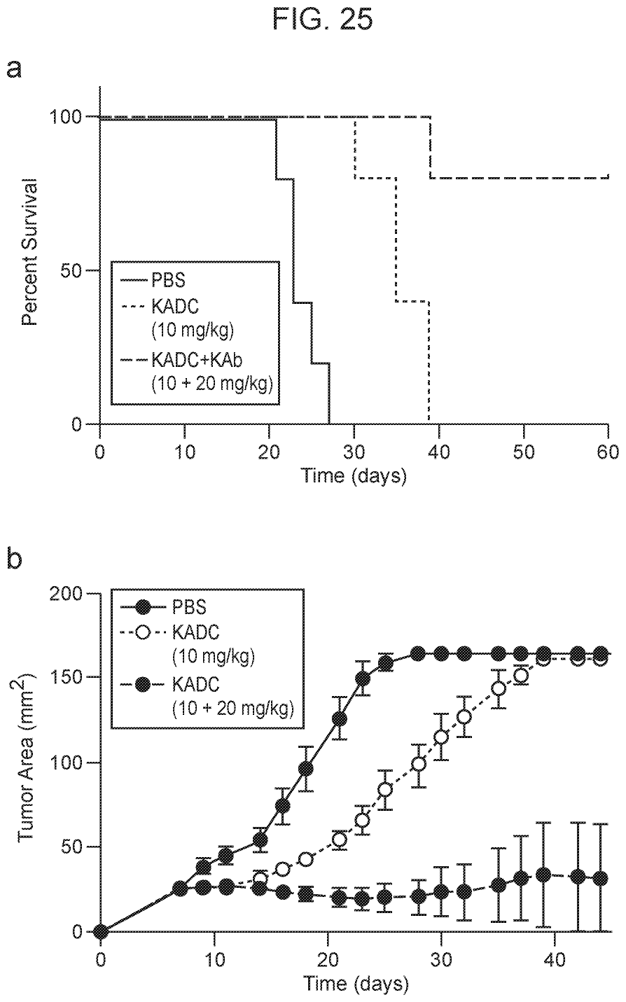

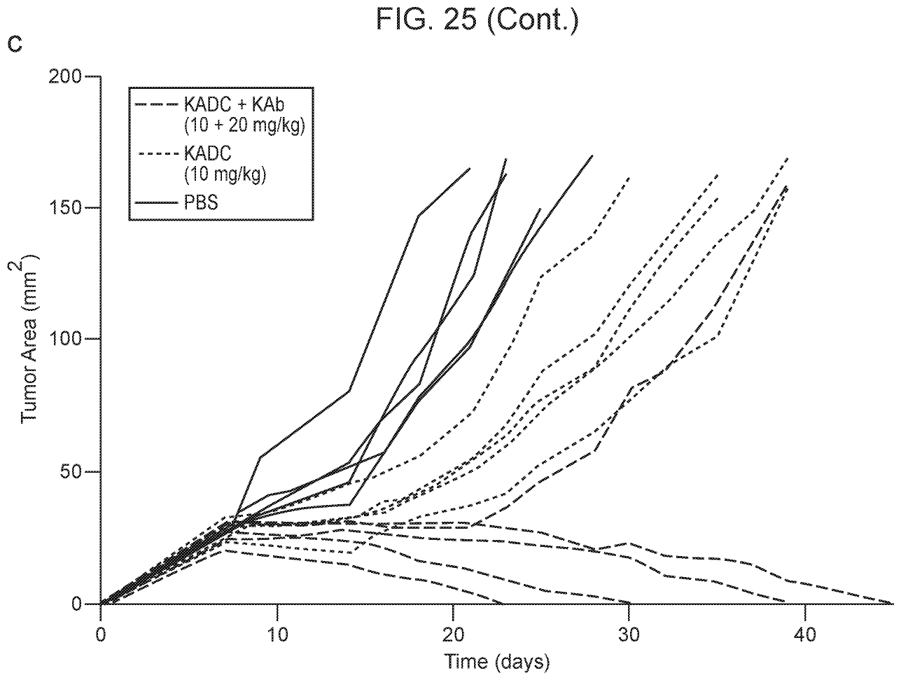

The reduction of KADC efficacy observed in the more aggressive tumor model suggests a lack of homogeneous tumor delivery as ADCs are known to suffer from limited tumor penetration in solid tumors (REF). Cilliers et al. demonstrated in 2018 that ADCs' lack of tumor penetration can be improved by coadministration with unconjugated antibody to compete for target binding sites. To test the hypothesis that heterogeneous delivery to solid tumors is an important factor explaining the limited KADC efficacy, mice were next treated with a combination of 10 mg/kg KADC and 20 mg/kg unconjugated KAb to saturate integrin receptors and promote increased tumor penetration by KADC. This combination treatment significantly increased the effectiveness of 10 mg/kg KADC, comparable to treating with a higher concentration of KADC (FIG. 25).

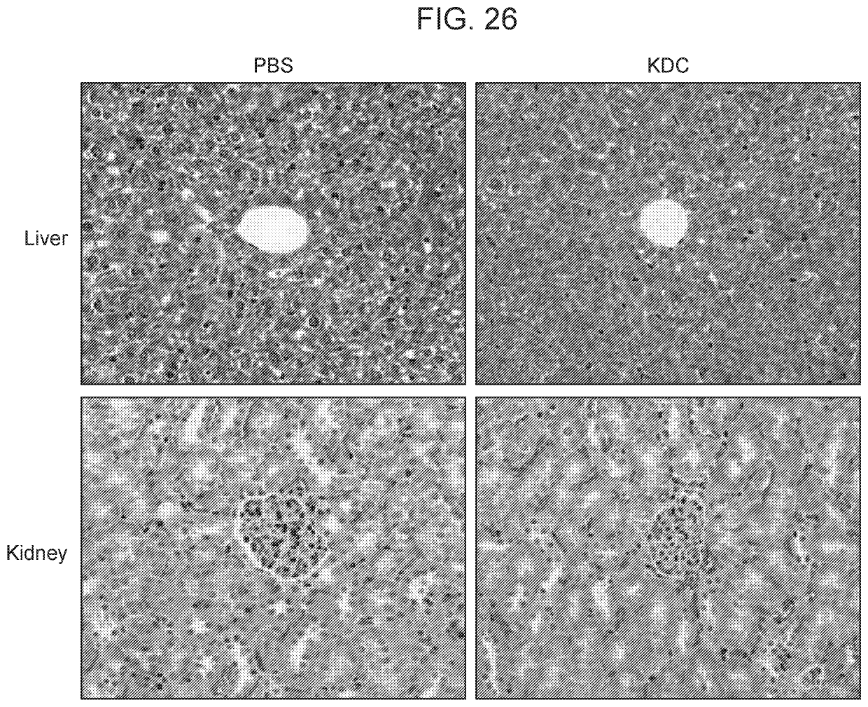

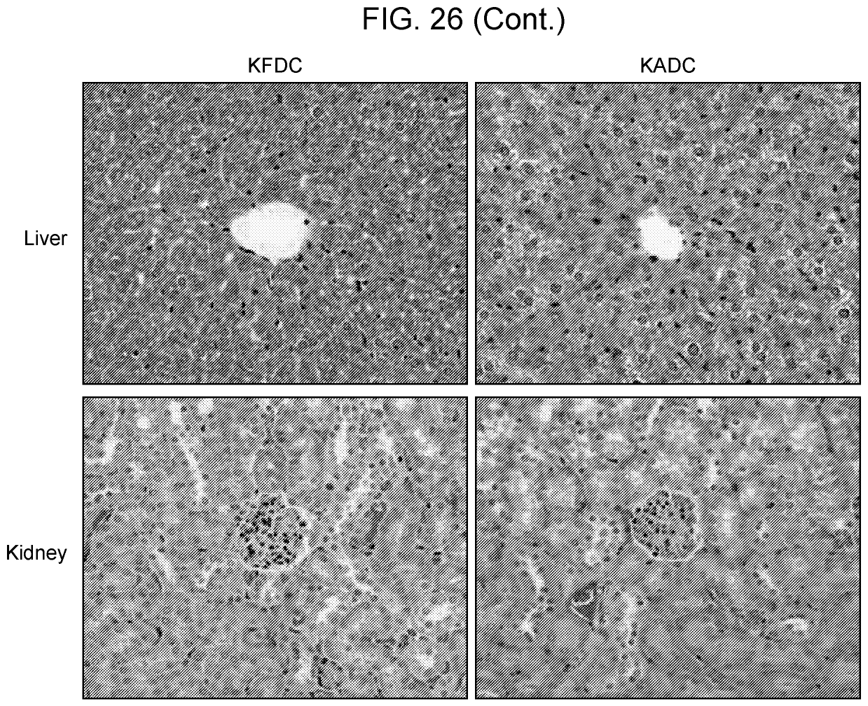

Despite the impressive in vivo efficacy of the KDCs, there are potential concerns of acute toxicity given its rapid renal filtration and high molar dose of MMAE. Although no weight loss was observed in treated mice, it was necessary to more rigorously asses organ health in treated mice. After the 60-day experiment, livers and kidneys were harvested from treated mice, fixed in 4% paraformaldehyde (PFA), then embedded in paraffin blocks for tissue sectioning. Sections were stained with hematoxylin and eosin to visualize cellular structures and scored by an independent pathologist for evidence of acute toxicity. Representative images shown in FIG. 26 were all free from indicators of toxicity and all samples were scored identically to healthy mice.

### Example 5—Mechanistic Investigations

The theoretical solid tumor uptake of intravenously administered therapeutics is predicted to be influenced by two major factors: circulatory half-life and diffusion into the tumor mass. As demonstrated in the Example 3 above, the half-life of each drug conjugate scale as expected with MW. It was therefore hypothesized that a major contributor to the differences in observed efficacy was the diffusivity of each construct.

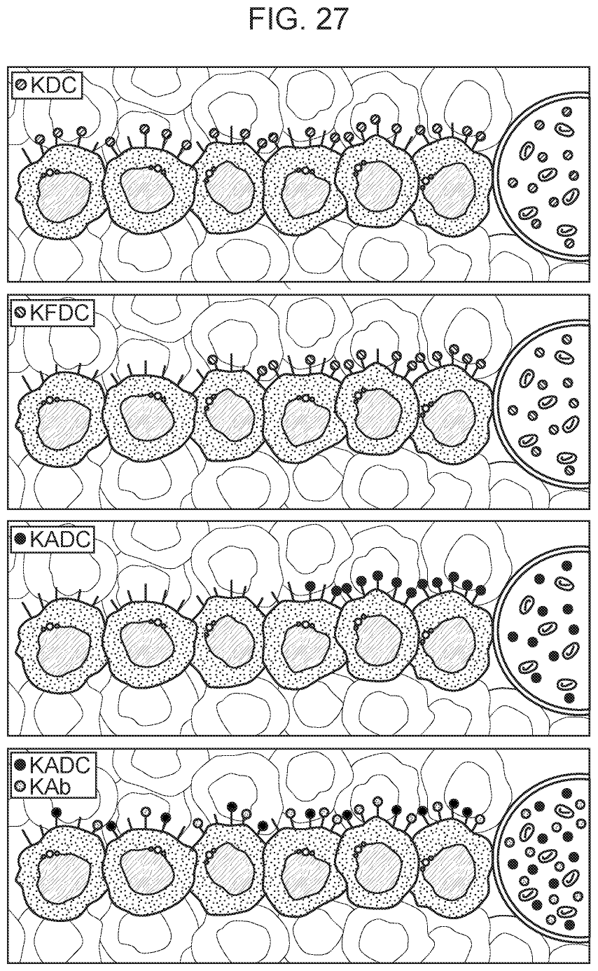

Despite high tumor uptake, antibodies and other large constructs are known to suffer from poor diffusivity and heterogeneous tumor uptake. The bulk of tumor-targeting antibodies have been shown to accumulate in the tumor periphery closest to tumor vasculature. Upon vascular extravasation, homogeneous distribution within a solid tumor is dependent upon diffusivity and rate of peripheral cellular internalization. As depicted in FIG. 27A-C, the results of the in-vivo therapy studies are explained the predicted diffusivity of each construct. Because of the KDC's rapid diffusion into solid tumors, its slower internalization rate, and its potency after only brief exposure, this construct is able to most effectively treat solid tumors. The KFDC has slower diffusion and a faster rate of internalization, consistent with its observed moderate decrease in potency in-vivo. The largest construct, KADC, has the lowest diffusivity and the fastest rate of internalization and thus performs worst under the tested conditions.

This hypothesized mechanism also explains the effect of increasing the dose of KADC to 30 mg/kg or co-administering KADC with unconjugated KAb (FIG. 27D). According to the proposed mechanism, addition of additional KADC or KAb would have a similar impact on efficacy. In each case, the increased plasma concentration results in higher perivascular concentration and effectively decreases the internalization by peripheral tumor cells is decreased by binding a higher proportion of available integrin receptors. Regardless of whether or not the additional protein is conjugated with MMAE, the increased saturation of available integrin receptors in the tumor periphery allows KADC to more homogenously diffuse away from the vasculature.

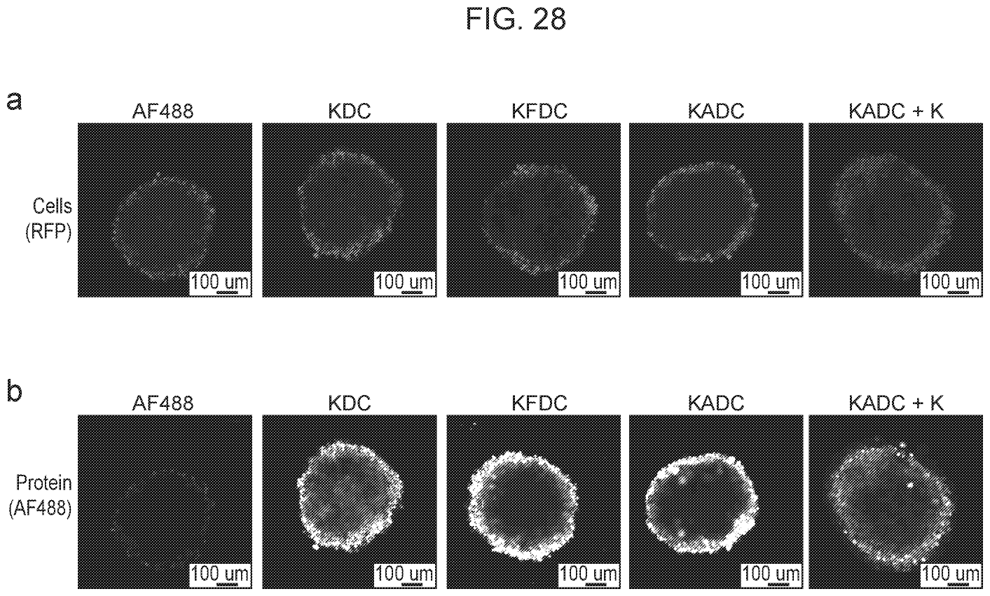

To verify this mechanism, an in-vitro setup was constructed to test and visualize differences in solid tumor diffusion. Solid tumor spheroids of RFP-expressing U87MG glioblastoma cells were grown in ultra-low adhesion 96-well plates. Spheroids were grown to until reaching a diameter of ˜750 μm then treated with 200 nM of AF488-labeled KDC, KFDC, or KADC and incubated for 4 h. Confocal microscopy was then used to image optical slices of each spheroid at a depth of 100 μm to visualize the relative intensity of labeled protein within each spheroid. As shown in FIG. 28A, the red channel control intensity is consistent among all groups while the green channel intensity is significantly higher in the center of spheroids treated with AF488-KDC. These data provide a qualitative visual confirmation that KDC is more effective at diffusing into a solid tumor mass.

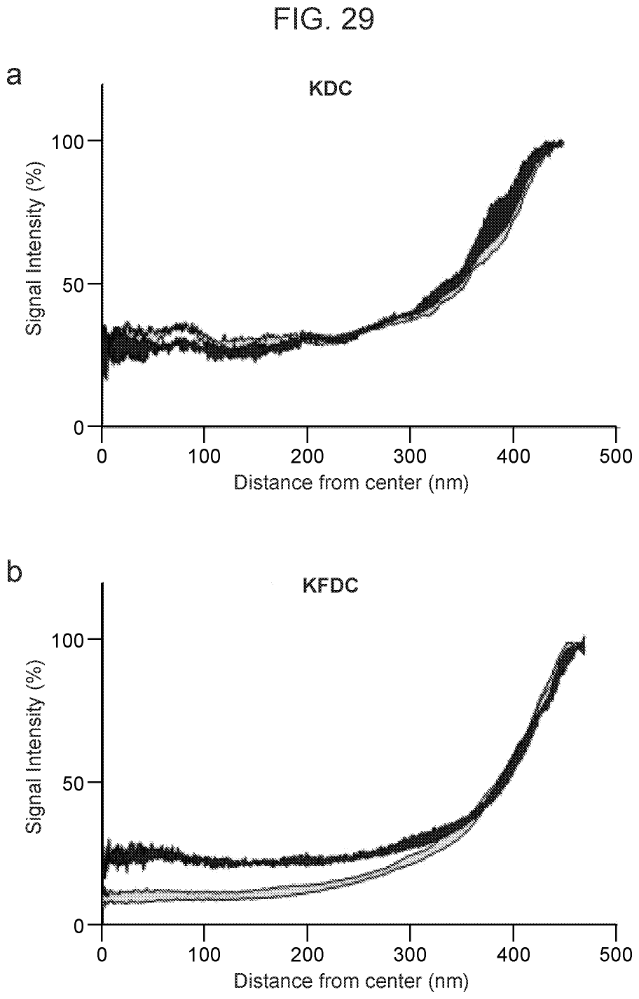

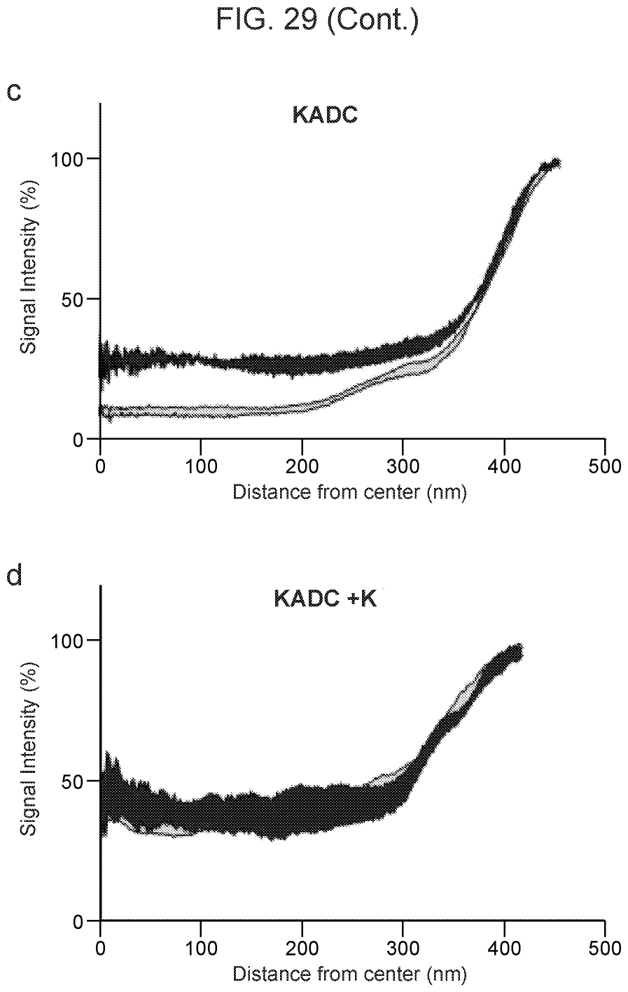

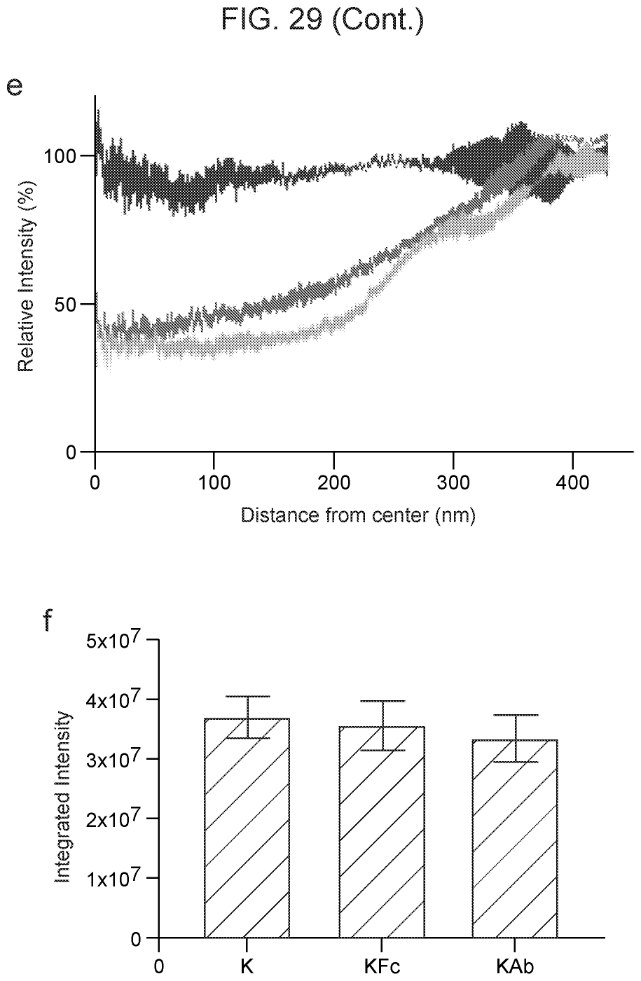

To obtain a more quantitative comparison, the relative decay of green channel intensity was compared to red channel intensity from the constitutively expressed RFP. Because the red channel should be uniform, due to stable RFP expression, and is only attenuated by the optical density of the spheroid, this signal intensity was used to correct the signal intensity of the green channel. FIG. 29 shows that using this correction, the true intensity of spheroids treated with AF488-KDC is uniform throughout the spheroid and this drug conjugate achieves homogeneous targeting while KFDC and KADC have significant signal drop-off. Moreover, the total fluorescent intensity of each spheroid was equivalent for all conjugates (FIG. 29F), further indicating that KDC homogenously diffuses into the spheroid while KFDC and KADC are more highly concentrated in periphery of the spheroid.

The mechanism was further investigated by co-administering AF488-KADC and unconjugated K or KAb to bind receptors. When incubated with equimolar K or KAb, AF488-KADC had significantly more homogeneous signal towards the center of the spheroid (FIG. 28, 29D, 29E).

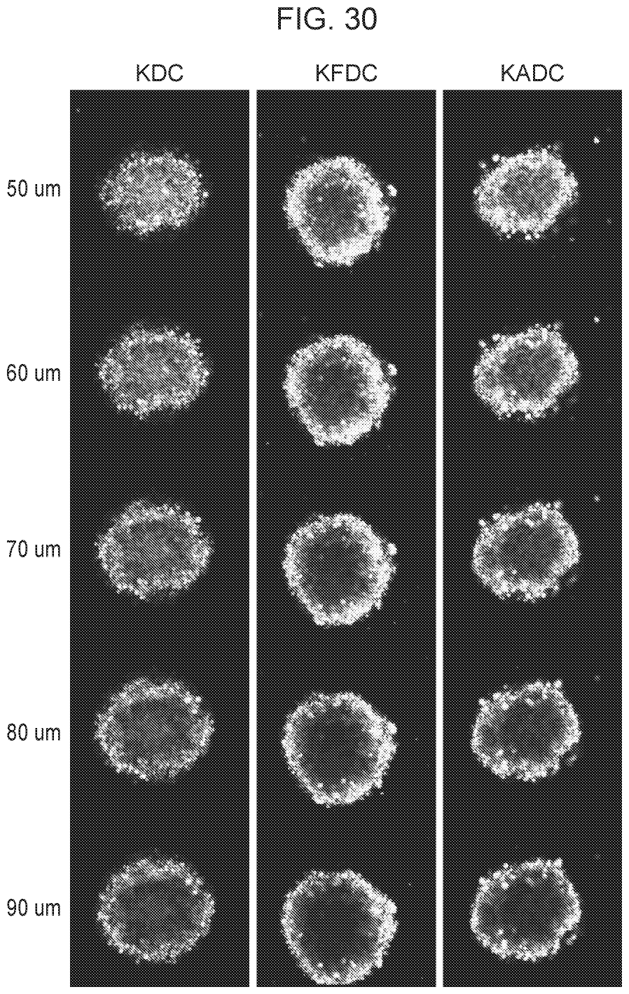

The results in FIGS. 28 and 29 were corroborated by numerous replicates over multiple independent trials. However, to further verify this finding and ensure that the result was not due to an imaging artifact, spheroids were again incubated with fluorescently-labeled proteins in the same fashion and imaged at multiple z-heights to visualize the drop-off in signal as a function of depth. As shown in FIG. 30, the green intensity in the center of each optical slice remains relatively constant in spheroids treated with KDC as compared to those treated with KFDC or KADC, where the signal drop-off is far more apparent.

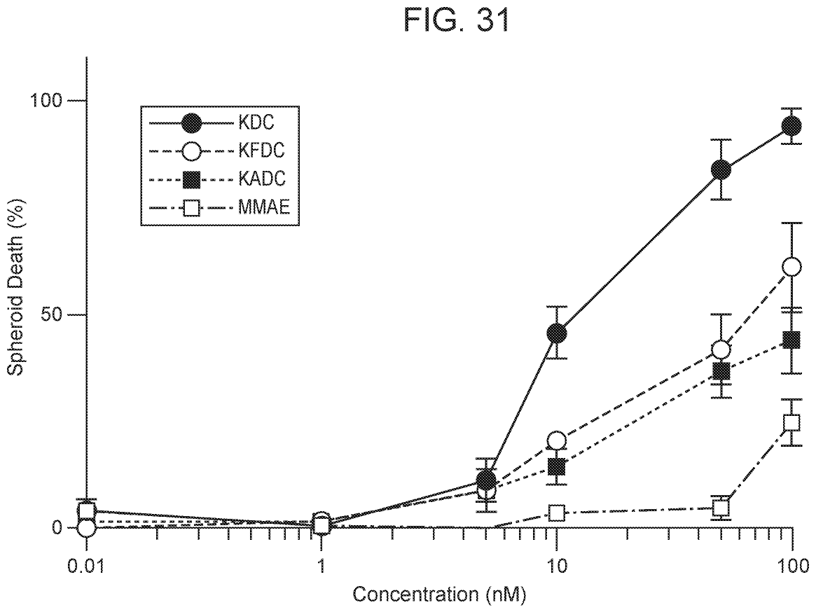

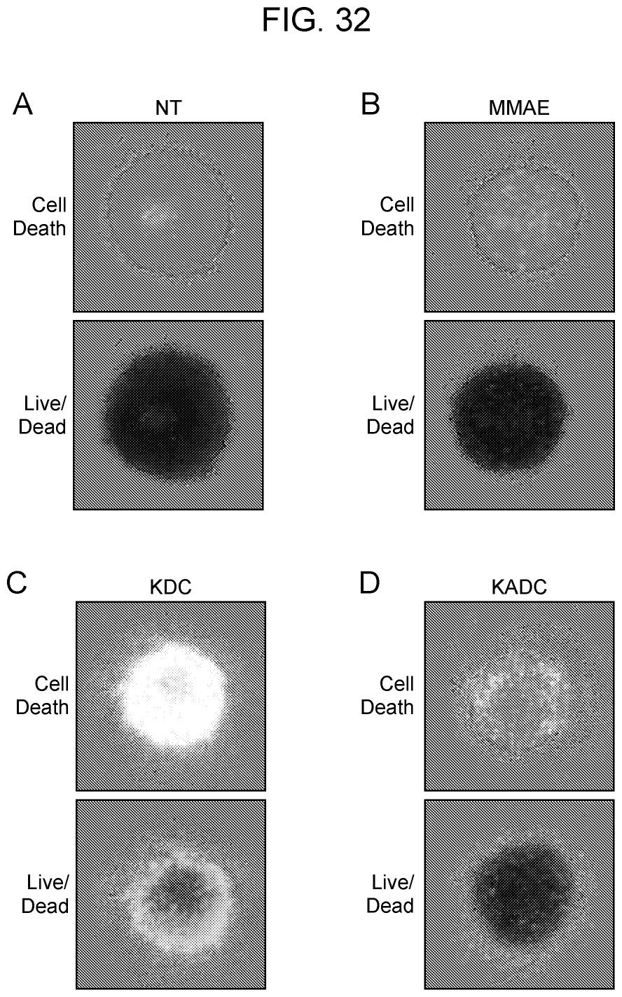

Next, this in vitro model was modified to test if similar cell killing could be visualized in addition to confocal imaging experiments. Spheroids were treated with drug conjugates or MMAE at a range of concentrations. After incubating with drug conjugates for 5 d, media was replaced with fresh media containing SYTOX green. Fluorescence measured by a BioTek Synergy H4 microtiter plate reader was compared to spheroids treated with lysis buffer. At 100 nM, KDC show substantially more killing than KFDC, KADC, or MMAE (FIG. 31). To visualize this killing over time, spheroids treated with 50 or 100 nM of drug conjugates or MMAE were imaged every 4 h using an IncuCyte. Confirming the results of the plate reader assay, KDC was highly potent and reached ˜100% spheroid toxicity while KADC had substantially decreased efficacy (FIG. 32A, 32B). Further, when co-administered with drug conjugate plus unconjugated knottin, KFDC and KADC had significantly increased potency, while KDC was equivalently effective with or without addition of unconjugated knottin (FIG. 32C, 32D).

### Materials and Methods

Synthesis of KDC (3CM-MMAE)

Solid phase peptide synthesis was used to synthesize knottin peptides on Rink amide resin using standard Fmoc conditions as described in Chapter 3. A modified version of 2.5F, termed 3CM, was synthesized with the unnatural amino acid, 5-azido-L-norvaline, in place of the serine at position 15 of 2.5F to provide a conjugation site for drug attachment. Peptide cleavage, folding, and HPLC purification were performed as previously described. The peptide contains a C-terminal amide, consistent with the use of Rink amide as the solid support.

3CM peptide (1 eq, 1.09 umol) was reacted with DBCO-Val-Cit-PAB-MMAE (1.2 eq, 1.2 umol) in 50% DMSO/50% PBS so that the final concentration of the reaction mixture with respect to 3CM would be 0.62 mM. A round bottom flask was used as the reaction vessel and was heated in a 40° C. oil bath with stirring for two days.

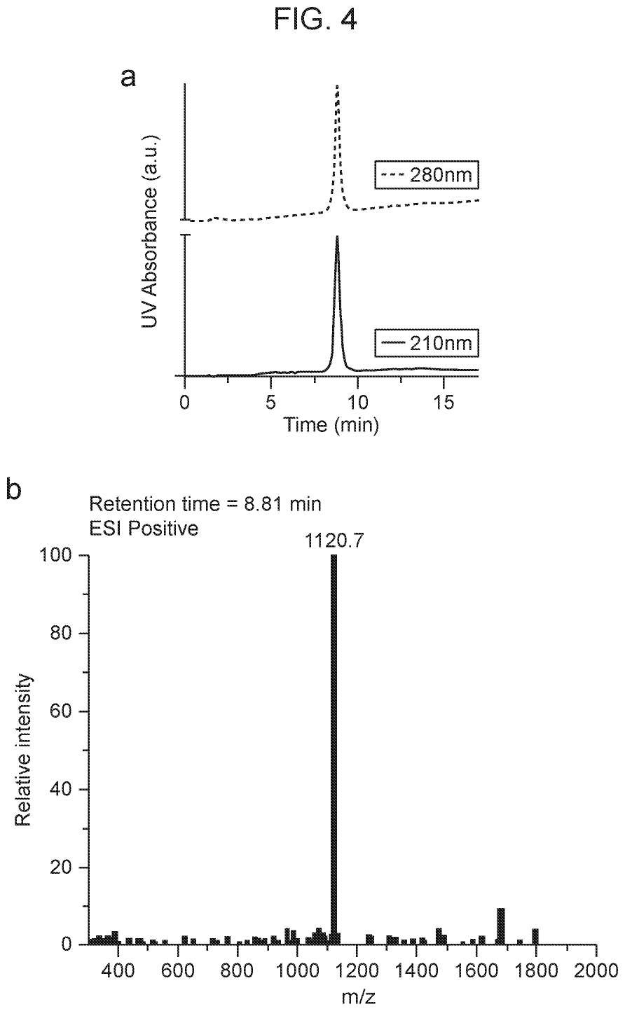

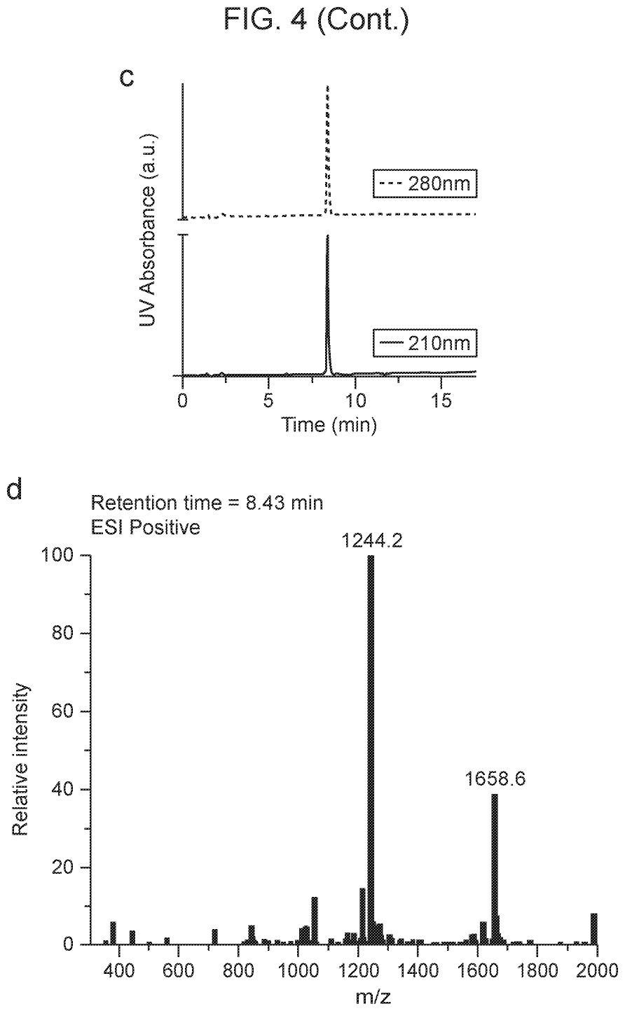

Trace solids were removed by centrifugation in a 15 mL falcon tube at 3500 xg for 5 minutes, followed by filtration through a PTFE syringe filter. At this point, the entire reaction volume was purified by RP-HPLC using a semi-prep C18 column (ZORBAX Eclipse XDB-C18, 9.4×250 mm) with a flow rate of 4 mL/min (solvent A: Milli-Q purified water+0.1% TFA; solvent B: acetonitrile+0.1% TFA). The purification method consists of a 2-minute isocratic hold at 30% solvent B, followed by a linear gradient from 30% to 100% solvent B over 30 minutes. Product elutes between 11-12 minutes. LC-MS was used to verify KDC synthesis with mass spectra of the appropriate mass confirming conjugation (3CM-MMAE UV chromatograms and mass spectra shown in FIG. 4). The LC-MS method consists of a 2-minute isocratic hold at 30% solvent B, followed by a linear gradient from 30% to 100% solvent B over 15 minutes. The knottin peptide (3CM) and knottin peptide-drug conjugate (3CM-MMAE) were characterized by low-resolution (ESI-MS) mass spectrometry based on m/z values.

Mass Spectrometry

LC-MS experiments were performed on an Agilent Technologies 1260 Infinity attached to a 6120 Quadrupole MS and a Peak Scientific NM32LA nitrogen generator. An Agilent InfinityLab Poroshell 120 EC-C18, 4.6×50 mm analytical LC column was used with a flow rate of 0.4 mL/min (solvent A: Milli-Q purified water+0.1% TFA; solvent B: acetonitrile+0.1% TFA). Wavelengths of 210 and 280 nm were monitored using the diode array detector. Data were processed and analyzed using LC/MSD ChemStation (Agilent Technologies).

Cell Lines and Cell Culture

U87MG glioblastoma cells were obtained from American Type Culture Collection (Manassas, Va.). Red Fluorescent Protein-transfected U87MG cells were obtained from AngioProteomie (Boston, Mass.). Cells were cultured in Dulbecco's Modified Eagle Medium (DMEM) supplemented with 10% Fetal Bovine Serum (FBS) and 1% penicillin-streptomycin (P/S).

Direct Binding Assays

U87MG cells were detached using Enzyme-Free Cell Dissociation Buffer (Gibco). Next, 4×10⁴ cells were incubated with varying concentrations (0.01-200 nM) of Alexa Fluor 488 labeled proteins in Integrin Binding Buffer (IBB; 25 mM Tris pH 7.4, 150 mM NaCl, 2 mM CaCl₂), 1 mM MgCl₂, 1 mM MnCl₂, and 0.1% bovine serum albumin (BSA)) for 3 h at 4° C. to minimize internalization. Cells were pelleted and washed twice with 800 μL of PBSA (phosphate buffered saline containing 0.1% bovine serum albumin) and the fluorescence of remaining surface-bound protein was measured by flow cytometry using a Guava EasyCyte 8HT instrument (EMD Millipore). Resulting data were evaluated using FlowJo software (TreeStar Inc) and equilibrium dissociation constants (Kd) were determined using Prism software (GraphPad). Error bars represent the standard deviation of experiments performed in triplicate.

Indirect Binding Assays

U87MG cells were detached using Enzyme-Free Cell Dissociation Buffer (Gibco). Next, 4×10⁴ cells were incubated with 1 nM of Alexa Fluor 488 labeled knottin as a competitor and varying concentrations (0.01-500 nM) of KDC, KFDC, or KADC in IBB for 3 h at 4° C. to minimize internalization. Cells were pelleted and washed twice with 800 μL of PBSA (phosphate buffered saline containing 0.1% bovine serum albumin) and the fluorescence of remaining surface-bound protein was measured by flow cytometry using a Guava EasyCyte 8HT instrument (EMD Millipore). Resulting data were evaluated using FlowJo software (TreeStar Inc) and equilibrium dissociation constants (Kd) were determined using Prism software (GraphPad). Error bars represent the standard deviation of experiments performed in triplicate.

Measurement of Cellular Internalization

U87MG cells were seeded in 24-well plates (Corning) at a density of 5×10⁴ cells per well. After allowing cells to settle for >4 h, media was replaced with fresh media containing 200 nM of Alexa Fluor 488-labeled proteins and supplemented with 1 mM MnCl₂. Cells were then incubated at 37° C. for 4 h. Cells were washed with 500 μL of PBS and detached using 20 μL of 0.05% trypsin/EDTA. After neutralizing trypsin and transferring each well into an Eppendorf tube, cells were washed with 800 μL of cold PBSA. Next, cells were pelleted and resuspended on ice in 50 μL of a 1:200 dilution of anti-Alexa Fluor 488 antibody (Invitrogen) to quench surface-bound protein. Cells were washed again with 800 μL of PBSA and the fluorescence of internalized protein was measured by flow cytometry as described above.

Endpoint Inhibition of Cell Proliferation

U87MG cells were seeded in a 96-well plate (Corning) at a density of 2.5×10³ cells per well and grown overnight. Cells were then treated with 100 μL of fresh media containing varying concentrations of KDC, KFDC, KADC, or MMAE and incubated for 4 d. Cell proliferation was measured using the Dokindo Cell Counting Kit-8 by replacing the media in each well with 100 μL of media containing 10% WST8. After incubation for 1 h at 37° C., absorbance (A) at 450 nm was measured with a BioTek Synergy H4 microtiter plate reader. The background signal of CCK-8 alone was subtracted from all samples. Cell proliferation was then expressed as a percentage of absorbance relative to the control of untreated cells as shown below. Nonlinear regression analysis was performed using GraphPad Prism.

$$Relative Proliferation = \frac{{A}_{treatment} - {A}_{background}}{{A}_{untreated} - {A}_{background}} \times 100 \%$$

Inhibition of Cell Proliferation and Cytotoxicity Over Time

RFP-expressing U87MG cells were seeded in a 96-well plate (Corning) at a density of 2.5×10³ cells per well and grown overnight. Cells were then treated with 200 μL of fresh media containing 10 nM Cytox Green (ThermoFisher) and KDC, KFDC, KADC, or MMAE, at the approximate ED₅₀ of 10 nM or at the saturating concentration of 100 nM. Cells were incubated in an IncuCyte S3 Live-Cell Analysis System for 5 d with images collected every 4 h. For washed conditions, media was replaced with fresh drug-containing media or fresh drug-free media after 3 h or after 24 h.

Cell Spheroid Imaging

Spheroids of RFP-expressing U87MG cells were formed by seeding round-bottom, 96-well ultra-low adhesion plates (Corning) with 4×10³ cells/well and centrifuging at 1000×g for 10 minutes. Spheroids were grown for 4 days, until reaching a diameter of 750 μm. Spheroids were then incubated in fresh media containing 200 nM of Alexa Fluor 488-conjugated KDC, KFDC, or KADC for 4 h. After incubation, spheroids were washed in PBS and transferred into a Falcon clear bottom polystyrene microplate (Corning). Spheroids were imaged using an inverted LSM 780 multiphoton laser scanning confocal microscope (Zeiss). Images shown are confocal slices taken at a depth of 100 μm from the base of the spheroid. Quantification was performed using FIJI image analysis software. Intensity was calculated radially from the center of each spheroid and plotted as the relative ratio of green intensity corrected for the intensity of RFP as an attenuation control.

Endpoint Spheroid Killing Assays

Spheroids of U87MG cells were formed by seeding round-bottom, 96-well ultra-low adhesion plates (Corning) with 4×10³ cells/well and centrifuging at 1000×g for 10 minutes. Spheroids were grown for 2 days before replacing media with 200 μL of fresh media containing 10 nM Cytox Green (ThermoFisher) and a range of concentrations of KDC, KFDC, KADC, or MMAE. After 4 d, trypsin-EDTA was added and cells were placed on a 60 RPM shaker for 10 min to disrupt spheroids. Green fluorescence was measured using was measured with a BioTek Synergy H4 microtiter plate reader. Percent toxicity was calculated by comparing green fluorescence to spheroids treated with lysis buffer.

Cell Spheroid Killing Assays Over Time

Spheroids of RFP-expressing U87MG cells were formed by seeding round-bottom, 96-well ultra-low adhesion plates (Corning) with 4×10³ cells/well and centrifuging at 1000×g for 10 minutes. Spheroids were grown for 2 days before replacing media with 200 μL of fresh media containing 10 nM Cytox Green (ThermoFisher) and 0, 50, or 100 nM KDC, KFDC, KADC, or MMAE. Spheroids were incubated in an IncuCyte S3 Live-Cell Analysis System for 5 d with images collected every 4 h.

Animal Experiments

For tumor cell implantation, female Nu/Nu mice (Charles River Laboratory) were anesthetized with 2.5% isoflurane by inhalation with a flow rate of 1 L/min. A volume of 200 μL of 50/50 PBS/Matrigel (Corning), containing 2.5×10⁶ U87MG cells, was injected subcutaneously into the right flank. Tumors were allowed to grow for 7 days until reaching a size of approximately 35 mm². For therapeutic efficacy studies, mice were binned into experimental groups to ensure equivalent average tumor sizes and starting body weights across all groups.

In Vivo Time Course Imaging

Mice bearing U87MG flank xenografts, as described above, were injected intravenously with 1.5 nmol of Alexa Fluor 680-labeled K, KFc, or KAb. Mice were imaged using an IVIS Spectrum (PerkinElmer). The fluorophore-conjugated protein was detected using excitation/emission wavelengths of 640 nm and 710 nm, respectively. All imaging analysis was performed using Living Image software (Caliper Life Sciences).

Ex Vivo Biodistribution

Mice bearing U87MG flank xenografts, as described above, were injected intravenously with 1.5 nmol of Alexa Fluor 680-labeled K, KFc, or KAb. Organs (tumor, liver, kidneys, spleen, pancreas, heart, lungs, bladder, quadriceps muscle, and bone) were collected at end points of 4 h and 24 h. Organs were imaged using an IVIS Spectrum (PerkinElmer). The fluorophore-conjugated protein was detected using excitation/emission wavelengths of 640 nm and 710 nm, respectively. All imaging analysis was performed using Living Image software (Caliper Life Sciences).

Visualization of Extravasation Depth into Tumor

Mice bearing U87MG flank xenografts, as described above, were injected intravenously with 1.5 nmol of Alexa Fluor 480-labeled K, KFc, or KAb. Tumors were removed after 4 h and snap frozen in OCT Optimal Cutting Temperature compound (OCT; ThermoFisher Scientific). 15 minutes before euthanization, mice were also injected intravenously with 15 mg/kg Hoechst 33342 to visualize functional vasculature. OCT blocks were cut into 15 μm slices and slides were stained using Cy5.5-labeled anti-CD31. Stained slides were then imaged using an inverted LSM 780 multiphoton laser scanning confocal microscope (Zeiss).

Therapeutic Efficacy Experiments

Mice bearing U87MG flank xenografts, as described above, were injected intravenously with PBS, KDC, KFDC, KADC, or controls proteins. Treatments were administered once per week for four weeks. Tumors were measured three times per week using digital calipers and animal weights were recorded on each dosing day to monitor for potential weight loss as a measure of compound toxicity. Tumor area was calculated as the longest axis of the tumor multiplied by the perpendicular axis. Euthanasia criteria were defined as a tumor area greater than 150 mm² or a loss of 20% body weight.

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

What is claimed is:

1. A conjugate, comprising:
a knottin peptide comprising an engineered loop that binds to a cell surface molecule; and
an anti-microtubule agent conjugated to the knottin peptide via a linker.

2. The conjugate of claim 1, wherein the anti-microtubule agent is a tubulin inhibitor.

3. The conjugate of claim 2, wherein the tubulin inhibitor is an auristatin.

4. The conjugate of claim 3, wherein the tubulin inhibitor is an auristatin E or auristatin F.

5. The conjugate of claim 4, wherein the tubulin inhibitor is monomethylauristatin E (MMAE) or monomethylauristatin F (MMAF).

6. The conjugate of any one of claims 1 to 5, wherein the linker is an enzyme cleavable linker.

7. The conjugate of claim 6, wherein the enzyme cleavable linker is a protease cleavable linker.

8. The conjugate of claim 7, wherein the linker comprises a valine-citrulline dipeptide, a valine-alanine dipeptide, or both.

9. The conjugate of claim 8, wherein the linker is a valine-citruline-paraaminobenzyloxy (Val-Cit-PAB) linker.

10. The conjugate of claim 8, wherein the linker is a valylalanylparaaminobenzyloxy (Val-Ala-PAB) linker.

11. A conjugate, comprising:
a fusion protein, comprising:
a knottin peptide comprising an engineered loop that binds to a cell surface molecule, fused to
an antibody subunit or fragment thereof; and
a drug conjugated to the fusion protein via a linker.

12. The conjugate of claim 11, wherein the antibody subunit or fragment thereof is an antibody heavy chain or fragment thereof.

13. The conjugate of claim 12, wherein the antibody heavy chain or fragment thereof comprises a γ, α, δ, ε, or μ antibody heavy chain or fragment thereof.

14. The conjugate of claim 13, wherein the antibody heavy chain or fragment thereof is an IgG heavy chain or fragment thereof.

15. The conjugate of claim 14, wherein the IgG heavy chain or fragment thereof is an IgG1 heavy chain or fragment thereof.

16. The conjugate of any one of claims 12 to 15, wherein the antibody heavy chain or fragment thereof comprises a heavy chain variable region (V_{H}).

17. The conjugate of claim 16, wherein the antibody heavy chain or fragment thereof further comprises a heavy chain constant region or fragment thereof.

18. The conjugate of claim 17, wherein the antibody heavy chain constant region or fragment thereof comprises a C_{H}1 domain.

19. The conjugate of claim 18, wherein the antibody heavy chain constant region or fragment thereof further comprises a C_{H}2 domain.

20. The conjugate of claim 19, wherein the antibody heavy chain constant region or fragment thereof further comprises a C_{H}3 domain.

21. The conjugate of claim 20, wherein the antibody heavy chain or fragment thereof is a full-length antibody heavy chain.

22. The conjugate of any one of claims 12 to 15, wherein the antibody heavy chain or fragment thereof does not comprise a V_{H}.

23. The conjugate of claim 22, wherein the antibody heavy chain or fragment thereof comprises an Fc region.

24. The conjugate of any one of claims 12 to 23, wherein the knottin peptide is fused to the N-terminus of the antibody heavy chain or fragment thereof.

25. The conjugate of any one of claims 12 to 23, wherein the knottin peptide is fused to the C-terminus of the antibody heavy chain or fragment thereof.

26. The conjugate of any one of claims 12 to 25, wherein the drug is conjugated to the knottin peptide.

27. The conjugate of any one of claims 12 to 25, wherein the drug is conjugated to the antibody heavy chain or fragment thereof.

28. The conjugate of claim 27, wherein the antibody heavy chain or fragment thereof comprises an Fc region, and wherein the drug is conjugated to the Fc region.

29. The conjugate of claim 28, wherein the drug is conjugated to the C_{H}3 domain of the Fc region.

30. The conjugate of claim 28, wherein the drug is conjugated to the C_{H}2 domain of the Fc region.

31. The conjugate of claim 11, wherein the antibody subunit or fragment thereof is an antibody light chain or fragment thereof.

32. The conjugate of claim 31, wherein the antibody light chain or fragment thereof is a kappa (κ) light chain or fragment thereof.

33. The conjugate of claim 31, wherein the antibody light chain or fragment thereof is a lambda (λ) light chain or fragment thereof.

34. The conjugate of any one of claims 31 to 33, wherein the antibody light chain or fragment thereof comprises an antibody light chain variable region (V_{L}).

35. The conjugate of claim 34, wherein the antibody light chain or fragment thereof further comprises an antibody light chain constant region (C_{L}) or fragment thereof.

36. The conjugate of any one of claims 31 to 35, wherein the antibody light chain or fragment thereof is a full-length antibody light chain.

37. The conjugate of any one of claims 31 to 36, wherein the knottin peptide is fused to the N-terminus of the antibody light chain or fragment thereof.

38. The conjugate of any one of claims 31 to 36, wherein the knottin peptide is fused to the C-terminus of the antibody light chain or fragment thereof.

39. The conjugate of any one of claims 31 to 38, wherein the drug is conjugated to the knottin peptide.

40. The conjugate of any one of claims 31 to 38, wherein the drug is conjugated to the antibody light chain or fragment thereof.

41. The conjugate of any one of claims 11 to 40, wherein the drug is a cytotoxic agent.

42. The conjugate of any one of claims 11 to 40, wherein the drug is a toxin.

43. The conjugate of any one of claims 11 to 40, wherein the drug is an anti-microtubule agent.

44. The conjugate of claim 43, wherein the anti-microtubule agent is a tubulin inhibitor.

45. The conjugate of claim 44, wherein the tubulin inhibitor is an auristatin.

46. The conjugate of claim 45, wherein the tubulin inhibitor is an auristatin E or auristatin F.

47. The conjugate of claim 46, wherein the tubulin inhibitor is monomethylauristatin E (MMAE) or monomethylauristatin F (MMAF).

48. The conjugate of any one of claims 11 to 40, wherein the drug is a nucleoside drug.

49. The conjugate of claim 48, wherein the nucleoside drug is a nucleoside analogue.

50. The conjugate of claim 49, wherein the nucleoside analogue is selected from the group consisting of: gemcitabine, cytarabine, troxacitabine, decitabine, cladribine, fludarabine, clofarabine, and 2′-C-cyano-2′-deoxy-1-β-D-arabino-pentofuranosylcytosine (CNDAC).

51. The conjugate of any one of claims 11 to 50, wherein the linker is an enzyme cleavable linker.

52. The conjugate of claim 51, wherein the enzyme cleavable linker is a protease cleavable linker.

53. The conjugate of claim 52, wherein the linker comprises a valine-citrulline dipeptide, a valine-alanine dipeptide, or both.

54. The conjugate of claim 53, wherein the linker is a valine-citruline-paraaminobenzyloxy (Val-Cit-PAB) linker.

55. The conjugate of claim 53, wherein the linker is a valylalanylparaaminobenzyloxy (Val-Ala-PAB) linker.

56. The conjugate of any one of claims 1 to 55, wherein the knottin peptide is selected from the group consisting of: an EETI-II peptide, an AgRP peptide, a ω-conotoxin peptide, a Kalata B1 peptide, an MCoTI-II peptide, an agatoxin peptide, and a chlorotoxin peptide.

57. The conjugate of any one of claims 1 to 56, wherein the cell surface molecule is a cancer cell surface molecule.

58. The conjugate of claim 57, wherein the cancer cell surface molecule is present on cancer cells of a solid tumor.

59. The conjugate of any one of claims 1 to 57, wherein the cell surface molecule is a cell surface receptor.

60. The conjugate of claim 59, wherein the cell surface receptor is a cell adhesion receptor.

61. The conjugate of claim 60, wherein the cell adhesion receptor is an integrin.

62. The conjugate of claim 60, wherein the integrin is selected from the group consisting of: αvβ1 integrin, αvβ3 integrin, αvβ5 integrin, αvβ6 integrin, α5β1 integrin, and any combination thereof.

63. The conjugate of claim 59, wherein the cell surface receptor is a chemokine receptor.

64. The conjugate of claim 63, wherein the chemokine receptor is C—X—C chemokine receptor type 4 (CXCR4).

65. The conjugate of claim 59, wherein the cell surface receptor is a growth factor receptor.

66. The conjugate of claim 59, wherein the cell surface receptor is an immune cell receptor.

67. The conjugate of claim 66, wherein the immune cell receptor is cytotoxic T-lymphocyte-associated protein 4 (CTLA-4).

68. The conjugate of claim 59, wherein the cell surface receptor is neuropilin-1 (NRP1).

69. The conjugate of any one of claims 1 to 57, wherein the cell surface molecule is a membrane protease.

70. The conjugate of claim 69, wherein the membrane protease is matriptase.

71. A composition comprising the conjugate of any one of claims 1 to 70.

72. The composition of claim 71, wherein the composition is a pharmaceutical composition comprising:
the conjugate; and
a pharmaceutically acceptable carrier.

73. The composition of claim 72, wherein the pharmaceutical composition is formulated for parenteral administration.

74. The composition of claim 72, wherein the pharmaceutical composition is formulated for oral administration.

75. A kit comprising:
a therapeutically effective amount of the pharmaceutical composition of claim 72; and
instructions for administering the pharmaceutical composition to an individual in need thereof.

76. The kit of claim 75, wherein the pharmaceutical composition is present in one or more unit dosages.

77. A method comprising administering a therapeutically effective amount of the pharmaceutical composition of claim 72 to an individual in need thereof.

78. The method according to claim 77, wherein the individual has cancer and the engineered loop binds to a cell surface molecule on cancer cells present in the individual.

79. The method according to claim 78, wherein the cell surface molecule is a cell surface receptor.

80. The method according to claim 79, wherein the cell surface receptor is a cell adhesion receptor.

81. The method according to claim 80, wherein the cell adhesion receptor is an integrin.

82. The method according to claim 81, wherein the integrin is selected from the group consisting of: αvβ1 integrin, αvβ3 integrin, αvβ5 integrin, αvβ6 integrin, α5β1 integrin, and any combination thereof.

83. The method according to claim 82, wherein the individual has a solid tumor comprising the cancer cells.

84. The method according to claim 83, wherein the solid tumor is a brain tumor.
